(19) United States
(12) Patent Application Publication
(10) Pub. No.: US 2012/0045419 A1
(43) Pub. Date: Feb. 23, 2012

(54) METHODS AND USES OF HYPOXIC COMPARTMENT CELLS

(75) Inventors: William Mark Erwin, North York (CA); Michael George Fehlings, Toronto (CA); Robert Davies Inman, Toronto (CA)

(73) Assignee: UNIVERSITY HEALTH NETWORK, Toronto, ON (CA)

(21) Appl. No.: 13/148,432

(22) PCT Filed: Feb. 5, 2010
(86) PCT No.: PCT/CA2010/000169

§ 371 Date: Nov. 14, 2011

Related U.S. Application Data
(60) Provisional application No. 61/150,565, filed on Feb. 6, 2009.

(51) Int. Cl. A61K 35/12; C12N 5/0797; A61P 25/00; C12P 1/00; A61K 38/18; C12N 5/071; C12Q 1/02

(52) U.S. Cl. 424/93.7; 435/325; 435/373; 435/375; 435/29; 435/41; 424/115; 514/7.6; 435/377

(57) ABSTRACT

The disclosure relates to methods of maintaining and/or expanding an in vitro population of hypoxia compartment cells comprising culturing said population of cells optionally in an oxygen controlled environment, wherein the population of hypoxia compartment cells is exposed to an oxygen concentration of between about 1.5% and about 10%, preferably between about 2% and about 5%, and uses of cells expanded according to these methods.

### RELATED APPLICATIONS

This application claims the benefit of 35 USC 119 based on the priority of co-pending U.S. Provisional Patent Application U.S. 61/150,565, filed Feb. 6, 2009, which is being incorporated herein in its entirety by reference.

### FIELD

The disclosure relates to methods of maintaining and expanding hypoxic compartment cells and uses thereof and specifically to methods of maintaining and expanding nucleus pulposus cells and uses thereof.

### BACKGROUND

Degenerative disc disease (DDD) is an extremely common and expensive healthcare ailment which unlike advances in biologic therapies for fracture management and disease modifying drugs for various arthropathies has no curative strategy which attenuates or reverses the degenerative cascade [1].

It has previously been shown that non-chondrodystrophic canine intervertebral discs-derived notochordal cells secrete Connective Tissue Growth Factor (CTGF/CCN-2) and that conditioned medium obtained from these cells up-regulates important matrix gene expression, cell proliferation and proteoglycan production in nucleus pulposus (NP) cells [4, 5].

Nucleus pulposus cells (NPC) have necessarily adapted to survive within a hypoxic and relatively nutrient-poor environment, the oxygen concentration within this compartment reportedly being between 2-5% [9]. However most studies concerning intervertebral disc metabolism within the in vitro setting have studied these cells cultured under classical tissue culture conditions of 21% O_{2 [}3-5, 12-16]. A number of reports concerning the delivery of some of these factors have claimed effective restoration of disc height in vivo and/or increased expression of desirable genes such as aggrecan and collagen. However the tissue culture periods used are often only a matter of days and the cultures are also often maintained within monolayer. Further, nucleus pulposus cells assume a fibroblastic phenotype in monolayer culture which is very different from that found in the in vivo setting [16-18].

### SUMMARY

An aspect includes a method of maintaining and/or expanding an in vitro population of hypoxia compartment cells comprising culturing said population of cells under suitable cell culture conditions, wherein the population of hypoxia compartment cells is exposed to an oxygen concentration of between about 1.5% and about 10%, preferably between about 2% and about 5%, optionally in an oxygen controlled environment.

In an embodiment, the oxygen concentration selected allows the population of cells to produce cell aggregates comprising one or more of extracellular matrix, proteoglycan, and/or fibrils, wherein the cell aggregates are optionally oval or crescent shaped, optionally with viscoelastic properties.

In an embodiment, the oxygen concentration is about 3.5%

Another aspect includes a method of generating and/or enriching for progenitor cells comprising: providing a population of cells comprising and/or consisting of hypoxia compartment cells; culturing the population of cells under suitable conditions, wherein the population cells is exposed to an oxygen concentration of between about 1.5% and about 10%, preferably between about 2% and about 5%, optionally in an oxygen controlled environment for a sufficient time for the population of hypoxia compartment progenitor cells to form spherelike conformations; and isolating the spherelike conformations comprising progenitor cells.

In an embodiment, the progenitor cells express Beta III tubulin, Nestin, GFAP, NCAM, Sox2 and/or Oct3/4.

A further aspect includes a method for testing sensitivity of a population of hypoxia compartment cells to a test agent or combination, comprising maintaining and/or expanding an in vitro population of hypoxia compartment cells according to a method described herein, adding the test agent or combination and determining sensitivity of said cells to test agent or combination.

Yet a further aspect includes a method of producing a therapeutic hypoxia compartment cell comprising isolating a hypoxia compartment cell and maintaining and/or expanding said cells according to a method described herein, and optionally isolating the cell.

Another aspect includes an isolated cell, isolated or derived from a hypoxia compartment tissue, maintained and/or expanded according to a method described herein.

A further aspect includes a method of producing nucleus pulposus conditioned medium (NPCM) or notochordal cell conditioned medium (NCCM) comprising culturing a population of NP cells and/or notochordal cells, preferably primary notochordal cells, in a cell culture medium under suitable cell culture conditions, wherein the population of NP cells and/or notochordal cells is exposed to an oxygen concentration of between about 1.5% and about 10%, preferably between about 2% and about 5%, and cultured preferably in basal culture medium devoid of serum and/or growth factors, for a suitable culture period, for example of at least 12 hours, at least 24 hours, at least 36 hours, at least 48 hours, at least 60 hours or at least 72 hours, optionally in an oxygen controlled environment, thereby producing NPCM and/or NCCM.

Another aspect includes a method of inhibiting nucleus pulposus cell death comprising contacting a nucleus pulposus cell that is being exposed or will be exposed to a cell death signal with the NP or NC conditioned medium described herein, and/or contacting the nucleus pulposus cell with CTGF, preferably recombinant CTGF, more preferably recombinant human CTGF (rhCTGF) and/or an isolated cell or population described herein, and/or a cell that expresses CTGF.

Another aspect includes a method of inhibiting nucleus pulposus degeneration in a subject comprising administering, or contacting a population of the subject's NP cells, with a nucleus pulposus cell conditioned medium (NPCM) or a notochordal cell conditioned medium (NCCM) described herein, CTGF or an isolated cell or population described herein.

Yet a further aspect includes a method of treating degenerative disc disease (DDD) in a subject comprising administering NPCM or NCCM according to any one of claims 31 to 36, CTGF or an isolated cell or population of any one of claims 28 to 30, to a subject in need thereof.

A further aspect includes a method of treating a neuronal disease characterized by loss of cells, for example spinal cord injury, comprising administering NPCM or NCCM described herein, CTGF or an isolated cell or population described herein.

Also provided in another aspect is a method of differentiating a cell comprising obtaining a spherelike conformation for example according to any one of the methods described herein, culturing the spherelike conformation in a differentiation culture medium under suitable conditions to obtain a differentiated cell.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be described in relation to the drawings in which:

FIG. 1: (A) Notochordal cell construct. Cells were cast as cell suspensions and cultured at 3.5% oxygen for 4 weeks (Advanced DMEM/F-12 supplemented with PSF and 8% FCS). The construct initially appeared as a crescent shaped structure that spontaneously developed an oval shape and is depicted by black arrowhead. The construct demonstrated viscoelastic properties and hysteresis by resistance to compression by forceps and return to initial shape after compression. (B) Safranin-O staining of tissue construct. Arrowheads depict areas of intense pinkish staining around cells and within the extracellular matrix indicating robust proteoglycan content. (C) Scanning electron microscope images of notochordal cells grown from cell suspensions within monolayer culture 21% oxygen (normoxia), (D) 3.5% oxygen (hypoxia). The hypoxic cultures clearly depict the development of complex 3-D fibrils where the normoxic cultures have only a uniform monolayer.

FIG. 2: (A-B) Notochordal cells cultured under hypoxic or normoxic conditions:

(A) Marked clusters of cell growth within and at alginate globule periphery under hypoxia indicated by black arrowheads. (B) Normoxic culture demonstrates no cell growth or “cluster” organization within alginate globule-cells appear as only single and granular in appearance (black arrowheads) (×10)

(C-D) Toluidine Blue stained sections of notochordal globules:

(C) Notochordal globules cultured in hypoxia. Note intense metachromatic and nuclear staining and well defined cell membranes, clear cytoplasm in many cells and matrix within others and clearly defined cell nucleus (×40) (scale bar=100 μm). (D) Identical sourced cells cultured using 21% O₂ demonstrating cells that do not appear viable, poorly organized extracellular matrix and lack of cellular detail (×40) (scale bar=100 μm).

(E-F) Safranin-O stained sections of notochordal globules.

(E) Notochordal cells cultured in hypoxia. Note clearly defined cell membranes, nucleus and intense Safranin-O staining (×40) (scale bar=100 μm). (F) Notochordal cells cultured in normoxia showing a lack of well defined nuclei that are small, lack a clearly defined cell membrane, fibrillary appearing matrix and ill defined nuclei (×40) (scale bar=100 μm).

(G) Close-up of notochordal cell-rich nucleus pulposus (in vivo) taken from snap-frozen samples directly from animal and stained with Safranin-O (×40) (scale bar=100 μm).

FIG. 3: (A) Freshly obtained non-chondrodystrophic canine intervertebral disc pulposus fixed in 4% paraformaldehyde, paraffin embedded and stained for anti-collagen type II (black arrow). There is diffuse immune-like reactivity to anti-collagen type II staining (brown) throughout the physilliferous appearing nucleus (×40) (scale bar=100 μm). (B) Construct cultured 5 months under hypoxic conditions within alginate globules demonstrating robust immune-reactivity for anti-collagen type II (black arrows) (×40) (scale bar=100 μm). (C) Parallel normoxic cultured sample stained for collagen type II depicting only cellular debris and no visible staining for collagen type II (×40) (scale bar=100 μm). (D) Freshly obtained non-chondrodystrophic canine nucleus pulposus fixed in 4% paraformaldehyde, paraffin embedded and stained for anti-aggrecan demonstrating robust immune-like reactivity to aggrecan (black arrows) (×40) (scale bar=100 μm). (E) Construct cultured 5 months within alginate globules demonstrating strong immune-reactivity for anti-aggrecan (black arrows) (×20) (scale bar=100 μm). (F) Normoxic cultured notochordal cells demonstrating sparse immune-like reactivity to anti-aggrecan, no defined cell membranes, little evidence of extracellular accumulation or cell clusters (×20) (scale bar=100 μm).

FIG. 4: Safranin-O stained sections of (A) normoxic and (B) hypoxic cultured notochordal cells within alginate ‘globules’. The hypoxic cultured cells are larger, have clearly apparent cell membrane, large nuclei and intense Safranin-O staining. (B) Normoxic cells lack a cell membrane, have an indistinct nucleus and poor Safranin-O staining that appears fragmented and poorly organized. (C) Box-plot depiction of cell volume determined using histomorphometric methods. The mean area of notochordal cells cultured under hypoxic conditions is 328.7 units whereas it is 111.34 for cells cultured under normoxia. These differences are statistically significant at p>0.0001 and reflect 200 separate cell counts from representative sections of Safranin-O stained specimens (Mann-Whitney U test).

FIG. 5: (A) Scanning electron microscopy images of tissue construct formed within alginate globule depicting tissue construct with tightly connected cells within a complex 3-D arrangement (black arrows) (×1000). (B) Identical cells cultured under normoxia depicting only the denatured alginate ‘skeleton’ that remains (dotted arrow). There are no remaining complex cellular or extracellular matrix structures (×900) (dotted black arrowheads). (C) Stiffening adhesions between notochordal cells within a large, complex cellular construct grown for 5 months under hypoxic conditions (black arrow) (×5000).

FIG. 6: Flow Cytometry (FACS) analysis of bovine NP cells treated with (A) 0% FBS control, (B) IL-1β+FasL or (C) IL-1β+FasL+NCCM and labeled with Propidium Iodide (PI) (Y axis) and Annexin-V (X axis). (B) Demonstrates an increase in the PI and Annexin-V positive cell fraction (upper right quadrant=dead cells) as compared to either (A) or (C). PI positive and Annexin-V negative=necrosis; PI positive and Annexin-V positive=apoptotic cell death; total cell death is reflected by PI staining. Treatment with NCCM results in far fewer cells that are PI/Annexin-V positive with an appearance that is almost equivalent to baseline (A). The data are quantified in FIG. 7 below.

FIG. 7: Comparison of cell death induced by IL-1β+FasL and IL-1β+FasL+NCCM. Basal medium is Advanced DMEM/F-12 without FBS supplementation (0%). The differences between IL-1β+FasL and IL-1β+FasL+NCCM in terms of both total and apoptotic cell death are statistically significant at p=0.016 and p=0.0002 respectively. Treatment with IL-1β+FasL+NCCM results in most dead cell death as a function of apoptotic, not necrotic cell death.

FIG. 8: Real-time RT-PCR(RT-PCR) relative gene expression for (A) collagen type II, (B) Aggrecan, (C) Link Protein and (D) the CD44 receptor expressed by bovine NP cells. These data for each gene represented is expressed relative to the basal (untreated) conditions. The expression of the relevant genes was normalized to the constitutively expressed gene HPRT and the data expressed using the ^{ΔΔ}CT method. All RT-PCR experiments were repeated in quadruplicate and are presented above with standard error calculations performed for all conditions.

FIG. 9: Real-time RT-PCR (RT-PCR) relative gene expression for (A) MMP-3, (B) ADAMTS-4 and (C) TIMP-1 (matrix remodeling genes) expressed by bovine NP cells. As in FIG. 8 each gene represented is expressed relative to basal (untreated) conditions and normalized to the constitutively expressed gene HPRT. The ^{ΔΔ}CT relative gene expression method was used. All RT-PCR experiments were repeated in quadruplicate and are presented above with standard error calculations performed for all conditions.

FIG. 10: Model of protection of Nucleus Pulposus Cells by Notochordal Cell Conditioned Medium (NCCM). (A) Schematic of IL-1β+FasL-mediated changes upon NP gene expression. (B) Changes in NP gene expression changes conferred by NCCM in the presence of IL-1β+FasL. Note that soluble factors secreted by notochordal cells protect the expression of genes encoding extracellular matrix (ECM) proteins, suppresses genes encoding ECM-degrading enzymes and increases gene expression of ECM-protective enzyme TIMP-1. Further, Notochordal cell secreted factors suppress IL-1β+FasL-induced apoptosis.

FIG. 11: Apoptosis and Total Cell death in NP cells treated with basal medium, IL-1β and IL-1β+CTGF. Ten ng/mL rhCTGF suppresses apoptosis of NP cells over 24 hours of incubation. Suppression of total cell death (p=0.03), suppression of apoptosis p=0.009)

FIG. 12: Notochordal cell-rich colonies derived under clonal density tissue culture conditions. (A) Complex cellular constructs generated under hypoxia with elements suggestive of colony formation. (B) Colonies generated under clonal density conditions (×4). (C) Close-up of single colony (×20).

FIG. 13: Anti-aggrecan and anti-collagen type II staining of IVD NP-derived stem cells NCD colony differentiation. (A) anti-aggrecan, (B) anti-collagen type II.

FIG. 14: Neurogenic differentiation of canine IVD-derived and hypoxically generated colonies. (A) anti-beta III tubulin staining (white arrows). (B) anti-GFAP staining of IVD NP-derived stem cells (white arrow).

FIG. 15: Oil Red staining of adipogenesis-driven IVD NP-derived stem cells. The oil-red inclusion droplets are indicated by black arrows.

FIG. 16: Myelin basic protein (MBP) immune-like reactivity within shiverer mouse brain 4 weeks post micro-injection of NCD canine derived colony forming cells. A large, multi-processed cell exhibiting immune-like reactivity to MBP staining (white arrow head) with the (DAPI-labeled) nucleus co-localized within the cell. Serial sections depict the same cell through 3-5 μm thick serial sections. There are no positively labeled cells within any control sections.

FIG. 17: Pluripotential Gene expression of IVD NP cells and colony-derived stem cells. The order of genes examined is identical for lanes 1-8 on the left and right. Amplicons within Lanes 1-8 on the left are developed from cDNA generated from total RNA extracted directly from NCD canine IVD cells. Amplicons in lanes 1-8 on the right are generated from cDNA generated from progenitor cells (Notochordal cell Stem Cell Spheres—NCSC) Lane 1: Oct3/4, Lane 2: Nanog, Lane 3: Sox2, Lane 4; Nestin, Lane 5: BIII tubulin, Lane 6: CD 133, Lane 7 GFAP, Lane 8: HPRT.

FIG. 18: (A) Non-clonal density cell colony formed from notochordal cell-rich nucleus pulposus cells cultured under hypoxic conditions within alginate bead cultures and placed into culture atop glass cover slips within 4-well culture dish. (B) Notochordal-rich nucleus pulposus-derived progenitor cell colony atop matrigel-coated glass coverslip within 4-well tissue culture plate. (C) Image of classic stem cell colony atop feeder cell layer (freely available image public domain).

FIG. 19: (A) Colony derived from notochordal cell-rich nucleus pulposus within alginate bead stained for Beta III tubulin (white arrows); demonstrative of neural precursor cells. (B) Clonal density colony-derived progenitor cells from notochordal cell-rich nucleus pulposus stained for Sox2 expression (white arrows). Cells plated upon glass cover-slips treated with matrigel within 4-well plates. (C) NCAM (neural cell adhesion molecule) staining of notochordal cell-derived colonies (white arrows) formed within alginate beads. These colonies were seen to form after several months in culture within alginate beads cultured with Advanced DMEM/F-12+ between 2 and 8% FBS.

### DESCRIPTION OF VARIOUS EMBODIMENTS

### I. Definitions

The term “a cell” as used herein includes a single cell as well as a plurality or population of cells.

The term “autologous transplantation” as used herein refers a transplant taken and engrafted in the same patient. For example, the method can comprise providing hypoxic compartment cells from a subject, generating in vitro expanded progenitor or differentiated cells comprising or consisting of, for example, neuronal progenitor cells by the methods described herein and transferring the generated progenitor or differentiated cells back into the same subject. The term “non-autologous transplantation” refers to a transplant taken from a subject and engrafted in a different subject. For example, the method can comprise providing hypoxic compartment cells from a subject, generating in vitro expanded progenitor or differentiated cells according to methods described herein and transferring the generated progenitor or differentiated cells back into a different subject.

The term “cell aggregate” as used herein means a 3-dimensional construct, that is obtained for example when hypoxic compartment cells are propagated in hypoxic conditions and that comprises cells and optionally non-cellular components. The cell aggregate can include for example expanded nucleus pulposus (NP) cells such as notochordal cells. The cell aggregates can also comprise other substances such as non cellular components such as such as extracellular matrix components for example collagen type II and aggrecan. The cell aggregates can comprise colony off shoots or “buds”, for example as seen when cells are propagated in alginate globules.

The term “Connective tissue growth factor” or “CTGF” also known as CCN2 as used herein means the gene product of the connective tissue growth factor gene and includes without limitation, CTGF from any species or source, including mammalian CTGF including human CTGF as well as recombinant forms, analogues, for example that are modified to introduce a purification tag, such as a His tag, and fragments or portions of CTGF that maintain CTGF for example the anti-apoptotic activity of CTGF described herein. CTGF is a multi-function molecule considered to be a growth-assistance factor and is a matrix-associated, heparin-binding protein. It may be pro or anti-apoptotic depending upon the cells and conditions and can promote cell adhesion, migration and/or chemotactic functions. CTGF may upregulate the expression of matrix metalloproteinases and the inhibitors of these functions (TIMPS). CTGF deletion in mice is a lethal trait and these animals die soon after birth, secondary to respiratory failure, due to skeletal dysplasia. The CTGF protein may have any one of the known published sequences for CTGF, which can be obtained from public sources such as Genbank. Examples of sequences include Accession EMBL AAH87839.1 and from NCBI, the accession is AAA91279 AAA03285.349 amino acids in length, human CTGF as per J. Cell Biol. 114 (6), 1285-1294 (1991) which are hereby specifically incorporated by reference.

The term “culturing” as used herein means a process by which cells are grown in vitro under controlled conditions, and can include for example maintaining cells, passaging cells, and/or feeding cells.

The term “differentiation” or “differentiated” as used herein refers to the process by which a less specialized cell, such as a stem cell, becomes a more specialized cell type, such that it is committed to a specific lineage.

The term “differentiation culture medium” as used herein means culture medium that permits and/or is optimized cell differentiation. For example, neural differentiation medium is medium that permits and/or is optimized for neural differentiation, chondrocyte differentiation medium is medium that permits and/or is optimized for chondrocyte differentiation and adipocyte differentiation medium is medium that permits and/or is optimized for adipocyte differentiation.

The term “engraftment” as used herein refers to the transfer cells, for example, of cells produced or expanded by the methods described herein, to a subject in need thereof. The graft may be allogeneic, where the cells from one subject are transferred to another subject; xenogeneic, where the cells from a foreign species are transferred to a subject; syngeneic, where the cells are from a genetically identical donor or an autograft, where the cells are transferred from one site to another site on the same subject.

The term “expanded population of cells” as used herein refers to a population of hypoxic compartment cells, and/or a subset thereof, that has been cultured and expanded under hypoxic conditions as described herein. The expanded population can be, for example, further expanded (e.g. serve as a starting population for further expansion, for example in vitro and/or in vivo), differentiated, used in an assay or therapy and/or used to make conditioned media.

As used herein, the term “growth factor” refers to a protein, peptide or other molecule having a growth, proliferative, differentiative, or trophic effect on cells such as stem cell. Proliferation-inducing growth factors include but are not limited to EGF, amphiregulin, acidic fibroblast growth factor (aFGF or FGF-1), basic fibroblast growth factor (bFGF or FGF-2), transforming growth factor alpha (TGFα), and combinations thereof.

The term “hypoxic compartment cells” as used herein means cells found in a hypoxic compartment tissue including but not limited intervertebral disc (IVD) cells, nucleus pulposus cells (NPC) and notochordal cells. Other hypoxic compartment cells include for example, the deep layer of articular cartilage.

The term “hypoxic compartment tissue” refers to tissues such as IVD, nucleus pulposus tissue and the deep layer of articular cartilage. which in vivo exist in compartments where the oxygen concentration is less than, for example 10%, or for example, less than 7%. For example, the oxygen concentration in the deep layer of articular cartilage varies between 7% in more superficial layers and 1% in deeper layers and the oxygen concentration within the healthy IVD NP is reported to vary between 2-5% (82).

The term “in vitro expanded population of cells” as used herein means a population of hypoxic compartment cells and/or a subset thereof that has been isolated and cultured and expanded under hypoxic conditions in vitro.

The term “in vivo expanded population of cells” as used herein refers to a population of hypoxic compartment cells, and/or a subset thereof, that has been expanded in the subject, for example resulting from treatment with conditioned media, and/or CTGF and/or an active fragment of CTGF.

The term “intervertebral disc (IVD) cells” as used herein means any or all of the cells that make up the IVD, including for example NP cells such as Notochordal cells and/or stem/progenitor cells and/or chondrocyte-like cells, annulus cells including annulus fibrous cells.

The term “long term culture” as used herein means culturing for a period of time that is at least 48 hours, at least 1 week, at least 1 month, or at least 2 months. A long-term culture can for example be a continuous culture that is maintained for example for 4 months, 6 months, or 1 year. It can also be a culture that is maintained for a period of time frozen down and restarted, wherein the total culture period at least 1 week, at least 1 month, or at least 2 months.

The term “notochordal cell conditioned medium” or “NCCM” as used herein means culture medium, or an active fraction thereof, comprising soluble factors secreted by notochordal cells cultured under hypoxic conditions for a suitable period of time. Activity of the fraction can be assessed for example by assessing the fraction's ability to inhibit inflammatory cytokine induced apoptosis of NP cells as well as by detecting the upregulation of aggrecan, versican and/or hyaluronic acid synthase. NCCM also is demonstrated to salvages/maintain the expression of one or more of collagaen type II and aggrecan and increases the expression of the CD44 receptor and link protein by NP cells, such that NCCM activity can be assessed by analysis of one or more of the foregoing according to the teachings herein.

The term “nucleus pulposus cells (NPC)” as used herein refers to cells found in nucleus pulposus tissue in a vertebrate, for example a mammal, including but not limited to Notochordal cells and/or stem/progenitor cells and/or chondrocyte-like cells. The cells are optionally referred to as IVD NP cells.

The term “nucleus pulposus conditioned medium” or “NPCM” as used herein means culture medium, or an active fraction thereof, comprising soluble factors secreted by NP cells, or a subset of NP cells, cultured under hypoxic conditions for a suitable period of time. Activity of the fraction can be assessed for example by assessing the fraction's ability to inhibit inflammatory cytokine induced apoptosis of NP cells as well as by detecting the upregulation of aggrecan, versican and/or hyaluronic acid synthase. For example, NPCM, includes NCCM.

The term “nucleus pulposus (NP) tissue” as use herein refers disc tissue found within the disc nucleus pulposus and comprises for example one or more of notochordal cells, chondrocyte-like cells and/or stem/progenitor cells.

The term “neuronal progenitor cell” as used herein means a cell that can give rise to neuronal cells, including but not limited to neurons, glial cells (such as astrocytes) and oligodendrocytes and/or Schwann cells-these are the types of cells that can produce myelin. Oligos are pesent in the CNS and Schwann cells are within the PNS (peripheral nervous system).

The term “oxygen controlled environment” refers to machine such as an incubator, for culturing cells wherein the oxygen level is set or can be set to a desired level, for example at 3.5% oxygen.

The term “oxygen concentration” as used herein means the partial pressure of oxygen a cell is exposed to, for example in vivo and/or in vitro and/or the partial pressure of oxygen dissolved in a fluid or tissue.

The term “passaging” as used herein refers to transferring cells to a new vessel, for example, transferring a portion of the cells in a culture to a new vessel, for example to seed a new culture and/or to expand the culture.

The term “palliating” a disease or disorder means that the extent and/or undesirable clinical manifestations of a disorder or a disease state are lessened and/or time course of the progression is slowed or lengthened, as compared to not treating the disorder.

The term “population of in vitro expanded cells” refers to a population of cells obtained post culture under hypoxic conditions and can comprise notochordal cells, precursor cells and stem cells. The cells can for example be further cultured to obtain more specialized cells such as neural cells.

The term “prevention” or “prophylaxis”, or synonym thereto, as used herein refers to a reduction in the risk or probability of a patient becoming afflicted with degeneration such as disc degeneration or manifesting a symptom associated with degeneration.

The term “progenitor cell” and/or “precursor cell” as used herein means a cell which has the ability of self-renewal, and that can also differentiate into one or more lineages (e.g. a pluripotent progenitor or stem cell), and/or is a less specialized cell that gives rise to a more specialized cell of the same lineage, for example a neuronal progenitor cell gives rise to neuronal cells such as cells that express myelin basic protein. The progenitor cells express progenitor cell markers for example one or more of Beta III tubulin, Nestin, CD133, GFAP, NCAM, Sox2 and/or Oct3/4. Culture conditions for culturing progenitor cells are well known in the art and described for example in Alexanian et al. (83).

The term “spherelike conformations” or “spherelike colonies” as used herein means a cell colony that arises during hypoxic culturing for example with a neural cell optimized medium such as NeuralbasalA™ and that for example, resembles stem cell spheres and/or neurospheres and comprises cells that can differentiate. The spherical conformation can be spherical and or spherelike (e.g. the colony is generally round or oval in, for example round in 3 dimensions when grown in suspension, but reference to “sphere” is not intended to imply a perfectly round 3 dimensional shape).

The term “stem cell” as used herein means a cell that has the ability of self renewal and that can give rise to one or more other cell lineages. Stem cells express for example Sox2, CD133 and/or Oct3/4.

The term “subject” includes all members of the animal kingdom, including human. In one embodiment, the subject is an animal. In another embodiment, the subject is a human.

The terms “transformed”, “transfected” or “transduced” are intended to encompass introduction of a nucleic acid (e.g. a vector) into a cell by one of many possible techniques known in the art. For example, nucleic acid can be introduced into mammalian cells via conventional techniques such as calcium phosphate or calcium chloride co-precipitation, DEAE-dextran mediated transfection, lipofectamine, electroporation or microinjection or via viral transduction or transfection. Suitable methods for transforming, transducing and transfecting cells can be found in Sambrook et al. (Molecular Cloning: A Laboratory Manual, 3rd Edition, Cold Spring Harbor Laboratory Press, 2001), and other laboratory textbooks. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)). For example, a cell can be transformed, transfected or transduced to express CTGF.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term a “therapeutically effective amount”, “effective amount” or a “sufficient amount” of a composition or cell of the present disclosure is a quantity sufficient to, when administered to a cell or a subject, including a mammal, for example a human, effect beneficial or desired results, including clinical results, and, as such, an “effective amount” or synonym thereto depends upon the context in which it is being applied.

The term “under suitable cell culture conditions” as used herein means conditions including for example appropriate temperature gas mixture and nutrient requirements that permit and/or optimize a desired outcome for example that permit optimize cell growth, differentiation, maintenance of progenitor phenotype etc.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The definitions and embodiments described in particular sections are intended to be applicable to other embodiments herein described for which they are suitable as would be understood by a person skilled in the art.

### II. Methods

a. Methods of Maintaining Expanding Cells

It is demonstrated herein that mammalian nucleus pulposus (NP) cells can be maintained and expanded in vitro indefinitely when cultured under hypoxic conditions. NP cells in vivo exist in a hypoxic environment. Accordingly, it is predicted that that the methods described herein are suitable for other hypoxic compartment cells that in vivo exist in a hypoxic compartment tissue.

Accordingly, in an aspect the disclosure includes a method of maintaining and/or expanding an in vitro population of hypoxia compartment cells comprising culturing said population of cells under suitable cell culture conditions, wherein the population of hypoxia compartment cells is exposed to an oxygen concentration of between about 1.5% and about 10%, preferably between about 2% and about 5%. In an embodiment, the cells are cultured in an oxygen controlled environment. In an embodiment, the method further comprises obtaining and/or isolating a population of in vitro expanded cells.

In an embodiment, the population cells comprise intervertebral disc (IVD) cells. In a further embodiment the population of cells comprises nucleus pulposus cells (NPC). In another embodiment, the population of cells comprises notochordal cells. In yet a further embodiment, the population of cells comprises articular cartilage chondrocyte cells.

The cells are optionally primary cells, for example mammalian primary NP cells.

It is demonstrated herein that mammalian NP cells separated from disc annulus, and enzymatically digested to release cells from, for example, surrounding extracellular matrix, can, in addition to being maintained and expanded, develop an organized, complex 3D extracellular matrix rich in collagen type II and aggrecan.

Accordingly, a further aspect of the disclosure includes a method of producing an in vitro population of cell aggregates, for example NP cell aggregates, comprising culturing a population of NP cells under suitable cell culture conditions, wherein the population of NP cells is exposed to an oxygen concentration of between about 1.5% and about 7%, preferably between about 2% and about 5%, for a culture period suitable for producing cell aggregates. In an embodiment, the cells are cultured in an oxygen controlled environment.

In an embodiment, the method further comprises obtaining and/or isolating population of in vitro expanded cell aggregates.

The culture period that is suitable, for example to maintain or expand a population of cells and/or obtain a cell aggregate depends for example on the size and/or number of cell aggregates desired.

The suitable culture period can at least 48 hours, at least 1 week, at least 1 month, at least 2 months, at least 4 months, at least 6 months, or at least 1 year. In an embodiment, the culture is a long term culture.

The conditions the cells are exposed to for example, temperature, culture medium composition will also impact the suitable culture period. For example, it is demonstrated herein, that NP cells can be expanded in the presence or absence of bovine serum such as fetal bovine serum. The cell proliferation rate is higher in cell cultures comprising FBS.

Conditions for culturing should be close to physiological conditions. The pH of the culture medium should be close to physiological pH, preferably between pH 6-8, more preferably between about pH 7 to 7.8, for example pH 7.4. Physiological temperatures range between about 30° C. to 40° C. Cells are preferably cultured at temperatures between about 32° C. to about 38° C., and more preferably between about 35.0° C. to about 37° C.

Any medium can be used that is capable of supporting cell growth, including Advanced DMEM/F-12 and the like, containing supplements which are required for cellular metabolism such as glutamine and other amino acids, vitamins, minerals and useful proteins such as transferring and/or heparin and the like. The medium may also contain antibiotics to prevent contamination with yeast, bacteria and fungi such as penicillin, streptomycin, gentamicin, fungizone and the like. In some cases, the medium may contain serum derived from bovine, equine, chicken and the like. A defined culture medium is also preferred if the cells are to be used for transplantation purposes. In an embodiment, the culture medium is a defined culture medium comprising a mixture of NeuralbasalA™ and a defined hormone and salt mixture. Mediums optimized for specific cells for example NeuralbasalA™ medium which is optimized for neural progenitors, can be used for example with 1% serum when obtaining expanded populations of differentiated cells. Other optimized culture mediums for other cell types are well known in the art.

The culture medium can be supplemented with a proliferation-inducing growth factor(s). In an embodiment, the culture medium comprises EGF and/or FGF. Growth factors are usually added to the culture medium at concentrations ranging between about 1 fg/ml to 1 mg/ml. Concentrations between about 1 to 100 ng/ml are usually sufficient. Simple titration experiments can be easily performed to determine the optimal concentration of a particular growth factor.

In addition to proliferation-inducing growth factors, other growth factors may be added to the culture medium that influence proliferation and differentiation of the cells including NGF, platelet-derived growth factor (PDGF), thyrotropin releasing hormone (TRH), transforming growth factor betas (TGF.beta.s), insulin-like growth factor (IGF-1) and the like. Differentiation can also be induced by growing cells to confluency and/or growing cells on adherent surfaces.

Hormones for example insulin, which is essential for growth of nearly all cells in culture, cortisone, prostaglandin, etc. can also be added particularly when using defined mediums, for example in applications involving transplantation.

The NP cell aggregates have viscoelasctic properties that make the NP aggregates suitable for research studies and trials. Accordingly, in a further embodiment, the population of cells are cultured for a period suitable for the population of in vitro expanded cells to develop viscoelastic properties.

It is demonstrated herein that the cells can be propagated in an alginate gel or bead. Accordingly, in an embodiment, the population of cells is comprised in a biocompatible gel or bead, for example in an alginate gel or alginate bead. In an embodiment, the biocompatible gel comprises an anionic polysaccharide, preferably alginate such as sodium alginate, and a divalent cation salt such as CaCl₂. Other suitable gels include for example agarose gels and other forms of alginate beads formed for example with strontium.

Cells can be cultured in suspension or on a fixed substrate. Alternatively, the cells can be propagated on tissue culture plates or in suspension cultures. Cell suspensions can be seeded in any receptacle capable of sustaining cells, particularly culture flasks, cultures plates, or roller bottles.

The cells can also be propagated on slides, in wells, dishes or flasks. In an embodiment the receptacle for example, the slide, well, dish or flask comprises an adhesive surface. Adhesive surfaces are for example useful in the propagation of cell aggregates, e.g. that resemble NP. Adhesive layers include, without limitation, matrigel, which is a basement membrane analogue. Laminin, fibronectin, collagen (e.g. types I, III and IV) elastin, gelatin vitronectin, fibrin are other examples of adhesive biological materials. Non-biological materials include plasma treated polystyrene. In an embodiment, the cells are propagated on feeder cells for example mouse embryonic fibroblasts (Mefs).

In an embodiment, the oxygen concentration is about 1.5%, about 2% about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%. In an embodiment, the oxygen concentration and culture period selected allows the in vitro expanded population of cells to produce extracellular matrix, produce proteoglycan, and/or produce fibrils and/or form cell aggregates or a sphere-like conformation, optionally oval or crescent shaped, optionally with viscoelastic properties.

In an embodiment, the oxygen concentration selected allows the in vitro expanded population of cells comprised in the biocompatible gel to produce extracellular matrix, produce proteoglycan, produce collagen type II and/or produce aggrecan and/or form a cell aggregate external to the biocompatible gel.

The cells can be maintained continuously under hypoxic conditions, for example cells can be fed in a chamber that maintains oxygen concentration below for example 10%. Alternatively, cells can be exposed to normoxic conditions for short periods of time for example for feeding, counting and/or cell splitting.

In an embodiment, the population of cells is cultured in Advanced DMEM/F-12 culture medium. In an embodiment, the culture medium comprises about 0% to about 10% serum, such as bovine serum, fetal bovine serum etc. In an embodiment, the culture medium comprises about 0%, about 2%, about 4%, about 6%, about 8% or about 10% v/v serum.

It is demonstrated herein that NP cells cultured under hypoxic conditions have an increased cell area compared to normoxic grown cells. In an embodiment, the in vitro expanded population of cells is characterized as having on average at least a 1.5, at least a 1.8, at least a 2.1, at least a 2.4, at least a 2.7 or at least a 3 fold increase in cell area compared to a population of cells exposed to an oxygen concentration of greater than 15%, 17% or 20% (see Example 3).

In another embodiment, the population of in vitro expanded cell aggregates and/or cells comprises progenitor cells, for example neuronal progenitor cells.

It is further demonstrated herein that a subpopulation of NP cells isolated from mammalian spines propagated in vitro form sphere-like conformations and can differentiate. Without wishing to be bound to theory, these cells may trans-differentiate and/or comprise a progenitor subpopulation may be present that can be expanded in vitro.

Accordingly, a further aspect includes a method of generating and/or enriching for hypoxia compartment progenitor cells comprising: providing a population of cells comprising hypoxia compartment cells; maintaining and/or expanding the population of hypoxia compartment cells according to a method described herein for a sufficient time and under suitable conditions for the population of hypoxia compartment cells to form spherelike conformations; and isolating in vitro expanded sphere-like conformations enriched for hypoxia compartment progenitor cells.

In an embodiment, the population of cells to be expanded is isolated from a hypoxia compartment tissue. In an embodiment, the hypoxia compartment tissue is an intervertebral disc, preferably nucleus pulposus tissue and wherein the method of isolating the population of cells comprises isolating nucleus pulposus (NP) tissue, contacting the NP tissue with one or more enzymes, and optionally filtering said cells to obtain the population of cells. For example, the one or more enzymes could include for example, enzymes that release cells from the collagenous network. For example, peptide bonds can be cleaved with pronase and the collagen in the matrix can be digested with collagenases.

Depending on the hypoxic compartment tissue other enzymes and enzyme cocktails targeting other tissue components would be suitable. A person skilled in the art would be familiar with methods for isolating cells from different tissues.

In an embodiment, the cells are plated at 20%, about 30%, about 40% or about 50% confluence. In an embodiment the cells are plated at about 1×10⁶ cells/mL, about 2×10⁶ cells/mL, about 3×10⁶ cells/mL, or about 4×10⁶ cells/mL. For methods relating to progenitor/stem cell cultures, avoiding clumping when seeding or passaging cells can increase confidence that the colonies obtained arise from a single cell. Accordingly, in an embodiment, the population of cells comprising progenitor cells are plated at a concentration that is below clonal density to avoid clumping of cells. In an embodiment, cells are seeded or plated between about 2000 cells/mL and about 10 000 cells/ml. In another embodiment, the cells are seeded or plated at less than about 8000 cells/mL, less than about 6000 cells/mL at less than about 5000 cells/mL. In a preferred embodiment, stem and progenitor cells are cultured in suspension cultures.

The in vitro expanded progenitor cells can be further enriched by isolation and resuspending the sphere-like conformations that are enriched for progenitor cells. In an embodiment, isolating the in vitro expanded spherelike conformations comprises centrifuging the spherelike conformations.

For example, the progenitor cells are in an embodiment grown within ultra-low adherence tissue culture flasks in suspension culture which allows the cells to form the sphere like conformations s in suspension. In an embodiment, expanding the progenitor cells or spherelike conformations involves dispersing the cells in the spherelike conformation using for example trypsin/EDTA (commercially supplied-invitrogen) for a suitable period of time, for example 2-5 minutes with gentle pipetting in order to break-up the spheres. In an embodiment, the cells are centrifuged and washed with trypsin inhibitor within neural basal A (1 mg/mL), centrifuged again (500×G) and recovered and re-suspended at clonal density.

Isolated ex vivo expanded cell aggregates and/or multilayer sphere-like conformations, for cells re-suspended in a suitable culture medium are in an embodiment, plated on a culture vessel such as a dish, flask or cover slip that comprises an adhesive surface, such as adhesive coated cover slips such as Matrigel™ coated cover slips.

In another embodiment, isolating the cell aggregates and/or sphere-like conformations comprises for methods wherein the cells are suspended in the biocompatible gel, dissolving the gel, for example with sodium citrate, separating the cells for example by density gradient separation, and recovering layers comprising the cell aggregates and/or spherelike conformations. For example, density gradient layers that correspond to the 1.014 and 1.021 specific gravity levels of a Percoll density gradient developed in 1 mL volumes for example, at 1.007, 1.014, 1.021, 1.028 and 1.035 specific gravity steps, centrifuged at 200×G for 20 minutes, are enriched for cell aggregates and/or spherelike conformations. For example, other gradients include Ficoll gradient. Flow cytometry can optionally used to separate cells for example based on cell size. A person skilled in the art would be familiar with other cell separation and enrichment techniques and would on the basis of the teaching herein readily be able to separate and/or enrich for the desired cells.

In an embodiment, the method further comprises culturing the recovered layers, according to a method described herein, for example by resuspending the recovered layers, or cells comprised therein, in a biocompatible gel and culturing for a suitable period for example about 3 weeks, about 4 weeks, about 5 weeks or about 6 weeks.

It has been demonstrated herein, that the progenitor cells express one or more progenitor cell markers. Accordingly in an embodiment, the progenitor cells express one or more of Beta III tubulin, Nestin, GFAP, NCAM, Sox2 and/or Oct3/4 and CD133. Progenitor cells can be confirmed for example by detection of one or more of these markers. Expression of one or more of these markers can be detected by methods known in the art, including for example immunohistochemistry and RT-PCR, including quantitative and multiples PCR and in the case of cell surface markers such as nestin, fluorescence activated cell sorting.

It is demonstrated herein that the progenitor cells can differentiate to neuronal cells. Accordingly, in an embodiment, the in vitro expanded population of cells comprises progenitors that can give rise to neuronal cells e.g. neuronal progenitor cells. Specifically, it is demonstrated that neuronal progenitor cells are capable of differentiating to neuronal cells expressing myelin basic protein in vivo. Accordingly, in an embodiment, the method comprises producing neuronal progenitor cells. In an embodiment, the hypoxia compartment cells that are expanded comprise NP cells. In an embodiment, conditions are optimized for producing spherelike conformations. In an embodiment, NP cells are cultured in medium which is optimized for stem cell culture and/or neural progenitor cell culture. In an embodiment, the culture medium is Neuralbasal A™ medium supplemented for example with hormones, growth factors and/or PSF. In an embodiment, the conditions comprise using ultra-low adherence tissue culture flasks, for example CoStar™ cell culture plates available from Corning™.

Sphere-like colonies are visible for example after 10 days of culture depending on the culture conditions and are collected in an embodiment by inversion filtering. Cells can be replated for further expansion, until a suitable expansion is obtained.

In an embodiment, the NP cells are cultured for a period of at least 8 days, 9 days, 10 days, 11 days, 12 days, 13 days, 14 days or 15 days, at least 2 weeks or at least 3 weeks under suitable conditions to produce neuronal progenitor cells.

The percent of progenitor cells in the spherical like colonies was investigated and found for example to be about 1% under the conditions used (see for example 5). In an embodiment, the percent of progenitor cells in the cell aggregates and/or sphere-like conformations is at least 0.2%, at least 0.3%, at least 0.4%, at least 0.5%, at least 0.6%, at least 0.7% at least 0.8% at least 0.9% or at least 1.0% progenitor cells, for example neuronal progenitor cells.

The lineage capacity of the progenitor cells was also investigated. It is demonstrated herein that the sphere-like colonies can be directed to differentiate along specific cell lineages. Specifically it is demonstrated herein that the sphere-like colonies can be induced to differentiate along chondrocyte, neural and adipogenic lineages.

Accordingly, a further aspect is an in vitro method of differentiating cells comprising obtaining spherelike conformations, culturing the spherelike conformations in a differentiation culture medium under suitable conditions to obtain differentiated cells.

In an embodiment, the differentiation is in vitro. In another embodiment, the differentiation is in vivo.

In an embodiment, the suitable conditions for differentiation comprise culturing at normoxic oxygen tension. In an embodiment, the suitable conditions for differentiation comprise an oxygen tension of at least 17%, at least 18%, at least 19%, at least 20% or at least 21%.

In an embodiment, the differentiation culture medium comprises neural differentiation culture medium. In an embodiment, the differentiation culture medium comprises chondrocyte differentiation culture medium. In an embodiment, the differentiation culture medium comprises adipocyte differentiation culture medium. In an embodiment, the differentiation culture comprises osteogenic differentiation culture medium. In an embodiment, the differentiation culture medium comprises for example cardiomyocyte, fibroblast and/or tenocyte differentiation culture medium. In an embodiment, neural differentiation is induced using Neural basal A medium supplemented with 1% FBS without the addition of EGF and FGF.

A person skilled in the art would be familiar for example with methods for differentiating cells into different cell types for example neural, chondrogenic, and/or adipocyte cell types.

In and embodiment, the population of cells comprises notochordal cells, preferably primary notochordal cells. In an embodiment, the cells are mammalian. In another embodiment, the cells are canine. In an embodiment, the cells are derived from a non-chondrodystrophic (NCD) canine. In a further embodiment, the cells are human. The cells can be obtained from donor tissue, by dissociation of individual cells from the connecting extracellular matrix of the tissue. Tissue is removed, for example using a sterile procedure, and the cells are dissociated using any method known in the art including treatment with enzymes such as trypsin, collagenase and the like.

For example, the cells can be obtained from surgical interventions where a disc is being replaced. For example, a patient with a herniated disc may require a discectomy. In such a procedure, tissue is harvested and discarded and/or sent for analysed by pathology. The removed tissue can for example be used in the methods described herein, for example to obtain expanded populations of cell aggregates and/or sphere-like conformations. Expanded cells can then be re-implanted.

Neuronal progenitor cells can be propagated in culture medium optimized to develop neural stem cells. In an embodiment, the method further comprises culturing the cells in a neural stem cell culture medium, for example Neural basal A™ medium. In an embodiment, the culture medium optionally comprising hormone mix, EGF, FGC and heparin.

Other factors such as antibiotics, fungicidal agents and/or other nutrients that are well known in the art, can also be included in the culture medium.

In an embodiment, the method can further comprise incorporating isolated in vitro expanded cells, into a scaffolding comprising matrix. For example, cells can be incorporated via electrospinning/electrospraying cells such as notochordal cells within a solublized matrix such as type II collagen and after suitable preparation the mixture of solublized carrier and cells are subjected to an electrical field and small bore nozzle the cell/carrier-matrix is fabricated into a 3-D matrix encompassing the cells. An appropriate matrix includes for example, type II collagen, polyglycolic acid, or other biologically suitable polymer.

In the case of progenitor cells, a suitable scaffolding could be fabricated such as via electrospinning also containing suitable cells such as chondrocytes, neural or other suitably directed cell lineage.

b. Assay Methods

The NP cell aggregates and/or sphere-like conformations have viscoelastic properties that make the cell aggregates and/or sphere-like conformations suitable for research studies and trials.

Accordingly, a further aspect of the disclosure includes a method for testing sensitivity of a population of hypoxia compartment cells to a test agent or combination, comprising maintaining and/or expanding an in vitro population of hypoxia compartment cells according to a method described herein; adding the test agent or combination and determining sensitivity of said cells to therapeutic test agent or combination. The sensitivity can be determined for example by comparing to a control, for example an untreated population of cells.

The methods and cells described herein may be used for the study of the cellular and molecular biology of NP or NP cell development, for the discovery of genes, growth factors, and differentiation factors that play a role in progenitor cell differentiation and for drug discovery.

In an embodiment, sensitivity is determined by quantifying cell death, inhibition of cell death and/or proliferation. Gene expression, for example detecting gene expression of progenitor or cell type specific markers, can also be used to determine sensitivity of the cells. In an embodiment, the expression of one or more of aggrecan, link protein, CD44 receptor, collagen type II, MMP3, TIMP1, and ADAMTS4 are detected.

In an embodiment, the test agent is a chemical or other substance, such as a drug, being tested for its effect on the differentiation of the cells into specific cell types. In such an embodiment, the analysis may comprise detecting markers of differentiated cell types. In another embodiment, the test agent is a chemical or drug and the screening is used as a primary or secondary screen to assess the efficacy and safety of the agent.

For example, biologically based therapies for treating DDD can be tested in a biologically relevant model.

c. Therapeutic Methods

i. Cells

Cells maintained and/or expanded using the methods described herein are useful for generating cells, including progenitor cells that are useful for treating diseases such as degenerative disc disease (DDD).

Accordingly, in another aspect, the application includes a method of producing a therapeutic hypoxia compartment cell comprising isolating a hypoxia compartment cell and maintaining and/or expanding said cells according to a method described herein under conditions suitable for therapeutic applications. For example, cells propagated for therapeutic applications require that cultures be for example pathogen free.

ii. Conditioned Media and CTGF

It is demonstrated herein that cells propagated using the methods described herein produce soluble factors that are useful for inhibiting inflammatory cytokine and death receptor signaling of NP cells.

Accordingly, a further aspect of the disclosure includes a method of producing nucleus pulposus cell conditioned medium (NPCM) comprising culturing a population of nucleus pulposus cells for example comprising notochord cells, preferably primary notochordal cells, in cell culture medium under suitable cell culture conditions, wherein the population of NP cells is exposed to an oxygen concentration of between about 1.5% and about 7%, preferably between about 2% and about 5%, for a suitable culture period, thereby producing NPCM. In an embodiment, the method comprises culturing a population of notochordal cells thereby producing notochord conditioned medium (NCCM) In an embodiment, the cells are cultured in an oxygen controlled environment. In an embodiment, the culture medium is a basal culture medium devoid of serum and/or growth factors. In an embodiment, the notochordal cells are cultured for a culture period of at least 12 hours, at least 24 hours, at least 36 hours, at least 48 hours, at least 60 hours or at least 72 hours.

In an embodiment, the culture medium comprises Advanced DMEM/F-12, DMEM/F-12. Other suitable culture mediums are known in the art and include for example, DMEM or any other basal supportive tissue culture medium.

As described elsewhere, the cells in an embodiment are comprised in a biocompatible gel.

In an embodiment, the cells are seeded at a concentration of about 2×10⁶ cells/mL to about 4×10⁶ cells/mL, for example per mL of biocompatible gel, for example alginate.

The NPCM or NCCM can be utilized directly and can comprise cells or be cell free. In an embodiment, the method further comprises separating the notochordal cell conditioned media from the notochordal cells.

In an embodiment, the NPCM and/or NCCM is fractionated, for example to isolate a fraction containing factors less than about 80 kDa, less than about 70 kDa, less than about 60 kDa, less than about 50 kDa, or less than about 40 kDa.

Cells maintained and expanded as described herein as well as the conditioned medium described are useful for inhibiting cell death and treating degenerative diseases.

It has further been demonstrated that the growth factor CTGF which is secreted by notochordal cells in culture, exhibits similar inhibition of inflammatory cytokine and death receptor death signaling in NP cells.

Accordingly, a further aspect of the disclosure includes a method of inhibiting nucleus pulposus cell death comprising contacting a nucleus pulposus cell that is being exposed or will be exposed to a cell death signal with the NC conditioned media described herein and/or contacting the nucleus pulposus cell with CTGF, preferably recombinant CTGF, more preferably recombinant human CTGF (rhCTGF). In an embodiment, the NP cell that is that is being exposed or will be exposed to a cell death signal is contacted with a cell that produces CTGF. In an embodiment, the cell is optionally genetically engineered to express exogenous CTGF

In an embodiment, the cell death signal comprises cytochrome C, inflammatory cytokine, for example IL-1b, and/or death receptor mediated, for example FasR, cell death.

A further aspect includes a method of inhibiting inflammatory cytokine and death receptor induced gene expression modulation in a NP cell comprising contacting the NP cell with the notochordal cell conditioned medium (NCCM) described herein, CTGF or an isolated cell of described herein, wherein the matrix gene is selected from aggrecan link, CD44 receptor, collagen type II, MMP3, TIMP-1, wherein contact between the NP cell and the NCCM, CTGF or isolated cell inhibits matrix gene expression modulation induced by inflammatory cytokines.

The cells conditioned medium and CTGF are useful for therapeutic treatments of a subject.

Accordingly a further aspect includes a method of inhibiting nucleus pulposus degeneration in a subject comprising administering, or contacting a population of the subject's NP cells, with a notochordal cell conditioned medium (NCCM) described herein, CTGF or an isolated cell described herein.

In an embodiment, the subject has mild degenerative disc disease (DDD) or an acutely herniated IVD and is administered NCCM or CTGF.

For example, a subject with a herniated disc may require for example a discectomy, where, tissue is harvested. The harvested hypoxic cell compartment tissue is subjected to the methods described herein, for example to obtain expanded populations of cell aggregates and/or spherelike conformations Expanded cells are for example re-implanted in the subject. The subject is optionally monitored for example for mobility gains, and/or reduced pain.

In another embodiment, the subject has advanced DDD, for example DDD requiring surgical intervention, and is administered an isolated cell described herein, optionally at the time of surgery.

Also included in an aspect is a method of treating degenerative disc disease (DDD) in a subject comprising administering notochordal cell conditioned medium (NCCM) described herein, CTGF or an isolated cell described herein to a subject in need thereof.

A further aspect includes use of condition notochordal cell conditioned media (NCCM), CTGF or an isolated cell described herein for treating DDD.

In an embodiment, the treatment inhibits ECM degradation.

A further aspect is a Notochordal cell conditioned medium (NCCM), CTGF or an isolated cell described herein for treating DDD and/or inhibiting ECM degradation.

In an embodiment, the concentration of CTGF administered is from about 0.1 ng/mL to about 1 ng/mL, from about 1.1 ng/mL to about 10 ng/mL, from about 11 ng/mL to about 50 ng/mL, from about 51 mg/mL to about 100 ng/mL. In another embodiment, the CTGF administered is from about 101 ng/mL to about 200 ng/ml, from about 201 ng/mL to about 300 ng/mL.

It is demonstrated herein that the progenitor cells that express neuronal markers can in vivo differentiate to myelin basic protein expressing cells. The shiverer mouse represents an accepted animal model of diseases involving myelination defects. Accordingly a further aspect includes a method of treating a neuro-degenerative disease.

Examples of neurodegenerative diseases that may be treated include for example demyelinating and dysmyelinating diseases, such as multiple sclerosis, transverse myelitis, Devic's disease, progressive multifocal leukoencephalopathy, optic neuritis, leukodystrophies, Guillain Barre syndrome, chronic inflammaroty demyelinating polyneuropathy, anti-MAC peripheral neuropathy, Charcot-Marie Tooth disease as well as Alzheimer's disease, Parkinson's disease, Huntington's disease, Bell's palsy, Pick's disease and amyotrophic lateral sclerosis. In addition, the cells and compositions can for example be used to spinal cord injury. For example a subject with a spinal injury whereby a disc is damaged maybe a suitable candidate to harvest progenitor cells from the subject's discs. Progenitor cells are then expanded and optionally differentiated and introduced for example with CTGF and monitored for evidence of spinal cord repair.

Within the context of hypoxically-derived complex 3D tissue constructs that stain for collagen type II and aggrecan, these cells and the ECM that they produce is consistent with the recapitulation of the IVD NP ‘matrix’.

Also provided herein is a method of engraftment comprising transferring the cells described herein to a subject in need thereof. In an embodiment, the population of cells is autologous. In another embodiment, the population of cells is allogeneic. In an embodiment, the method of engraftment comprises autologous transplantation. In a further embodiment, the method comprises non-autologous or allogeneic transplantation.

For example, hypoxia compartment cells are in an embodiment, harvested from a subject expanded according to a method described herein and the in vitro expanded population is reintroduced into the subject (e.g. autologous engraftment) or a different subject (e.g. allogeneic engraftment).

In an embodiment, the method of administering or contacting comprises a percutaneous delivery of this factor, delivery at the time of surgery such as micro-discectomy in the form of injection and/or the application of a time-delayed delivery of CTGF/CCN2 within the disc or other controlled delivery methods.

In an embodiment, the population of hypoxic compartment cells, for example NP cells are expanded to form complex 3D constructs cell aggregates which as is demonstrated herein resemble NP. In an embodiment, the cell aggregates are used to replace a degenerate or diseased disc in a subject.

In an embodiment, the in vitro expanded cells, CTGF NPCM and/or NCCM is comprises in a composition.

In an embodiment, the in vitro expanded cells, CTGF, NPCM, NCCM and/or compositions described herein are administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

### III. Cells and Compositions

a. Cells

A further aspect of the disclosure includes an isolated cell. In an embodiment, the cell is an isolated hypoxia compartment cell isolated from a hypoxia compartment tissue maintained and/or expanded according to a method described herein. In another embodiment, the cell is an in vitro expanded cell. In a further embodiment, the cell is an in vitro expanded progenitor cell.

In an embodiment, the hypoxia compartment cell is a NP cell. In an embodiment, the in vitro expanded cell is derived from a NP cell.

Also provided is an isolated population of cells. In an embodiment, the isolated population of cells comprises hypoxia compartment cells isolated from a hypoxia compartment tissue, wherein the population of cells is maintained and/or expanded according to a method described herein. In another embodiment, the population of cells is a population of in vitro expanded cells. In a further embodiment, the population of cells is a population of in vitro expanded progenitor cells.

In an embodiment, the method of maintaining or expanding, comprises culturing the cells for at least 48 hours, at least 1 week, at least 2 weeks, at least 1 month, at least 2 months, or longer.

A further aspect of the disclosure provides an isolated differentiated cell, wherein the cell is maintained and/or expanded according to a method described herein and differentiated using differentiation culture medium. In an embodiment, the differentiated cell expresses neuronal cell markers for example myelin basic protein, GFAP and/or beta III tubulin. In an embodiment, the isolated differentiated cell is a neuronal cell lineage cell. In another embodiment, the isolated differentiated cells expresses chondrocyte markers such as aggrecan and/or collagen type II. In another embodiment, the isolated differentiated cell is a chondrocyte cell. In another embodiment, the isolated differentiated cell comprises lipid droplets within the cell, for example detectable by oil red staining. In a further embodiment, the isolated differentiated cell is an adipocyte cell. In another embodiment, the isolated differentiated cell expresses osteogenic markers, such as alkaline phosphatase, and positive Von Kassa and/or Alizarin Red staining. In yet a further embodiment, the isolated differentiated cell is an osteogenic cell. In a further embodiment, the disclosure provides an in vitro expanded population of differentiated cells. In an embodiment, the isolated population of differentiated cells comprises one or more of neural lineage cells, chondrocytes, adipocytes, osteogenic cells and/or precursor cells.

In an embodiment, the cell is incorporated within an appropriate scaffolding. For example, cells can be incorporated via electrospinning/electrospraying cells such as notochordal cells within a solublized matrix such as type II collagen and after suitable preparation the mixture of solublized carrier and cells are subjected to an electrical field and small bore nozzle the cell/carrier-matrix is fabricated into a 3-D matrix encompassing the cells. An appropriate matrix includes for example, type II collagen, polyglycolic acid, or other biologically suitable polymer.

Accordingly, in an embodiment, the isolated cell is comprised in a scaffolding comprising matrix, for example wherein the matrix comprises type II collagen, and/or polyglycolic acid.

The cellular/scaffold may then be implanted within the target tissue such as the intervertebral disc. In the case of progenitor cells a suitable scaffolding could be fabricated such as via electrospinning also containing suitable cells such as chondrocytes, neural or other suitably directed cell lineage.

These cells can be used for example in therapeutic applications, in assays for testing for example, new therapeutic or differentiation agents.

In an embodiment, the cells are comprised in a composition, for example with a suitable diluent.

b. Conditioned Medium

The disclosure further describes making a conditioned medium produced in a hypoxic culture environment

Accordingly, a further aspect of the disclosure includes a composition of notochordal cell conditioned medium (NCCM) wherein the NCCM is produced according a method described herein, for example the method comprising in an embodiment, culturing a population of notochordal cells, preferably primary notochordal cells, in a cell culture medium under suitable cell culture conditions, wherein the population of notochordal cells is exposed to an oxygen concentration of between about 1.5% and about 10%, preferably between about 2% and about 5%. The NC cells can be cultured according to any method described herein. In an embodiment, the cells are cultured in an oxygen controlled environment.

In an embodiment, the culture medium is preferably basal culture medium devoid of serum and/or growth factors, for a suitable culture period. In an embodiment, the suitable culture period is, for example of at least 12 hours, at least 24 hours, at least 36 hours, at least 48 hours, at least 60 hours or at least 72 hours.

In another embodiment, the NCCM is fractionated, for example to isolate a fraction containing factors less than about 80 kDa, less than about 70 kDa, less than about 60 kDa, less than about 50 kDa, or less than about 40 kDa.

The composition in an embodiment comprises NPCM and/or NCCM and a suitable diluent.

c. CTGF

Yet a further aspect comprises a composition comprising CTGF and optionally a carrier, such a pharmaceutical composition. The composition is useful for example in the therapeutic methods described herein. In an embodiment, the composition comprising CTGF is for hypoxic use. In an embodiment, the composition is for the use of CTGF within an the IVD CTGF is known to be affected by TGF-B signaling in many cells and systems. Accordingly, in an embodiment, the composition further comprises recombinant TGF-b

The compositions for example comprising, CTGF, NCCM and/or the in vitro expanded cells are suitably formulated into pharmaceutical compositions for administration to human subjects in a biologically compatible form suitable for administration in vivo.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

Suitable vehicles are described, for example, in Remington's Pharmaceutical Sciences (2003-20^{th} Edition). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The following non-limiting examples are illustrative of the present application:

### EXAMPLES

### Example 1

### Introduction

Degenerative disc disease (DDD) is an extremely common and expensive healthcare ailment which unlike advances in biologic therapies for fracture management and disease modifying drugs for various arthropathies has no curative strategy which attenuates or reverses the degenerative cascade⁹. With respect to the pursuit of a biological intervention the study of the non-chondrodystrophic canine (NCD canine) is noteworthy in that this animal is unique amongst the canine sub-species in that these dogs preserve their notochordal cell populations and are protected from the development of degenerative disc disease (DDD)^{2,4}. It was previously determined that non-chondrodystrophic canine intervertebral discs-derived notochordal cells secrete Connective Tissue Growth Factor (CTGF/CCN-2) and that conditioned medium obtained from these cells up-regulates important matrix gene expression, cell proliferation and proteoglycan production in nucleus pulposus (NP) cells^{7,8}. CTGF is known to be an important regulatory molecule in development that works in conjunction with a number of transcription factors and other developmental genes such as jun, sox5,6,9, hedgehog, and noggin, important genes in disc formation and chondrogenesis^{3,5}.

The intervertebral disc is a highly avascular compartment that is almost totally devoid of vascular supply in the mature adult. Therefore nutrient balance in the IVD is likely tightly regulated by both the cells within and structural aspects of the disc including the vertebral endplates. The importance of nutrient and gas diffusion to the disc (a structure otherwise devoid of vasculature) has been highlighted by a number of studies^{11,19,25,26}. It has been reported that the capillary networks present at the vertebral endplates are up to four times more dense directly over the disc nucleus as compared to the annulus-suggestive of the vital supply of such nutrients to disc cells in the absence of direct vascular supply^{25,26}. Nucleus pulposus cells (NPC) have necessarily adapted to survive within a hypoxic and relatively nutrient-poor environment, the oxygen concentration within this compartment reportedly being between 2-5% [9]. However most studies concerning intervertebral disc metabolism within the in vitro setting have studied these cells cultured under classical tissue culture conditions of 21% O₂^{2,7,8,13,22,23,27,29}. A number of reports concerning the delivery of some of these factors have claimed effective restoration of disc height in vivo and/or increased expression of desirable genes such as aggrecan and collagen. However the tissue culture periods used are often only a matter of days and the cultures are also often maintained within monolayer. Further, nucleus pulposus cells assume a fibroblastic phenotype in monolayer culture-very different from that found in the in vivo setting^{22,24,28}.

In order to develop effective future biological therapeutics that may involve gene transfer, the injection of growth factors and/or stem-cell based therapy it is vital that such interventions be designed and studied in a biologically relevant system. Therefore it is critical to determine the tissue culture conditions under which such critical applications must be performed and to our knowledge have never before been examined in detail-particularly with respect to notochordal cells-cells vital to the developmental organization of the disc nucleus.

The realities of the hypoxic, ischemic environment within the disc nucleus create a unique challenge to the investigator to draw conclusions from in vitro experimentation that is biologically credible and biologically relevant. There are a number of potential biologically-based therapies currently under development and reported in the literature^{15,22,23}. However the studies that reflect in vitro experimentation have been largely performed under classical tissue culture oxygen saturation-a condition that does not mirror the in vivo condition. Further the development of a biologic therapy to treat DDD requires that preliminary studies must be performed within the context of biologically relevant experimental approaches with an emphasis upon primary cells. Primary cells reflect biological realities that may not be the case when using cell lines obtained by immortalization. The biology of primary disc-derived notochordal cells is found to be particularly interesting since it is these cells that provide an anabolic/matrix protective function upon NP cells [4, 5]. Therefore, in this study evaluating the ability of primary notochordal cells to survive under long-term culture, examining the quality of the matrix that they produce and comparing these variables with traditional normoxic tissue culture was chosen.

The healthy disc nucleus maintains a range of O₂ concentration of between 2-5%, therefore for our hypoxic studies a mid-range value of 3.5% was chosen. During pilot studies it was found that maintaining 2% O₂ levels though certainly possible required an inordinate usage of nitrogen gas in order to displace the O₂. Hypoxic responsive genes are active below 5% O₂ therefore 3.5% satisfied both the requirements of hypoxic culture and did not consume nitrogen at a prodigious rate.

It is reported herein that primary notochordal cells cultured under hypoxic conditions thrive and form complex 3D tissue and remain viable for at least 5 months.

### Methods and Materials:

### Non-Chondrodystrophic Canine Disc Notochordal Cell Cultures

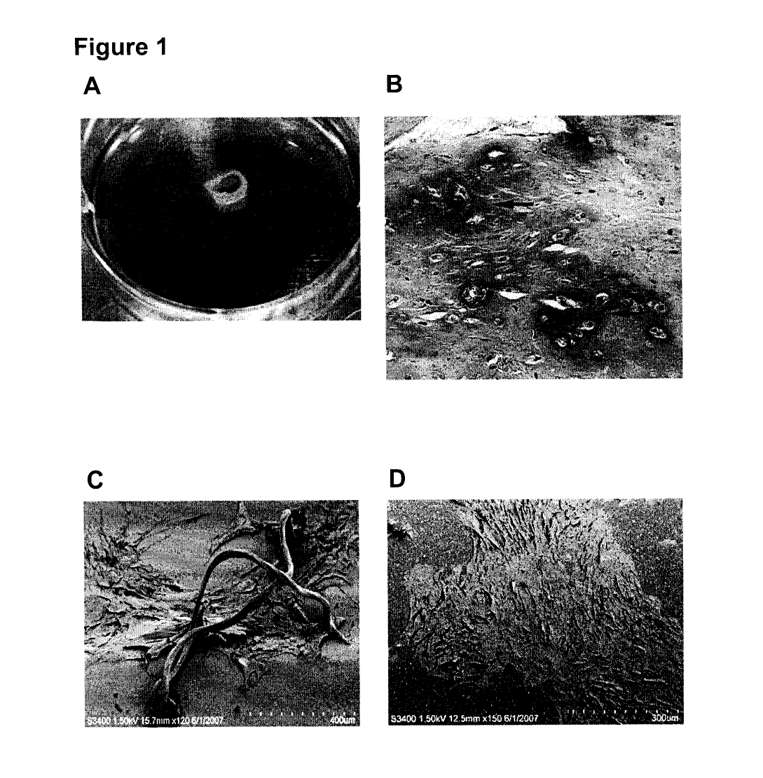

Notochordal cells were obtained by meticulous dissection of the nucleus pulposus from five freshly sacrificed non-chondrodystrophic canine lumbar spines (unclaimed animals that had failed at adoption). Within 2 hours of euthanasia the lumbar spines were removed, the spinal soft tissues dissected away and the spines were washed with DH₂0, Clidox™, and then liberally rinsed with Betadine™ and cooled to 4° C. The discs were placed into a 70 mm² Petri dish and suspended within Advanced DMEM/F-12 supplemented with penicillin, streptomycin and fungizone (PSF) and 5% fetal calf serum (FCS) and placed into 2% O₂ (FIGS. 1 A-D) or 3.5% (remaining experiments) O₂ and 5% CO₂. The following day the nuclei pulposi were meticulously separated from any remaining annulus and washed three times with medium. Next the isolated nucleus pulposus tissues were sequentially enzymatically digested according to our pre-established methods⁷. The following day the cells were rinsed, filtered through 70 μm cell strainers (Falcon) and examined microscopically. Notochordal cells obtained in this fashion produced a virtually pure population of notochordal cells and density gradient separation techniques were not required. The cells were then counted and washed three times with medium and then centrifuged at 500×G. The cell pellet was then mixed with 1.2% sodium alginate at a concentration of 3×10⁶ cells/mL. 0.75 mL of the alginate/cell suspension was pipetted into each well of a 6-well plate containing 102 mM CaCl₂ to form an oval alginate ‘globule’. The globule was allowed to polymerize for 15-20 minutes and was then rinsed three times with PBS (pH 7.4) and finally replaced with complete DMEM/F-12 advanced medium supplemented with 8% FCS and PSF (100 U/mL Penicillin, streptomycin, fungizone) and the medium replaced each three days throughout the culture period. Other notochordal cells were removed and plated directly into 6-well plates at a density of 0.5×10⁶ cells/well and complete medium added to the wells. The ‘globule’ and monolayer cultures were then inserted into either a standard incubator set to 37° C., 5% CO₂ and 21% O₂ or a hypoxic incubator set to 37° C., 5% CO₂ and 3.5% O₂ (Nuaire) and were monitored each two days and followed over selected time periods for up to 5 months under both normoxia and hypoxia. The cells cultured under hypoxic conditions were maintained under hypoxia during feeding by transferring the cultures to a glove box set to the identical conditions of hypoxia (Coy). The cultures were photographed each two weeks as they were observed over time.

In order to compare ‘native’ intervertebral disc nucleus pulposus and the hypoxic and normoxic cultured cells IVD's were obtained from the same animals that were used for the source of notochordal cells in all other experiments by removing the underlying vertebrae complete with the attached IVD. Therefore the specimens were harvested directly from dissection fixed them in 4% paraformaldehyde and decalcified in EDTA for one week and then embedded the specimens in paraffin. These specimens were then sectioned at 4 μm, mounted on cover slips and stained using Safranin-O for histomorphometric analysis (see below for details) and for immune-like reactivity to anti-collagen II and aggrecan.

### Histological Analysis of Notochordal Cells:

At the appropriate time points some of the alginate globules were removed, fixed and embedded in paraffin, sectioned and stained. Briefly, fixation of the globules consisted of using 0.1 M Cacodylate (Sigma) in 10 mM CaCl₂ and 4% paraformaldeyde ph 7.4. The globules were left a room temperature for 8 hours and then switched to 0.1M Cacodylate buffer containing 50 mM BaCl₂ pH 7.4 and changed 4 times over 16 hours and kept at 4° C. The globules were then removed from buffer and embedded in paraffin sectioned, dehydrated through alcohols and xylenes stained and cover-slipped. Five months of culture was chosen as our endpoint in order to ascertain the behaviour of the cultures over long-term conditions. At 5 months, then alginate globules containing notochordal cells were fixed using cacodylate buffer modified from Petit et al 1996 and processed for histological examination and stained with Toluidine Blue, Safranin-0, and for immuno-reactivity to collagen type II (Chemicon MAB1330) and aggrecan (Abcam BC-13 clone, ab3775).

### Cell Morphometry:

Histomorphometric methods to evaluate the cultured cells were used. Total cell area measurements were performed using a Visiopharm Integrator System (Visiopharm Inc. Horsholm-Denmark) using a Leica DM 4500 B microscope equipped with a motorized stage with 8-slide capacity and Olympus DP70 camera. 100 cells from Safranin-O stained sections of both normoxic and hypoxic cultured cells were counted using specific inclusion criteria that required the cells to have an intact cell membrane, visible nucleus and clear cytoplasm²¹. Each cell was individually masked and image analysis was performed to obtain total cell area. Visiopharm™ software was used for the analysis of cell area.

### Scanning Electron Microscopy:

Parallel notochordal cell cultures were grown in alginate globules removed, fixed and prepared for electron microscopy. Briefly, the alginate globule containing cells were immersed in primary fixative containing 4% Paraformaldehyde, 1% Glutaraldehyde in 0.1 M phosphate buffer pH 7.2 for 20 minutes. Next they are washed with 0.1 M Phosphate buffer (pH 7.2) 3 times and post fixation was performed using 1% Osmium Tetraoxide buffered with 0.1 M Phosphate PH 7.2 for 20 minutes and dehydrated through graded alcohols and dried using a critical point drier. The fixed globules were examined using Scanning Electron Microscopy (Hitachi S-3400 Variable Pressure Scanning Electron Microscope) at various magnifications and angulations of the stage in order to obtain the best imaging of the specimens.

### Immunohistochemistry:

Specimens were sectioned at 4 μm, mounted on silanized slides cleared using xylenes and alcohols and stained in parallel with positive and negative controls. The antibodies were optimized for concentration of primary antibody such that anti-collagen II was stained at 1:800. The anti-aggrecan was obtained from a hybridoma supernatant and therefore was not as concentrated and was used at a 1:10 dilution. The immune-like reactivity was evaluated using avidin-biotin conjugated primary and secondary antibodies, visualized using diamino-benzidine methods (DAB kit-vecta stain) and counter stained with haematoxylin. For positive control, articular cartilage, known to contain both aggrecan and collagen II (bovine tarsal joints) was stained and the negative control was absent the primary antibody.

### Statistical Analysis

The data obtained from our histomorphometric analysis which consisted of 200 separate measurements was plotted and as suspected found to be not normally distributed. Therefore non-parametric statistical methods were used to analyze for statistical significance. The Mann-Whitney U test was used and it was found that the mean cell area for hypoxic cultured as compared to normoxic cultured cells was statistically significant at the P=0.0001 level.

### Results:

Hypoxic and Normoxic Monolayer Cultures:

Canine non-chondrodystrophic notochordal cells thrive under hypoxic culture for at least 5 months, the time at which they were removed from culture and evaluated. There was no evidence of decline in cell growth within the hypoxic cultures at 5 months. The cultures were examined each three days as the medium was replenished and were photographed at least weekly throughout the 5 months of culture. Within one week cells appeared upon the bottom of the E-well dishes initially as spherical/ovoid bubbly looking or “physaliferous” cells. Within two-weeks these cells had spread to become large complexes within both the normoxic as well as hypoxic conditions which in the case of the normoxic cultures became a completely confluent monolayer of cells by 4 weeks. However the hypoxic cultures exhibited a striking difference in appearance. Within one month large macroscopic structures formed upon the tissue culture plate surface in stark contrast to the more uniform monolayer appearance typical of the cells cultured under normoxic conditions. The hypoxic cultured cells spontaneously formed a circular structure that measured 2 mm in height and approximately 8 mm in diameter after 2 months in culture (FIG. 1A). The construct was examined after fixation and sectioning and found to demonstrate robust Safranin-O staining, indicative of a proteoglycan-rich matrix (FIG. 1B). During removal using forceps the construct demonstrated viscoelastic properties by resistance to compression and resumption of its' oval shape after forceps compression was released.

After four-weeks in culture cells cultured under normoxia and hypoxia were evaluated using scanning electron microscopy (SEM) and found to have dramatic differences in appearance. After 4-weeks the cells grown under hypoxic conditions had developed large spanning ‘fibrils’ throughout the culture plates whereas the normoxic cultures had simply grown into a confluence of strictly monolayer fibroblastic-appearing cells (FIG. 1 C-D).

### Alginate Globules:

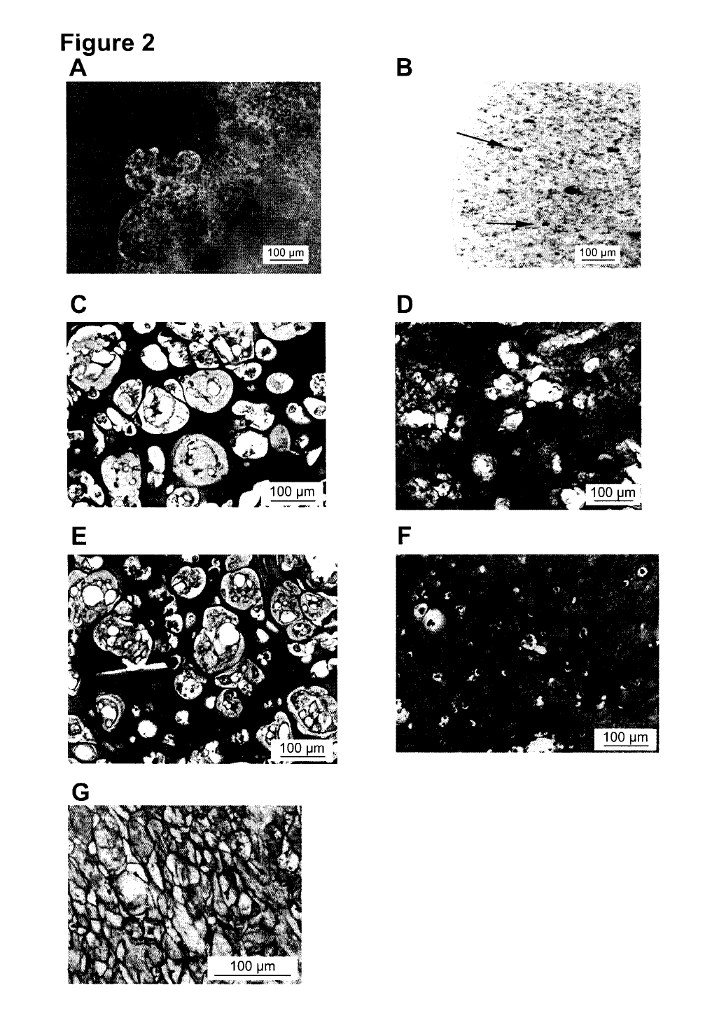

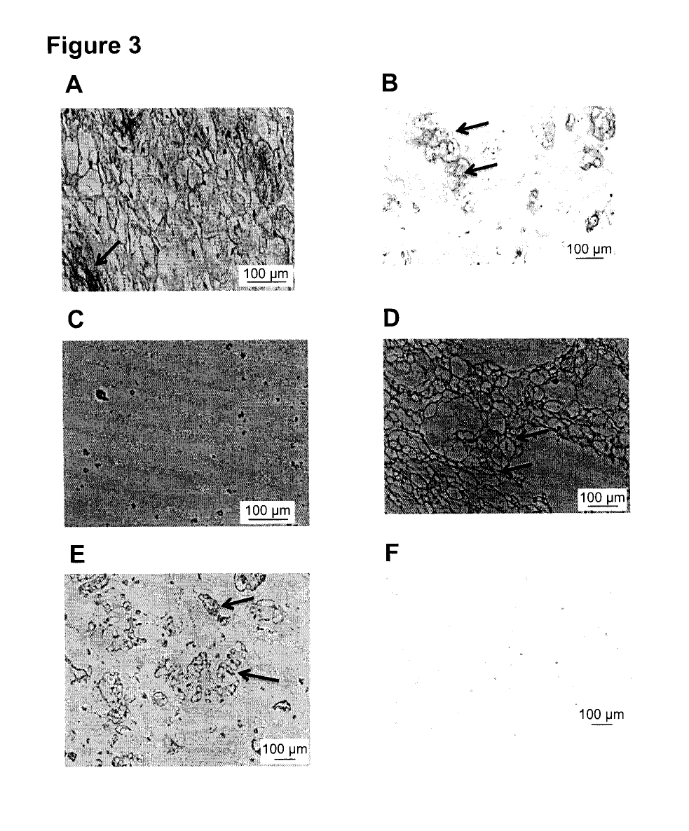

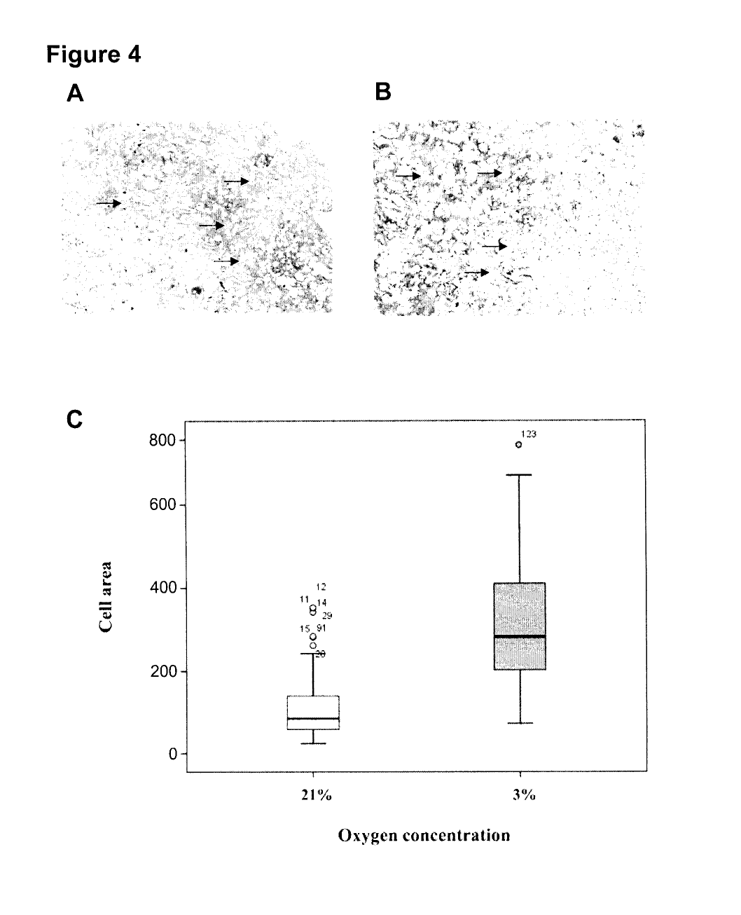

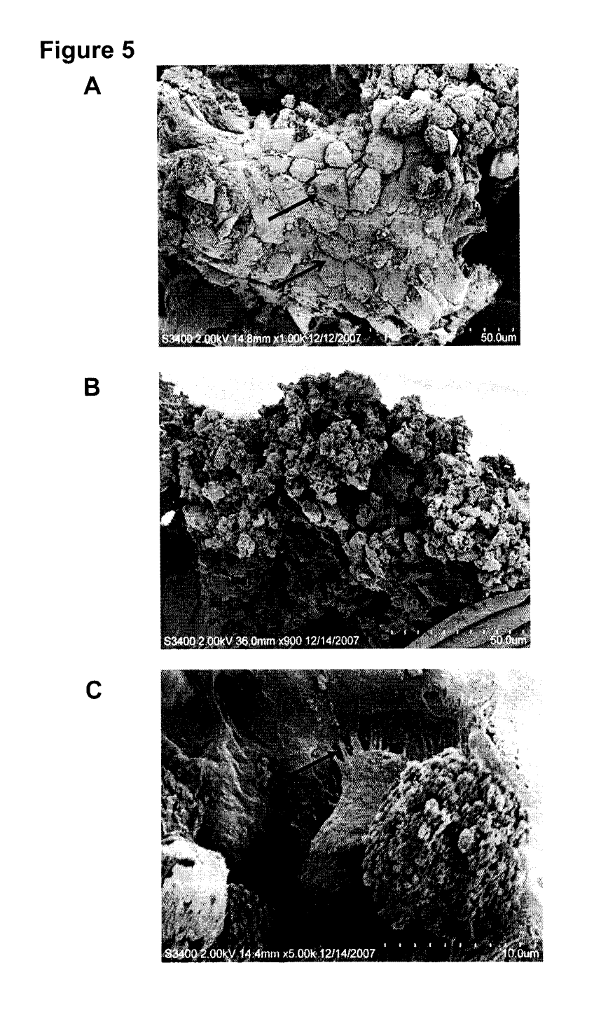

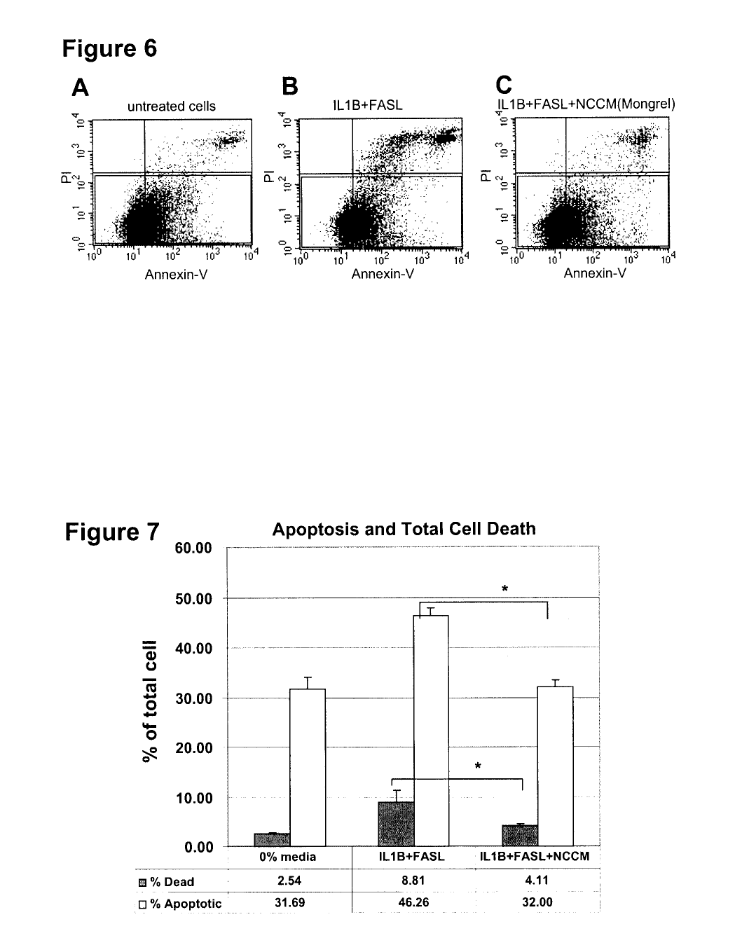

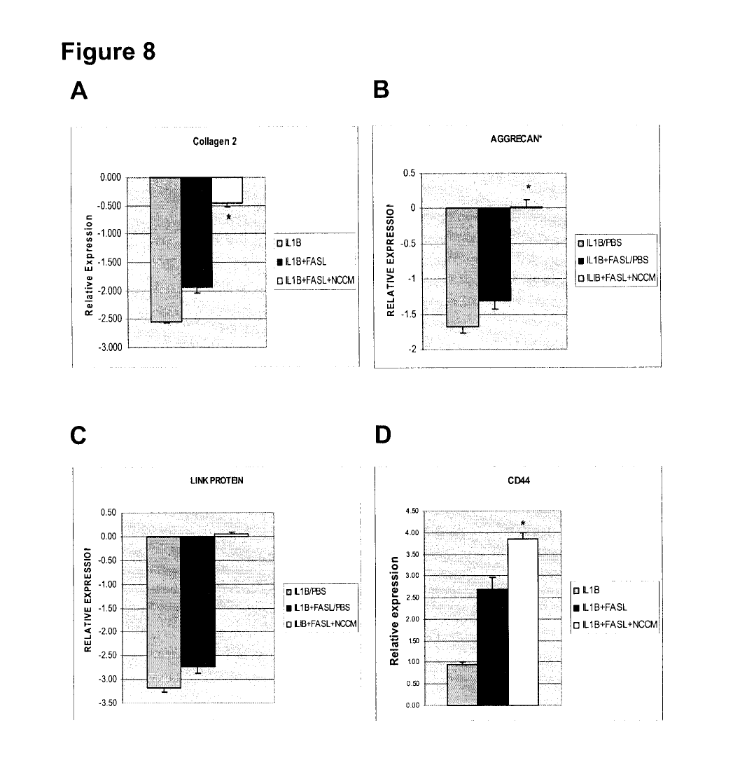

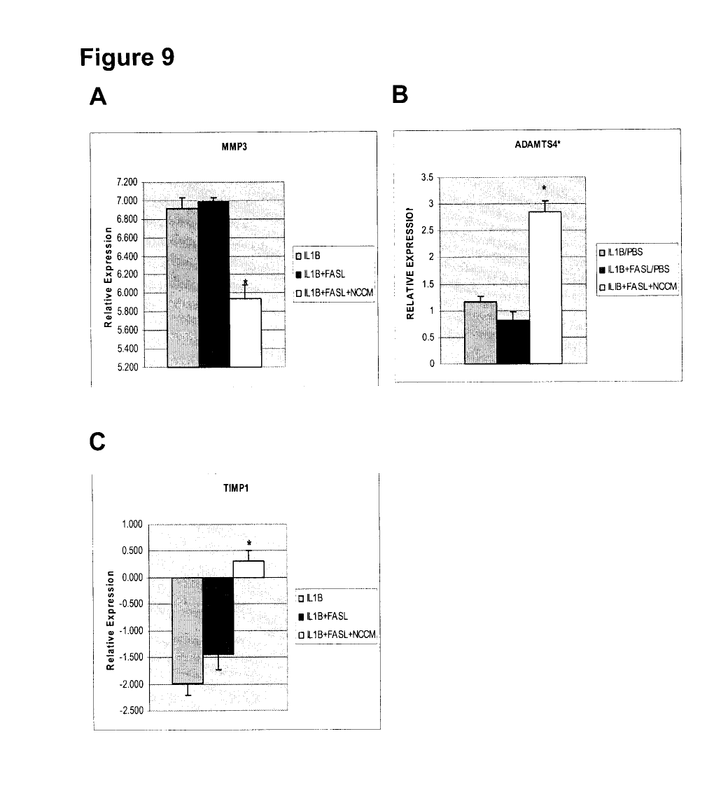

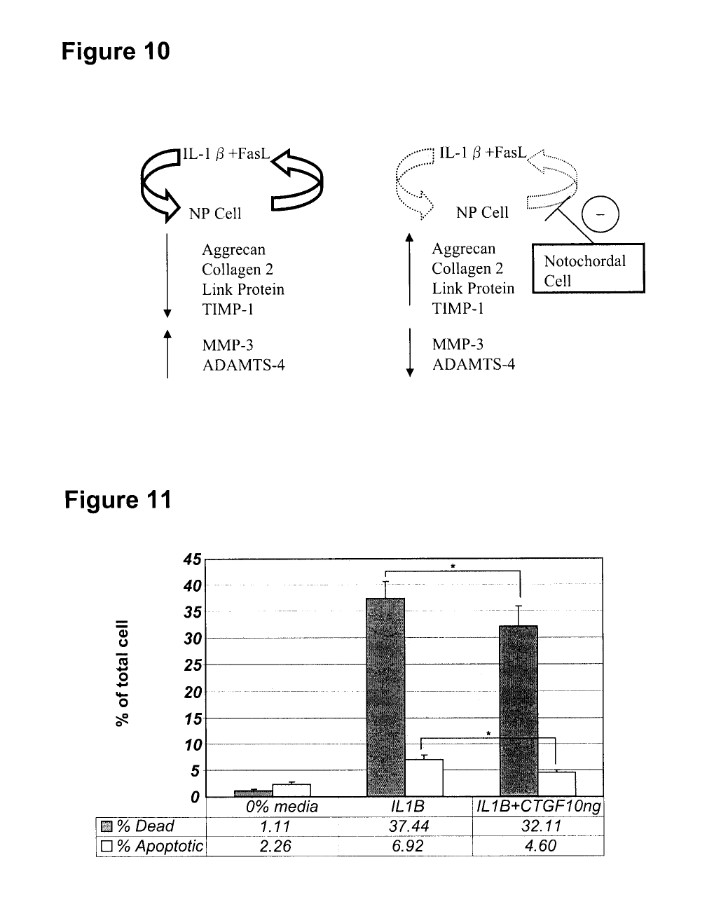

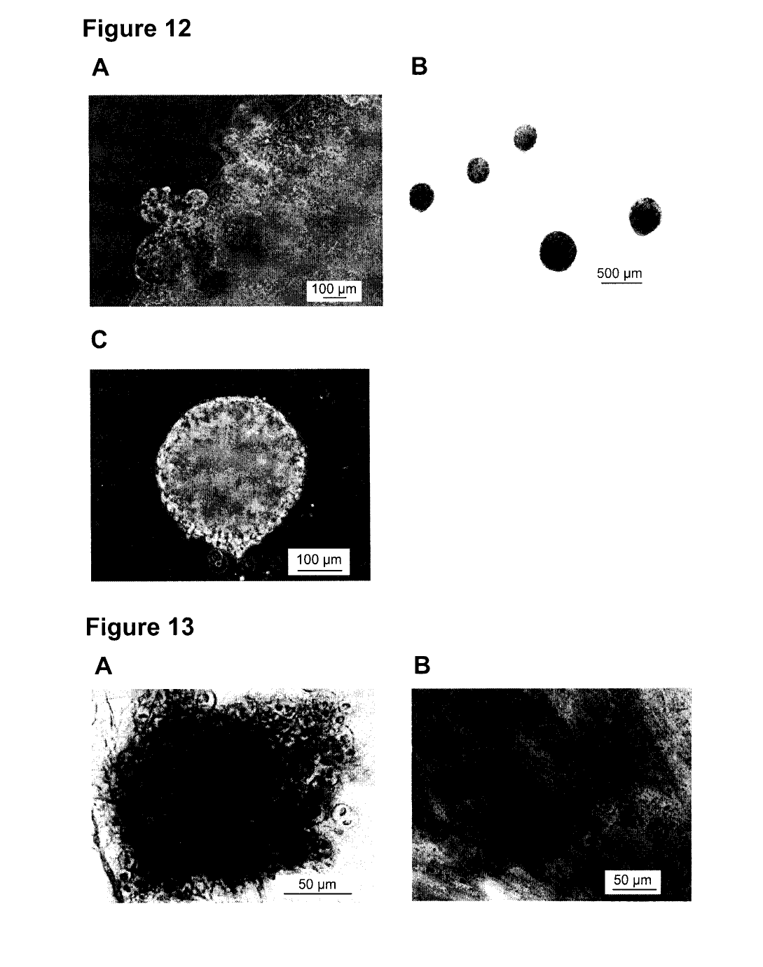

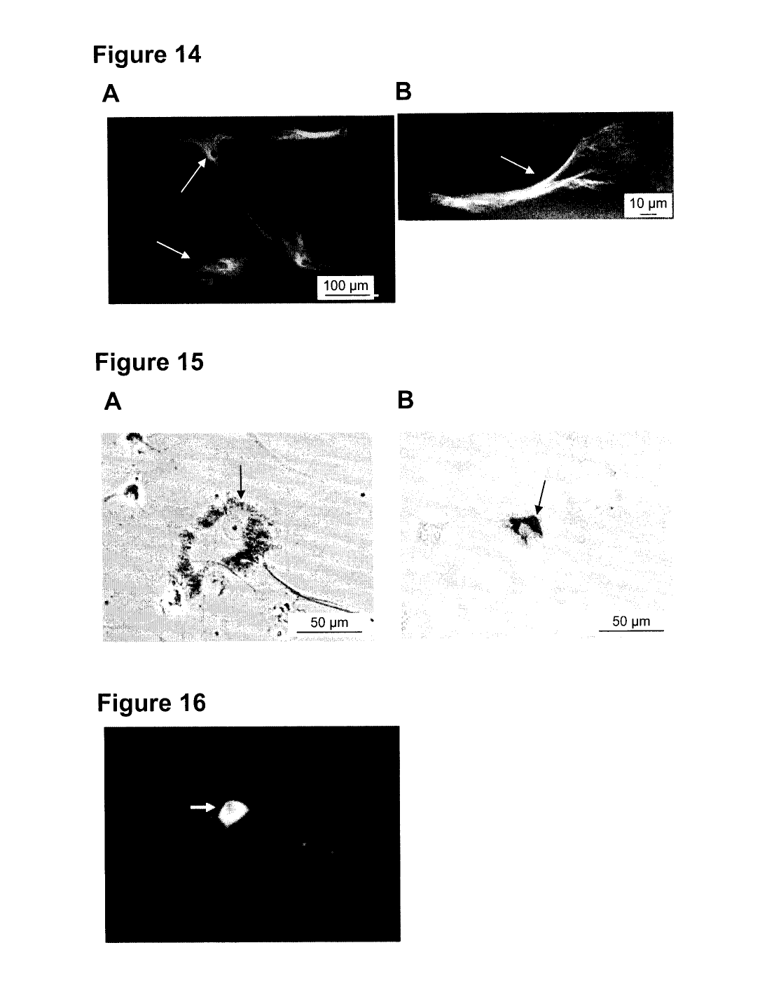

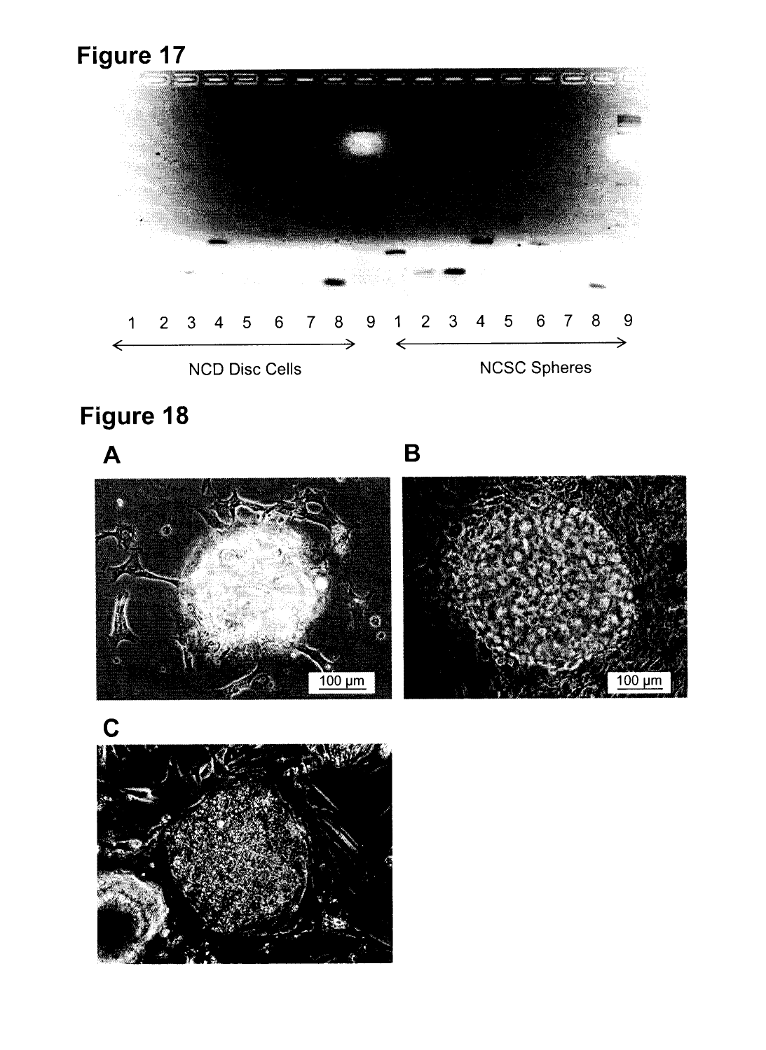

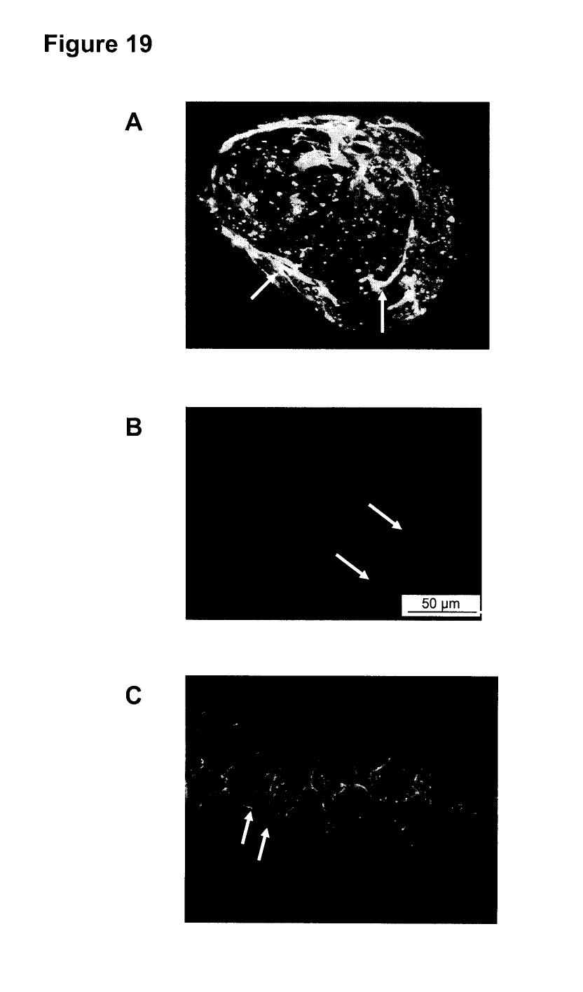

Notochordal cells contained within alginate globules demonstrated marked differences in behaviour under culture in hypoxia as compared to normoxia. The hypoxic cultures formed clusters of cells within two weeks that continued to develop over time. The hypoxic cultures formed dense cell clusters both within the alginate globule as well as many ‘buds’ of cell clusters upon the surface and within the alginate globule (FIG. 2-A). Such behaviour never occurred within globules of cells cultured under normoxia (FIG. 2 B).

### Histological Analysis of Globule Cultures:

The cells cultured under hypoxia demonstrated a robust cellular and extracellular matrix, with large, well defined cells and intense metachromatic staining using Toluidine Blue in stark comparison to normoxic cultures (FIG. 2 C-D). Safranin-O staining was similar confirming the superior histological properties of hypoxic culture as compared to normoxia such that cultures under hypoxia preserved an organized matrix and large cells similar to the in vivo setting and in stark comparison to normoxia (FIG. 2 E-G). The globules containing notochordal cells cultured under hypoxic conditions demonstrated strong immune-reactivity to collagen type II and aggrecan that was very similar in both the in vivo disc NP as well as the hypoxic cultures (FIGS. 3 A-F). However strikingly at 5 months there was no evidence of collagen II and limited, poor aggrecan immune reactivity.

### Histomorphometry:

In general the cells were much larger cultured under hypoxic conditions and showed intact cyto-morphology (intact cell membrane, visible nucleus, and clear cytoplasm-as well as some cells with matrix within the cytoplasm). Normoxic cultures, on the other hand, had far smaller cells, and many did not show the inclusion criteria. In fact, many of the cells did not appear viable as evidenced by a disorganized nucleus, fragmented cell membrane and fragmented/fibrillary appearance on Safranin-O and Toluidine Blue staining and the hypoxic conditions demonstrated significantly larger area than those cultured under normoxia (FIG. 4 A-C). Our morphometric analysis revealed a strikingly statistically significant loss of cell area for cells cultured under normoxic conditions as compared to those under hypoxia.

### Scanning Electron Microscopy:

SEM is an ideal technique with which to observe such structures from a morphological and topographical perspective, therefore in parallel with the histological and immunohistochemical staining, SEM was also performed. Scanning electron microscopy is a high-resolution technique capable of providing clear morphological details of cells and tissues and it is able to show a 3D representation not possible using histological section. The ability of notochordal cells to self-assemble and to assume complex 3D configurations and whether such behaviour occurred in both normoxic and hypoxic conditions was of interest. It was found that a striking difference in cellular behaviour under our two different tissue culture conditions such that the normoxic cultures revealed nothing but fixed and denature alginate whereas the hypoxic cultures formed a robust, complex 3-D tissue ‘construct’ with variable cellular formations including from fibrillary structures, complex cellular networks and the formation of stiffening adhesions between cells of the construct (FIG. 5 A-C).

### Discussion:

### Hypoxia Drives Notochordal Self-Assembly:

Here it was demonstrated for the first time, that notochordal cells thrive under hypoxic tissue culture for at least 5 months and develop an organized, complex 3D extracellular matrix rich in collagen type II and aggrecan; which are vital to the function of the disc nucleus. Parallel notochordal cell cultures harvested from identical sources but cultured under normoxic conditions (21% oxygen) reveal far smaller cells, many of which lack a cell membrane and nucleus and do not appear to survive this length of time in culture. Further, the matrix produced by these cells appears to be remnants of cellular debris and is quite distinct from the hypoxic cultured cells. In addition normoxic cultured cells do not demonstrate either collagen II or aggrecan staining. Hypoxia appears to drive notochordal cells cultured in monolayer to form large fibrillary processes that interconnect cells growing upon the tissue plate surface. This phenomenon was never seen under normoxia. The ability of notochordal cells to ‘self assemble’ under hypoxic tissue culture conditions in both monolayer and 3D culture condition strongly suggests that notochordal cells retain their innate capacity to function as ‘organizer’ as well as structurally supporting cells.

### Developmental Aspects of Notochordal Cells:

During development and morphogenesis cells within the embryo migrate and differentiate along commitment pathways through a complex process termed convergence and extension—such as during gastrulation and neurulation¹². This process of convergence and extension defines how cell populations narrow and lengthen during development and in so doing exert pressure within and around developing cells/tissues of the embryo. Tissues become ‘stiff’ at pivotal times during early development in order for the embryo to properly form. Ordered cellular migration and differentiation is typified by the notochord and its sheath which is one of the earliest areas where tissue stiffness develops in order to provide the longitudinal axis of the developing embryo.

The densely packed cells of the notochord and somatic mesoderm interact with one another and/or with the extracellular matrix between the cells whereby they undergo critical and complex changes in spatial regulation called ‘cellular intercalation’-inclusive of ‘protrusion’ [21]. Protrusions occur in parallel with the development of stiffening adhesions between neighboring cells that allows traction to be imposed upon each other thereby allowing them to produce forceful intercalation and tissue organization²⁰. The ability to resist deformation while at the same time resist re-arrangement is accomplished by virtue of the cells developing in a tightly-knit configuration as well as the development of stiffening adhesions between the cells¹². Stiffening adhesions are thought to develop along the anterior-posterior surfaces of the cell bodies and in so doing resist separation-at the same time they must also have the capacity to assemble and disassemble in concert with growth and re-organization¹². Through the use of scanning electron microscopy it was found that notochordal cells organize into a 3D configuration and over time ‘bud’ within and out of the alginate globule containing the cells as they form a large, interconnected construct. Further, the presence of stiffening adhesions between cells of the tissue construct indicates that under hypoxic conditions, notochordal cells are able to self-assemble a complex 3D cellular and extracellular matrix that in many ways appears to recapitulate key aspects of development (FIG. 5C).

### Cellular and Molecular Aspects of Hypoxia and Nucleus Pulposus Cells:

In order to survive within the hypoxic nucleus, NP cells have been reported to highly express GLUT-1 (glucose transporter protein), a protein that facilitates the entry and utilization of glucose in anaerobic metabolism^{1,16}. Another key factor in the hypoxic environment in many cells/tissues is the multifunctional and atypical growth factor ‘Connective Tissue Growth Factor’ (the current accepted terminology is ‘CCN-2’) which is upregulated by hypoxia through Hif-1-dependent (Hypoxia inducible factor) and TGF-β1-independent pathways¹⁰. CCN-2 expression is regulated by HIF-1a and its expression in many cells is increased under hypoxic conditions¹⁰. It is HIF-1α that regulates such mechanisms as glucose transport, angiogenic factors (where vasculature is present) such as VEGF as well as various genes involved in cell cycle regulation and apoptosis¹⁸. CTGF is a multi-functional molecule known to be involved in a host of cellular processes such as cell proliferation, differentiation, matrix deposition and remodeling, acquisition of tensile strength in cartilage due to its importance in the production of aggrecan and link protein and may have anti- or pro-apoptosis depending upon the cells and tissues involved. With respect to the human condition, it has been reported that HIF-1α is more strongly expressed by nucleus pulposus cells in herniated human discs than normal discs suggesting an attempt at recovery/repair-however there is no explanation for the mechanism at play under these circumstances [31, 32].

It has been reported that rat nucleus pulposus cells increase their expression of the classic chondrogenic genes collagen II, aggrecan and Sox9-genes when cultured for 36 hours under hypoxia providing further evidence of the beneficial effect of hypoxia¹⁷. In this study it was observed that hypoxia activates Akt signaling, a factor considered important in the maintenance of Glut-1 transcriptional activity and therefore anaerobic delivery of glucose to the cells. Further, activation of MEK1/ERK pathways by hypoxia was reported to confer protection from apoptosis in NP cells induced by hypoxia and the products of the activation of these pathways may be one mechanism whereby NP cells adapt to their environment¹⁷.

Notochordal Cells Cultured Under Hypoxic Conditions Produce a Matrix Indistinguishable from the In Vivo Setting:

It has been demonstrated that notochordal cells of the NCD canine secrete a form of aggrecan that is superior to that formed by the CD notochordal cells in that the aggrecan formed by NCD notochordal cells assembles into aggregates further away from the cell surface and in so doing allows for a superior hydrophilic capacity⁶. Owing to the resistance to DDD and greater biomechanical load-bearing capacity of the NCD IVD that it has been postulated that the IVD that is rich in notochordal cells may confer superior biomechanical properties as compared to the more fibrocartilagenous disc¹⁴.

The results of this study suggests that future strategies focused upon cell-based therapies particularly involving notochordal cells should strive to culture such cells under hypoxic conditions since normoxia over the long term does not support cell survival and matrix deposition. Further, hypoxia seems to provide the single necessary component to activate notochordal cells to proliferate and produce an abundant, biologically relevant extracellular matrix and to undergo self-assembly-akin to that very process which occurs during development. Understanding the biological mechanisms underlying the robust responses of notochordal cells to hypoxic tissue culture, the capacity of notochordal cells to stimulate other NP cells and particularly the role(s) played by reactive oxygen species will allow for future mechanistic investigations of the responses of these cells to low oxygen environments. It may be that notochordal cells act as a kind of regenerative reservoir capable under the right conditions of inducing restorative processes that could potentially be harnessed in a future cell-based therapy for the treatment of degenerative disc disease.

### REFERENCES FOR BACKGROUND AND EXAMPLE 1

1. Agrawal A, Guttapalli A, Narayan S, Albert T J, Shapiro I M, Risbud M V: Normoxic stabilization of HIF-1alpha drives glycolytic metabolism and regulates aggrecan gene expression in nucleus pulposus cells of the rat intervertebral disk. American Journal of Physiology-Cell Physiology August; 293 C621-631, 2007
2. Aguiar D J, Johnson S L, Oegema Jr T R: Notochordal cells interact with nucleus pulposus cells: regulation of proteoglycan synthesis. Experimental Cell Research 246:129-137, 1999
3. Arnott J A, Nuglozeh E, Rico M C, Arango-Hisijara I, Odgren P R, Safadi F F, et al: Connective tissue growth factor (CTGF/CCN2) is a downstream mediator for TGF-beta1-induced extracellular matrix production in osteoblasts. Journal of Cell Physiology March; 210:843-852, 2007
4. Barrionuevo F, Taketo Makoto M, Scherer G, Andreas K: Sox9 is required for notochord maintenance in mice. Developmental Biology 295:128-140, 2006
5. Behrens A, Haigh J, Mechta-Grigoriou F, Nagy A, Yaniv M, Wagner E F: Impaired intervertebral disc formation in the absence of Jun. Development January; 130(1):103-109 2003
6. Cappello R, Bird J L, Pfeiffer D, Bayliss M T, Dudhia J: Notochordal cell produce and assemble extracellular matrix in a distinct manner, which may be responsible for the maintenance of a healthy nucleus pulposus. Spine April 15-31:873-882, 2006
7. Erwin W M, Ashman K, O'Donnell P, Inman R D: Nucleus pulposus notochord cells secrete connective tissue growth factor and up-regulate proteoglycan expression by intervertebral disc chondrocytes. Arthritis and Rheumatism December 54:3859-3867, 2006
8. Erwin W M, Inman R D: Notochord cells regulate intervertebral disc chondrocyte proteoglycan production and cell proliferation. Spine May 1:1094-1099, 2006
9. Goetzel R A, Hawkins K, Ozminkowski R J, Wang S: The health and productivity cost burden of the “top 10” physical and mental conditions affecting six large U.S. employers in 1999. Journal of Occupational and Environmental Medicine 45:5-14, 2003
10. Higgins D F, Mangatt P G, Yasuhiro A, Wutz A, Johnson S R, Haase V H: Hypoxic induction of CTGF is directly mediated by Hif-1. American Journal of Physiology-Renal Physiology 297:F1223-F1232, 2004
11. Holm S M A, Urban J, et al: Nutrition of the intervertebral disc. Solute transport and metabolism. Connective Tissue Research 8:101-119, 1981
12. Keller R, Davidson L, Edlund A, Elul T, Ezin M, Shook D, et al: Mechanisms of convergence and extension by cell intercalation. Philosophical Transactions of the Royal Society-Biological Sciences 355:897-922, 2000
13. Kuroki K C, Cook J L, Kreeger J M, Tomlinson J L: The effects of TIMP-1 and -2 on canine chondrocytes cultured in three-dimensional agarose culture system. Osteoarthritis and Cartilage 11:625-635, 2003
14. Oegema Jr. TR: The role of disc cell heterogeneity in determining disc biochemistry: a speculation Biochemical Society Transactions 30:839-844, 2002
15. Paul R, Haydon R C, Cheng H, Ishikawa A, Nenadovic N, Jiang W, et al: Potential use of Sox9 gene therapy for intervertebral degenerative disc disease. Spine 28:755-763, 2003
16. Rajpurohit R, Risbud M V, Ducheyne P, Vresilovic E J, Shapiro I M: Phenotypic characteristics of the nucleus pulposus: expression of hypoxia inducing factor-1, glucose transporter-1 and MMP-2. Cell and Tissue Research June; 308:401-407. Epub 2002 May 2025, 2002
17. Risbud M V, Fertala J, Vresilovic E J, Albert T J, IM S: Nucleus pulposus cells upregulate PI3K/Akt and MEK/ERK signaling pathways under hypoxic conditions and resist apoptosis induced by serum withdrawl. Spine 30:882-889, 2005
18. Schipani E, Ryan H E, Didrickson S, Kobayashi T, Knight M, Johnson R S: HIF-1a is essential for chondrocyte growth arrest and survival. Genes Development 15:2865-2876, 2001
19. Selard E, Shirazi-AdI A, Urban J P: Finite element study of nutrient diffusion in the human intervertebral disc. Spine September 1; 28 1945-1953, 2003
20. Shih J, Keller R: Cell motility driving mediolateral intercalation in explants of Xenopus laevis. Development 116:915-930, 1992
21. Spangenberg K M P G, Trahan C A, Randolph M A, Bonassar L J.: Histomorphic analysis of a cell-based model of cartilage repair. Tissue engineering in orthopaedic surgery 8:839-846, 2002
22. Takegami Kenji, Thonar Eugene J, An Howard S, Kamada Hiroshi, Masuda K M: Osteogenic Protein-1 Enhances Matrix Replenishment by Intervertebral Disc Cells Previously Exposed to Interleukin-1. Spine 27:1318-1325, 2002
23. Thompson J P, Oegema Jr. T R, Bradford D S: Stimulation of mature canine intervertebral disc by growth factors. Spine 16:253-260, 1991
24. Tim Yoon S, Su Kim K, Li J, J S P, Akamaru T, Elmer W A, et al: The effect of bone morphogenetic protein-2 on rat intervertebral disc cells in vitro. Spine 28:1773-1780, 2003
25. Urban J P: The role of the physicochemical environment in determining disc cell behaviour. Biochem Society Transactions November; 30 (Pt 6):858-864, 2002
26. Urban J P, Smith S, Fairbank J C: Nutrition of the intervertebral disc. Spine December 1; 29:2700-2709, 2004
27. Vadala G, Studer R K, Sowa G, Spiezia F, Lucu C, Denaro V, et al: Coculture of bone marrow mesenchymal stem cells and nucleus pulposus cells modulate gene expression profile without cell fusion. Spine 33:870-876, 2008
28. Yoon S T, Park J S, Kim K S, Li J, Attallah-Wasif E S, Hutton W C, et al: LMP-1 upregulates intervertebral disc cell production of proteoglycans and BMPs in vitro and in vivo. Spine 29:2603-2611, 2004
29. Zhang Y, Anderson D G, Phillips F M, Thonar E J-M A, He Tong-Chuan, Pietryla D, et al: Comparative effects of bone morphogenetic proteins and sox9 overexpression on matrix accumulation by bovine anulus fibrosus cells. Spine 32:2515-2520, 2007

### Example 2

### Hypoxic Conditions Induces Expansion of Multi-Potency Stem Cells from Non-Chondrodystrophic Canine Notochordal Cells

### Introduction:

The notochord is a critical organizing structure or mesodermal origin formed very early in embryogenesis. The notochord begins as a primitive streak and ultimately matures to form the notochord which defines the longitudinal axis of the embryo, provides structural integrity to the notochord as well as ‘stiffness’. In addition, the notochord is a source of critical differentiation signals to surrounding undifferentiated cells whereby neural crest cells migrate dorsally to form the neural tube and ultimately the spinal cord. In addition, the mesenchymal tissues condense to form the sclerotome which ultimately become cartilage, bone, and ligament and musculature of the spine including the vertebrae. The notochord itself eventually becomes segmented and compartmentalized as the nucleus pulposus of the intervertebral disc.

Persistence of notochordal cells (NC cells) within the intervertebral disc (IVD) nucleus pulposus (NP) has been equated with ‘protection’ from the development of degenerative disc disease (DDD)-a condition marked by gradual and progressive loss of function of the disc that may bring with it chronic, debilitating spinal and radicular pain. DDD occurs as a loss of function of the intervertebral disc as the degenerative cascade continues and the motion segment made of the two adjacent vertebrae and the intervening IVD becomes compromised. Such compromise results in a loss of disc height, hypertrophy of the posterior joints, bulging and/or herniation of the IVD NP and impingement of spinal nerves and/or the spinal cord. Indeed, spinal cord impingement particularly in the cervical spine may result in an extremely morbid condition known as cervical spondylotic myelopathy (CSM), a condition that may lead to chronic disability and tetraparesis.

The unique sub-species of canine, the non-chondrodystrophic (NCD) canine is protected from developing DDD whereas the chondrodystrophic canine (CD) suffers early and significant DDD in a strikingly similar fashion as does the human. Several reports have been published concerning the ability of notochordal cells obtained from NCD canine NC cells to induce NP cells to increase proteoglycan production, increase NP cell proliferation and some of the proteins produced by NCD NC cells-including CCN-2 has been characterized. It has been further recently reported that hypoxic culture of these NCD NC cells results in the development of a robust tissue construct that is strikingly similar to the IVD NP of the NCD canine. Here it is demonstrated that what appears to be a sub-population of these NCD NC cells when cultured under hypoxia even in totally serum-deficient conditions can thrive for over 7 months and de-differentiate into a pluripotential phenotype. Further, our novel hypoxic tissue culture system allows the NCD NC cell when cultured under hypoxia to de-differentiate to naturally express neuroprogenitor cells-thus reconstituting the milieu of embryological development. This novel and unique approach has for the first time demonstrated that NCD NC cells retain the biological capacity to de-differentiate into pluripotential cells-a phenomenon that may be harnessed within a regenerative approach to the treatment of a plethora of spine-related disease involving DDD and neural repair.

### Methods:

8-10-month non-chondrodystrophic canines were obtained after failing at adoption and the IVD NP cells were obtained according to our established protocols. The cells were then placed into 1.2% alginate ‘globule’ culture using Advanced DMEM/F-12 medium supplemented with penicillin, streptomycin and fungizone 100 U/mL (PSF), L-glutamine and variable fetal calf serum (FCS) ranging from totally serum-free to 8% FCS in 2% steps (2, 4, 6 and 8%). The cells were maintained in culture within 3.5% oxygen, 5% CO₂ and 95% humidity in a Nuaire™ variable gas incubator. 50% of the medium was changed each 3 days and the cultures continue to be viable for over 7 months.

The cultures were observed weekly and when ‘spheres’ of cells and complex cellular constructs appeared they were examined in a number of methods.

1. Frozen section: Globules containing the spheres and complexes were fixed in a 2-step process involving 4% Paraformaldehyde in 0.1M Cacodylate buffer containing 10 mM CaCl₂ (pH 7.4) for 8 hours at room temperature followed by overnight rinse in 0.01M Cacodylate buffer containing 50 mM BaCl₂ (pH 7.4). Next the globules were cryoprotected in 30% sucrose, embedded in OTC frozen and sectioned at 20 μm using a cryostat (−20° C.), mounted on glass slides and stained for immunofluorescence.
2. Recovery from Globules: Other globules were dissolved using 55 mM Sodium Citrate in 150 mM NaCl, the cells were washed three times with PBS (pH 7.4) and finally re-suspended in complete Advanced DMEM/F-12 medium on round cover slips coated with Matrigel™ within 4-well mini-plates and cultured under hypoxia.
3. Recovery and Density Gradient Separation: After dissolving with sodium citrate, the cells were recovered as in (2) above and then subjected to Percoll density gradient separation. The gradient was developed in 1 mL volume at 1.007, 1,014, 1.021, 1.028 and 1.035 specific gravity steps and the cell mixture was layered on top and centrifuged with no brake at 200×G for 20 minutes. The cells were recovered from each layer and the spheres and complexes were by far more numerous within the 1.014 and 1.021 levels. These cells were then re-cast within 1.2% sodium alginate globules in 0.5 mL volumes and placed back within hypoxic culture. These samples were cultured for an additional 6 weeks where it was found that an extremely dense population of spheres and constructs developed from the initial seeding.

Immunofluorescence: Appropriate samples were examined using fluorescence microscopy and stained for the presence of: Beta III tubulin, Nestin, GFAP, NCAM, Sox2 and Oct3/4. These proteins are established markers of neural progenitor lineage and in the case of Sox2 and Oct 3/4, of pluri-potential capacity. Representative sections of NCD canine IVD NP tissues as controls have been examined. Negative controls lacked the primary anti-body.

### Results:

It is previously reported that hypoxic culture of NCD canine NC cells produce complex 3D tissue constructs that bare a striking similarity to the histological appearance of the NP of the NCD canine-an animal naturally protected from the development of DDD. Scanning electron microscopic imaging of these constructs has been performed and it is found that in addition to the complex cellular and extracellular matrix formed by these cells, they also develop stiffening adhesions between the cells providing evidence of cellular intercalation and extension-type of development-typical of that seen in embryological development.

It has also been demonstrated that that these same NCD NC cells have a sub-population of cells that are capable of forming ‘spheres’ that bear typical resemblance to stem cell spheres (FIG. 12 A). The spheres are multi-cellular and when they attach to the tissue culture plate they differentiate but the sphere appearance is maintained for a period of weeks or longer upon a monolayer of cells that appear to function as ‘feeder’ cells (FIG. 18B). NCD NC cells when cultured under hypoxia reliably form these spheres across many different samples of animal and under all tissue culture medium conditions and interestingly-even under conditions that are totally serum-free. Advanced DMEM/F-12 supplemented with antibiotics and L-glutamine has been used as described earlier and from totally serum-free up to 8% FCS supplementation. Under serum-free conditions although the development of the spheres is slower, such development does reliably occur in repeated experiments.

The appearance of the notochordal spheres is of striking resemblance to neurospheres formed by in vitro culture of stem cells obtained from mammalian central nervous system located in areas such as the periventricular area of the forebrain lateral ventricles (The EMBO Journal (2005) 24, 3504-3515). These pluripotential cells reside within niches in which complex control mechanisms and microenvironments regulate the metabolism and expression of these cells where the cells may be called upon in the case of injury/disease to differentiate in order to replace damaged/injured cells. In the normal circumstance however these cells remain quiescent within their niche-tightly controlled by the local environment. In the adult situation progenitor cells act as a reservoir for repair where specialized cells may be repaired as well as provide a source of replenishment of specialized cells and normal turnover of organs with regenerative capacity such as skin and intestinal tissues. Adult stem cells are similar to embryonic stem cells in that they have the ability to differentiate into multiple cell types-however in a departure from the embryonic stem cell, adult stem cells are often restricted to their specific lineage. “Transdifferentiation” is the process whereby a stem cell of one lineage is able to differentiate into a different lineage-and adult stem cells vary in their ability to transdifferentiate (The EMBO Journal (2005)24, 3504-3415).

Immunhistochemical analysis has demonstrated that hypoxia induces NCD NC cells to express Beta-III tubulin (FIG. 19A), GFAP (FIG. 14B), and NCAM (FIG. 19C)-all of which are well know neuroprogenitor markers. In addition, preliminary data exists indicating that the cells contained within the spheres express Sox2, a well known marker of pluripotential stem cells (FIG. 19 B)

Beta-III Tubulin is a Microtubule Element within the Tubulin Family of Globular proteins found almost exclusively in developing neurons. During development, class III β-tubulin is thought to be one of the earliers neuron-specific cytoskeletal markers. The expression of Beta-III tubulin is considered to classify the tissue in which it is detected to be of an early commitment to neuronal cell lineage (Dennis K, Uttenbogaard M, Chiaramello A. Moody S A: Cloning and characterization of the 5′-flanking region of the rat neuron-specific class III β-tubulin gene. Gene 2002; 294:269-277).

Nestin expression is usually of a transient nature during developmental phases and is expressed by many types of cells. However, nestin expression is usually transient and is not known to persist into adulthood. Nestin is an intermediate filament protein expressed in dividing cells during the early stages of development in the CNS, PNS and in myogenic and other tissues. Once cells differentiate, nestin is downregulated and is replaced by tissue-specific intermediate filament proteins. Nestin expression is known to persist in adulthood in the case of neuronal precursor cells of the subventricular zone within the brain. During neuro- and gliogenesis, nestin is replaced by cell type-specific intermediate filaments, such as neurofilaments and glial fibrillary acidic protein (GFAP). In situations such as pathological scarring in CNS injury nestin expression is re-induced within formation of the glial scar and during regeneration of injured muscle tissue.

In addition to forming complex 3D tissue constructs, the hypoxic culture of notochordal cells in vitro appears to activate what appears to be a sub-population of these cells. These notochordal cells are harvested from the intervertebral disc nucleus pulposus of skeletally mature non-chondrodystrophic canines-an environment that is an ischemic compartment completely isolated from the rest of the body and is totally devoid of innervation. Some of the cells harvested from this tissue compartment appear to either de-differentiate or ‘transdifferentiate’ (in this case demonstrating a neural lineaage phenotype) when cultured under hypoxic conditions or these pluripotential stem cells may represent a hitherto unknown regenerative reservoir within the disc without the use of specific neurotrophic media. This differentiation suggests that there may be a reservoir of pluripotential cells within the disc nucleus pulposus of these animals that may revert to a more pluripotential phenotype under the influence of hypoxia or these cells already present may become activated under these conditions.

The human IVD nucleus pulposus forms the same way as other mammals-under considerable influence of the notochord and signals secreted by the notochord. Notochordal cells obtained from an animal protected from developing degenerative disease when cultured under biologically relevant conditions form complex, organized nucleus pulposus tissue constructs that are strikingly similar to that seen in vivo. Further, nucleus pulposus cells obtained from these animals appear to contain a sub-population of cells that when cultured over time transdifferentiate and express proteins found in pluripotential cells.

Notochordal cells secrete soluble factors that act upon local undifferentiated mesenchymal cells in development which in turn terminally differentiate into mature cells such as cartilage, neurons, bone and muscle. Our observations indicate that notochordal cells are capable of self-renewal and under totally serum-free conditions are viable to at least 7 months. Such cells that continue to thrive now into their 11^{th} month of hypoxic in vitro culture currently exist. They continue to form ‘spheres’ and large, complex tissue constructs within alginate ‘globules’. It is anticipated that notochordal cells and/or their secreted products could be exploited within the context of regenerative medicine applications-in particular for the treatment of disc disease. Applications of our novel technology-hypoxic culture of notochordal cells could be used in the following manner:

1. Soluble factors produced by notochordal cells may be used to culture adult bone marrow-derived stem cells to assume a ‘nucleus pulposus’ phenotype. Suitably ‘transformed’ stem cells could then be re-implanted within suitable patients either intra-operatively or through percutaneous means. These cells will by definition have assumed the characteristic phenotype necessary to survive within a hypoxic environment.
2. It may be that the sub-population of notochordal cells that form ‘spheres’ retain the unique ability to act as repair and maintenance cells. The ability of these cells to form such spheres and their ability to display characteristics of progenitor cells is herein demonstrated. Therefore these cells could be harvested and co-cultured with bone marrow-derived cells in order to induce the stem cells to assume the nucleus pulposus progenitor phenotype characteristic of the notochordal progenitors.
3. A novel in vitro method of culturing disc-derived cells that is able to sustain cells to date for almost one year has been developedand the cells continue to thrive. It is found that unlike normoxic tissue culture conditions in which the cells die, under hypoxia the cells thrive. The intention is to design a pressurized, hypoxic tissue system that will even more closely mimic in vivo conditions. Such a pressurized, hypoxic tissue culture system would reflect a novel and unique methodology with which to engineer disc cells for regenerative applications.

### Example 3

### Hypoxia Confers Pro-Survival Characteristics Upon Notochordal Cells and the Generation of Complex Tissue Constructs In Vitro

### Introduction:

Degenerative disc disease (DDD) is an extremely common and expensive healthcare ailment which unlike advances in biologic therapies for fracture management and disease modifying drugs for various arthropathies has no curative strategy which attenuates or reverses the degenerative cascade [1]. With respect to the pursuit of a biological intervention the study of the non-chondrodystrophic canine (NCD canine) is noteworthy in that this animal is unique amongst the canine sub-species in that these dogs preserve their notochordal cell populations and are protected from the development of degenerative disc disease (DDD) [2, 3]. It has been previously determined that non-chondrodystrophic canine intervertebral discs-derived notochordal cells secrete Connective Tissue Growth Factor (CTGF/CCN-2) and that conditioned medium obtained from these cells up-regulates important matrix gene expression, cell proliferation and proteoglycan production in nucleus pulposus (NP) cells [4, 5]. CTGF is known to be an important regulatory molecule in development that works in conjunction with a number of transcription factors and other developmental genes such as jun, sox5,6,9, hedgehog, and noggin, important genes in disc formation and chondrogenesis [6, 7].

The intervertebral disc is a highly avascular compartment that is almost totally devoid of vascular supply in the mature adult. Therefore nutrient balance in the intervertebral disc (IVD) is likely tightly regulated by both the cells within and structural aspects of the disc including the vertebral endplates. The importance of nutrient and gas diffusion to the disc (a structure otherwise devoid of vasculature) has been highlighted by a number of studies [8-11]. It has been reported that the capillary networks present at the vertebral endplates are up to four times more dense directly over the disc nucleus as compared to the annulus—suggestive of the vital supply of such nutrients to disc cells in the absence of direct vascular supply [9, 10]. Nucleus pulposus cells (NPC) have necessarily adapted to survive within a hypoxic and relatively nutrient-poor environment, the oxygen concentration within this compartment reportedly being between 2-5% [9]. However most studies concerning intervertebral disc metabolism within the in vitro setting have studied these cells cultured under classical tissue culture conditions of 21% O_{2 [}3-5, 12-16]. A number of reports concerning the delivery of some of these factors have claimed effective restoration of disc height in vivo and/or increased expression of desirable genes such as aggrecan and collagen. However the tissue culture periods used are often only a matter of days and the cultures are also often maintained within monolayer. Further, nucleus pulposus cells assume a fibroblastic phenotype in monolayer culture which is very different from that found in the in vivo setting [16-18].

In order to develop effective future biological therapeutics that may involve gene transfer, the injection of growth factors and/or stem-cell based therapy it is vital that such interventions be designed and studied in a biologically relevant system. Therefore it is critical to determine the tissue culture conditions under which such critical applications must be performed and to our knowledge have never before been examined in detail, particularly with respect to notochordal cells which are vital to the developmental organization of the disc nucleus.

The realities of the hypoxic, ischemic environment within the disc nucleus create a unique challenge to the investigator to draw conclusions from in vitro experimentation that is biologically credible and biologically relevant. There are a number of potential biologically-based therapies currently under development and reported in the literature [15, 16, 19]. However the studies that reflect in vitro experimentation have been largely performed under classical tissue culture oxygen saturation, a condition that does not mirror the in vivo condition. Furthermore, the development of a biologic therapy to treat DDD requires that preliminary studies must be performed within the context of biologically relevant experimental approaches with an emphasis upon primary cells. Primary cells reflect biological realities that may not be the case when using cell lines obtained by immortalization. The biology of primary disc-derived notochordal cells is of particular interest since it is these cells that provide an anabolic/matrix protective function upon NP cells [4, 5]. Therefore, in this study the ability of primary notochordal cells to survive under long-term culture was chosen for evaluation in order to examine the quality of the matrix that they produce and to compare these variables with traditional normoxic tissue culture.

The healthy disc nucleus maintains a range of O₂ concentration of between 2-5%; therefore for hypoxic studies a mid-range value of 3.5% was chosen. During pilot studies it was found that maintaining 2% O₂ levels, though certainly possible, required an inordinate usage of nitrogen gas in order to displace the O₂. Hypoxic responsive genes are active below 5% O₂ therefore 3.5% satisfied both the requirements of hypoxic culture and did not consume nitrogen at a prodigious rate.

Here for the first time it is reported that primary notochordal cells cultured under hypoxic conditions thrive and form complex 3D tissue and remain viable for at least 5 months.

### Methods and Materials:

### Non-Chondrodystrophic Canine Disc Notochordal Cell Cultures

Notochordal cells were obtained by meticulous dissection of the nucleus pulposus from five freshly sacrificed non-chondrodystrophic canine lumbar spines (unclaimed animals that had failed at adoption). Within 2 hours of euthanasia the lumbar spines were removed, the spinal soft tissues dissected away and the spines were washed with DH₂0, Clidox™, and then liberally rinsed with Betadine™ and cooled to 4° C. The discs were placed into a 70 mm² Petri dish and suspended within Advanced DMEM/F-12 supplemented with penicillin, streptomycin and fungizone (PSF) and 5% fetal calf serum (FCS) and placed into 3.5% O₂ and 5% CO₂. The following day the nuclei pulposi were meticulously separated from any remaining annulus and washed three times with medium. Next the isolated nucleus pulposus tissues were sequentially enzymatically digested according to our pre-established methods [5]. The following day the cells were rinsed, filtered through 70 μm cell strainers (Falcon) and examined microscopically. Notochordal cells obtained in this fashion produced a virtually pure population of notochordal cells and density gradient separation techniques were not required. The cells were then counted and washed three times with medium and then centrifuged at 500×g. The cell pellet was then mixed with 1.2% sodium alginate at a concentration of 3×10⁶ cells/mL. 0.75 mL of the alginate/cell suspension was pipetted into each well of a 6-well plate containing 102 mM CaCl₂ to form an oval alginate ‘globule’. The globule was allowed to polymerize for 15-20 minutes and was then rinsed three times with PBS (pH 7.4) and finally replaced with complete DMEM/F-12 advanced medium supplemented with 8% FCS and PSF (100 U/mL Penicillin, streptomycin, fungizone) and the medium replaced each three days throughout the culture period. Other notochordal cells were removed and plated directly into 6-well plates at a density of 0.5×10⁶ cells/well and complete medium added to the wells. The ‘globule’ and monolayer cultures were then inserted into either a standard incubator set to 3° C., 5% CO₂ and 21% O₂ or a hypoxic incubator set to 3° C., 5% CO₂ and 3.5% O₂ (Nuaire) and were monitored each two days and followed over selected time periods for up to 5 months under both normoxia and hypoxia. The cells cultured under hypoxic conditions were maintained under hypoxia during feeding by transferring the cultures to a glove box set to the identical conditions of hypoxia (Coy). The cultures were photographed each two weeks as they were observed over time.

In order to compare ‘native’ intervertebral disc nucleus pulposus and the hypoxic and normoxic cultured cells IVD's were obtained from the same animals that were used for the source of notochordal cells in all other experiments by removing the underlying vertebrae complete with the attached IVD. Therefore the specimens were harvested directly from dissection fixed them in 4% paraformaldehyde and decalcified in EDTA for one week and then embedded the specimens in paraffin. These specimens were then sectioned at 4 μm, mounted on cover slips and stained using Safranin-O for histomorphometric analysis (see below for details) and for immune-like reactivity to anti-collagen type II and aggrecan.

### Histological Analysis of Notochordal Cells:

At the appropriate time points some of the alginate globules were removed, fixed and embedded in paraffin, sectioned and stained. Briefly, fixation of the globules consisted of using 0.1 M Cacodylate (Sigma) in 10 mM CaCl₂ and 4% paraformaldeyde pH 7.4. The globules were left a room temperature for 8 hours and then switched to 0.1 M Cacodylate buffer containing 50 mM BaCl₂ pH 7.4 and changed 4 times over 16 hours and kept at 4° C. The globules were then removed from buffer and embedded in paraffin sectioned, dehydrated through alcohols and xylenes stained and cover-slipped. Toluidine Blue, Safranin-O, were used to detect for immune-like reactivity to collagen type II and aggrecan. Five months of culture was chosen as the endpoint in order to ascertain the behaviour of the cultures over long-term conditions. At 5 months, then alginate globules containing notochordal cells were fixed using cacodylate buffer modified from Petit et al. 1996 and processed for histological examination and stained with Toluidine Blue, Safranin-O, and for immuno-reactivity to collagen type II and aggrecan.

### Cell Morphometry:

Histomorphometric methods were used to evaluate the cultured cells. Total cell area measurements were performed using a Visiopharm Integrator System (Visiopharm Inc. Hørsholm, Denmark) using a Leica DM 4500 B microscope equipped with a motorized stage with 8-slide capacity and Olympus DP70 camera. 100 cells were counted from Safranin-O stained sections of both normoxic and hypoxic cultured cells using specific inclusion criteria that required the cells to have an intact cell membrane, visible nucleus and clear cytoplasm [20]. Each cell was individually masked and image analysis was performed to obtain total cell area. Visiopharm™ software was used for the analysis of cell area.

### Scanning Electron Microscopy:

Parallel notochordal cell cultures were grown in alginate globules removed, fixed and prepared for electron microscopy. Briefly, the alginate globule containing cells were immersed in primary fixative containing 4% Paraformaldehyde, 1% Glutaraldehyde in 0.1 M phosphate buffer pH 7.2 for 20 minutes. Next they are washed with 0.1 M Phosphate buffer (pH 7.2) 3 times and post fixation was performed using 1% Osmium Tetraoxide buffered with 0.1 M Phosphate pH 7.2 for 20 minutes and dehydrated through graded alcohols and dried using a critical point drier. The fixed globules were examined using Scanning Electron Microscopy (Hitachi S-3400 Variable Pressure Scanning Electron Microscope) at various magnifications and angulations of the stage in order to obtain the best imaging of the specimens.

### Immunohistochemistry:

Specimens were sectioned at 4 μm, mounted on silanized slides cleared using xylenes and alcohols and stained in parallel with positive and negative controls. The antibodies were optimized for concentration of primary antibody such that anti-collagen type II was stained at 1:800. The anti-aggrecan was obtained from a hybridoma supernatant and therefore was not as concentrated and was used at a 1:10 dilution. The immune-like reactivity was evaluated using avidin-biotin conjugated primary and secondary antibodies, visualized using diamino-benzidine methods (DAB kit-vecta stain) and counter-stained with haematoxylin. For positive control, articular cartilage, known to contain both aggrecan and collagen type II (bovine tarsal joints) was stained and the negative control was absent the primary antibody.

### Statistical Analysis

The data obtained from our histomorphometric analysis which consisted of 200 separate measurements was plotted and as suspected found that it was not normally distributed. Therefore non-parametric statistical methods were ised in order to analyze for statistical significance. The Mann-Whitney U test was used and it was found that the mean cell area for hypoxic cultured as compared to normoxic cultured cells was statistically significant at the p=0.0001 level.

### Results:

### Hypoxic and Normoxic Monolayer Cultures:

Canine non-chondrodystrophic notochordal cells thrive under hypoxic culture for at least 5 months, the time at which they were removed from culture and evaluated. There was no evidence of decline in cell growth within the hypoxic cultures at 5 months. The cultures were examined each three days as the medium was replenished and were photographed at least weekly throughout the 5 months of culture. Within one week cells appeared upon the bottom of the 6-well dishes initially as spherical/ovoid bubbly looking or “physaliferous” cells. Within two-weeks these cells had spread to become large complexes within both the normoxic as well as hypoxic conditions which in the case of the normoxic cultures became a completely confluent monolayer of cells by 4 weeks. However the hypoxic cultures exhibited a striking difference in appearance. Within one month large macroscopic structures formed upon the tissue culture plate surface in stark contrast to the more uniform monolayer appearance typical of the cells cultured under normoxic conditions. The hypoxic cultured cells spontaneously formed a circular structure that measured 2 mm in height and approximately 8 mm in diameter after 2 months in culture (FIG. 1 A). The construct was examined after fixation and sectioning and found to demonstrate robust Safranin-O staining, indicative of a proteoglycan-rich matrix (FIG. 1 B). During removal using forceps the construct demonstrated viscoelastic properties by resistance to compression and resumption of its oval shape after forceps compression was released.

After four-weeks in culture cells cultured under normoxia and hypoxia were evaluated using scanning electron microscopy (SEM) and found to have dramatic differences in appearance. After 4 weeks the cells grown under hypoxic conditions had developed large spanning ‘fibrils’ throughout the culture plates (FIG. 1 C) whereas the normoxic cultures had simply grown into a confluence of strictly monolayer fibroblastic-appearing cells (FIG. 1 D).

### Alginate Globules:

Notochordal cells contained within alginate globules demonstrated marked differences in behaviour under culture in hypoxia as compared to normoxia. The hypoxic cultures formed clusters of cells within two weeks that continued to develop over time. The hypoxic cultures formed dense cell clusters both within the alginate globule as well as many ‘buds’ of cell clusters upon the surface and within the alginate globule. Such behaviour never occurred within globules of cells cultured under normoxia (FIG. 2 A-B).

### Histological Analysis of Globule Cultures:

The cells cultured under hypoxia demonstrated a robust cellular and extracellular matrix, with large, well defined cells and intense metachromatic staining using Toluidine Blue in stark comparison to normoxic cultures (FIG. 2 C-D). Safranin-O staining was similar in pattern, cells cultured under hypoxia were robust and well-defined whereas the normoxic cultures were not confirming the superior histological properties of hypoxic culture as compared to normoxia such that cultures under hypoxia preserved an organized matrix and large cells similar to the in vivo setting and in stark comparison to normoxia (FIG. 2 E-G). The globules containing notochordal cells cultured under hypoxic conditions demonstrated strong immune-reactivity to collagen type II and aggrecan that was very similar in both the in vivo disc NP as well as the hypoxic cultures (FIGS. 3 A-F). However strikingly at 5 months there was no evidence of collagen type II and limited, poor aggrecan immune reactivity in the normoxic cultures.

### Histomorphometry:

In general the cells were much larger cultured under hypoxic conditions and showed intact cyto-morphology (intact cell membrane, visible nucleus, and clear cytoplasm-as well as some cells with matrix within the cytoplasm). Normoxic cultures, on the other hand, had far smaller cells, and many did not show the inclusion criteria. In fact, many of the cells did not appear viable as evidenced by a disorganized nucleus, fragmented cell membrane and fragmented/fibrillary appearance on Safranin-O and Toluidine Blue staining and the hypoxic conditions demonstrated significantly larger area than those cultured under normoxia (FIG. 4 A-C). Our morphometric analysis revealed a strikingly statistically significant loss of cell area for cells cultured under normoxic conditions as compared to those under hypoxia.

### Scanning Electron Microscopy:

Scanning electron microscopy (SEM) is an ideal technique with which to observe such structures from a morphological and topographical perspective, therefore in parallel with the histological and immunohistochemical staining, SEM was also performed. SEM is a high-resolution technique capable of providing clear morphological details of cells and tissues and it is able to show a 3D representation not possible using histological section. The ability of notochordal cells to self-assemble and to assume complex 3D configurations and whether such behaviour occurred in both normoxic and hypoxic conditions were of interest. A striking difference in cellular behaviour under our two different tissue culture conditions such that the normoxic cultures revealed nothing—no 3D structures or even any cellular remnants but fixed and denatured alginate whereas the hypoxic cultures formed a robust, complex 3-D tissue ‘construct’ with variable cellular formations including from fibrillary structures, complex cellular networks and the formation of stiffening adhesions between cells of the construct (FIG. 5 A-C) was found.

### Discussion:

### Hypoxia Drives Notochordal Self-Assembly:

Here it is demonstrated for the that notochordal cells thrive under hypoxic tissue culture for at least 5 months and develop an organized, complex 3D extracellular matrix rich in collagen type II and aggrecan; which are vital to the function of the disc nucleus. Parallel notochordal cell cultures harvested from identical sources but cultured under normoxic conditions (21% oxygen) reveal far smaller cells, many of which lack a cell membrane and nucleus and do not appear to survive this length of time in culture. Further, the matrix produced by these cells appears to be remnants of cellular debris and is quite distinct from the hypoxic cultured cells. In addition normoxic cultured cells do not demonstrate either collagen type II or aggrecan staining. Hypoxia appears to drive notochordal cells cultured in monolayer to form large fibrillary processes that interconnect cells growing upon the tissue plate surface. This phenomenon was never seen under normoxia. The ability of notochordal cells to ‘self assemble’ under hypoxic tissue culture conditions in both monolayer and 3D culture condition strongly suggests that notochordal cells retain their innate capacity to function as ‘organizer’ as well as structurally supporting cells.

### Developmental Aspects of Notochordal Cells:

During development and morphogenesis cells within the embryo migrate and differentiate along commitment pathways through a complex process termed convergence and extension-such as during gastrulation and neurulation [21]. This process of convergence and extension defines how cell populations narrow and lengthen during development and in so doing exert pressure within and around developing cells/tissues of the embryo. Tissues become ‘stiff’ at pivotal times during early development in order for the embryo to properly form. Ordered cellular migration and differentiation is typified by the notochord and its sheath which is one of the earliest areas where tissue stiffness develops in order to provide the longitudinal axis of the developing embryo.

### Developmental Aspects of Notochordal Cells:

It is the notochord that by the secretion of a complex series of signals during development acts as the principal ‘organizer’ of the developing embryo whereby local undifferentiated mesenchymal cells are induced to differentiate into organs and organized tissues. For example, a host of complex signaling pathways involving Sonic Hedgehog (SHH), Noggin, Chordin, BMP-4 and various transcription factors such as Sox5 and 6 are involved in the tightly controlled maturation of the primordial notochord to form mature notochordal cells as well as controlling the fate of paraxial mesodermal tissues [22]. Signaling from the notochord also induces the formation and migration of neural crest cells to become the neural tube and finally the spinal cord [23, 24].

The densely packed cells of the notochord and somatic mesoderm interact with one another and/or with the extracellular matrix between the cells whereby they undergo critical and complex changes in spatial regulation called ‘cellular intercalation’-inclusive of ‘protrusion’ [21]. Protrusions occur in parallel with the development of stiffening adhesions between neighboring cells that allows traction to be imposed upon each other thereby allowing them to produce forceful intercalation and tissue organization [25]. The ability to resist deformation while at the same time resist re-arrangement is accomplished by virtue of the cells developing in a tightly-knit configuration as well as the development of stiffening adhesions between the cells [21]. Stiffening adhesions are thought to develop along the anterior-posterior surfaces of the cell bodies and in so doing resist separation. At the same time they must also have the capacity to assemble and disassemble in concert with growth and re-organization [21]. Through the use of scanning electron microscopy it is found that notochordal cells organize into a 3D configuration and over time ‘bud’ within and out of the alginate globule containing the cells as they form a large, interconnected construct. Further, the presence of stiffening adhesions between cells of the tissue construct indicates that under hypoxic conditions, notochordal cells are able to self-assemble a complex 3D cellular and extracellular matrix that in many ways appears to recapitulate key aspects of development (FIG. 5).

### Cellular and Molecular Aspects of Hypoxia and Nucleus Pulposus Cells:

In order to survive within the hypoxic nucleus, NP cells have been reported to highly express GLUT-1 (glucose transporter protein), a protein that facilitates the entry and utilization of glucose in anaerobic metabolism [26, 27]. Another key factor in the hypoxic environment in many cells/tissues is the multifunctional and atypical growth factor ‘Connective Tissue Growth Factor’ (the current accepted terminology is ‘CCN-2’) which is up-regulated by hypoxia through Hif-1-dependent (Hypoxia inducible factor) and TGF-β1-independent pathways [28]. CCN-2 expression is regulated by HIF-1α and its expression in many cells is increased under hypoxic conditions [28]. It is HIF-1α that regulates such mechanisms as glucose transport, angiogenic factors (where vasculature is present) such as VEGF as well as various genes involved in cell cycle regulation and apoptosis [29]. CTGF is a multi-functional molecule known to be involved in a host of cellular processes such as cell proliferation, differentiation, matrix deposition and remodeling, acquisition of tensile strength in cartilage due to its importance in the production of aggrecan and link protein and may have anti- or pro-apoptosis depending upon the cells and tissues involved. Further, CTGF is known to often be a down-stream effector molecule of TGF-β signaling [6, 30, 31]. A deficiency of CTGF leads to skeletal dysmorphisms secondary to impaired chondrocyte proliferation and extracellular matrix composition such that CTGF-deficient mice die shortly after birth due to severe skeletal dysplasia [32]. It has been reported that cells deficient in Hif-1α are incapable of inducing the expression of CTGF mRNA after exposure to hypoxia [28]. This lack of hypoxic activation in Hif^{-/-}1α animals suggests that the accumulation of extracellular matrix conferred by CTGF downstream of TGF-β signaling may be mediated by hypoxia responsive genes. It has been previously demonstrated that notochordal cells secrete CCN-2/CTGF, and that the addition of recombinant CTGF increases aggrecan gene expression by NP cells indicating that intervertebral disc-derived notochordal cells are capable of directing the function other cells [10]. With respect to the human condition, it has been reported that HIF-1α is more strongly expressed by nucleus pulposus cells in herniated human discs than normal discs suggesting an attempt at recovery/repair-however there is no explanation for the mechanism at play under these circumstances [31, 32].

It has been reported that rat nucleus pulposus cells increase their expression of the classic chondrogenic genes collagen type II, aggrecan and Sox9-genes when cultured for 36 hours under hypoxia providing further evidence of the beneficial effect of hypoxia [33]. In this study it was observed that hypoxia activates Akt signaling, a factor considered important in the maintenance of Glut-1 transcriptional activity and therefore anaerobic delivery of glucose to the cells. Further, activation of MEK1/ERK pathways by hypoxia was reported to confer protection from apoptosis in NP cells induced by hypoxia and the products of the activation of these pathways may be one mechanism whereby NP cells adapt to their environment [33].

Notochordal Cells Cultured Under Hypoxic Conditions Produce a Matrix Indistinguishable from the In Vivo Setting:

The non-chondrodystrophic (NCD) canine is characterized by its resistance to the development of degenerative disc disease (DDD) and it is the intervertebral disc (IVD) of this animal that is characterized by its retention of an almost pure population of notochordal cells. This appearance is stark contrast to the chondrodystrophic (CD) canine that has a much more fibrocartilagenous IVD with an abundance of chondrocyte-like cells and few notochordal cells. It has been demonstrated that notochordal cells of the NCD canine secrete a form of aggrecan that is superior to that formed by the CD notochordal cells in that the aggrecan formed by NCD notochordal cells assembles into aggregates further away from the cell surface and in so doing allows for a superior hydrophilic capacity [34]. Owing to the resistance to DDD and greater biomechanical load-bearing capacity of the NCD IVD that it has been postulated that the IVD that is rich in notochordal cells may confer superior biomechanical properties as compared to the more fibrocartilagenous disc [35].

The results of this study suggests that future strategies focused upon cell-based therapies particularly involving notochordal cells should strive to culture such cells under hypoxic conditions since normoxia over the long term does not support cell survival and matrix deposition. Further, hypoxia seems to provide the single necessary component to activate NP and/or notochordal cells to proliferate and produce an abundant, biologically relevant extracellular matrix and to undergo self-assembly-akin to that very process which occurs during development. Understanding the biological mechanisms underlying the robust responses of notochordal cells to hypoxic tissue culture, the capacity of notochordal cells to stimulate other NP cells and particularly the role(s) played by reactive oxygen species will allow for future mechanistic investigations of the responses of these cells to low oxygen environments. It may be that notochordal cells act as a kind of regenerative reservoir capable under the right conditions of inducing restorative processes that could potentially be harnessed in a future cell-based therapy for the treatment of degenerative disc disease.

### Example 4

### Notochordal Cells Protect Nucleus Pulposus Cells from Degradation and Apoptosis: Implications for the Pathophysiology and Treatment of Intervertebral Disc Degeneration

### Introduction:

Degenerative disc disease (DDD) is an extremely common and expensive healthcare ailment which to date has no curative strategy [1]. Given the lack of a biological “regenerative” strategy for the treatment of DDD, a therapeutic intervention that may offer restorative qualities to the disc is a much needed and widely sought goal. The ideal biological agent would re-activate homeostatic mechanisms innately inherent to the healthy intervertebral disc (IVD) that are known to decline and ultimately fail with progressive degeneration. The capacity to re-establish equilibrium between catabolic and anabolic tissue remodeling would represent the ideal regenerative strategy for the treatment of DDD.

With respect to the pursuit of potential biological therapies, lessons learned from the study of the non-chondrodystrophic (NCD) canine IVD might provide essential molecular clues in the quest to restore homeostasis to the disc. The NCD canine is unique amongst the canine sub-species in that this animal is protected from the development of degenerative disc disease (DDD) and preserves its notochordal cell populations throughout most of its life [36, 37]. It has been previously determined that notochordal cells obtained from non-chondrodystrophic canine intervertebral disc nucleus pulposus (NP) secrete Connective Tissue Growth Factor (CTGF/CCN-2) a relatively new, though incompletely understood, growth factor-like molecule. Moreover, conditioned medium obtained from these cells up-regulates important matrix gene expression, cell proliferation and proteoglycan production [38, 39].

Additionally, the culture of notochordal cells under hypoxic conditions has been recently explored since these cells have adapted to survive within such conditions in vivo. It is reported that under hypoxic tissue culture conditions notochordal cells assemble complex three-dimensional tissue constructs that recapitulate important histological characteristics of the in vivo disc nucleus pulposus [40]. This work and that of other investigators has thus supported an emerging body of evidence indicating that notochordal cells confer anabolic capacity upon NP cells and that their absence is associated with vulnerability to degenerative change [36, 38, 39].

DDD in humans is known to involve the progressive degradation of the extracellular matrix and loss of viable NP cells. Under degenerative conditions there is increased activity of inflammatory cytokines, in particular IL-1β, as well as increased expression of the death receptor Fas/CD95 and activity of Fas-Ligand (FasL) [41, 42]. The main mediators of the catabolic processes involved with DDD are IL-1β and FasL that result in enzymatic degradation primarily by ADAMTS and MMP family members. The NP cellular and extracellular matrix (ECM) is a tightly regulated environment where homeostasis is maintained by the matrix metalloproteinases (MMPs), ADAMTS family of enzymes (A disintegrin and metalloproteinase with thrombospondin motifs) and tissue inhibitors of metalloproteinases (TIMPS). These enzymes function in a similar fashion to those at play in skin and cartilage and in an integrated fashion, balance anabolic and catabolic processes within the framework of ongoing tissue remodeling [43, 44]. Dysregulation of the activity of the MMPs, ADAMTS's and TIMP enzymes results in increased catabolic activity and progression of DDD largely due to the influence of inflammatory cytokines, in particular IL-1β and the cell surface ‘death receptor’ Fas/CD95 [45, 46]. Of note, the inflammatory cytokine IL-1β is widely regarded as a key factor in the progressive degeneration of the IVD NP [46]. In addition it has been demonstrated that FasL may sensitize disc cells to II-β mediated apoptosis; resulting in an synergistic action upon disc cells that increases with further degeneration [46].

The ability to mitigate DDD by controlling the otherwise progressive loss of viable cells and ECM within the IVD NP would be a giant step in the treatment of this progressive disorder. The unique resistance DDD demonstrated by the NCD canine when taken in context with recently demonstrated anabolic effects conferred by notochordal cells strongly suggests notochordal cells in addition to providing anabolic stimulus may also confer protection from cytokine and Fas-mediated degradation and cell death. Here for the first time it is demonstrated that NCCM is capable of protecting NP cells from exactly these processes in vitro. The ability to harness the restorative properties of the notochordal cell may lead to novel, molecular therapies in the treatment of degenerative disc disease.

### Methods and Materials:

Non-Chondrodystrophic Canine Discs: Dogs were obtained in collaboration with the University of Guelph, School of Veterinary Medicine (Guelph, Ontario, Canada). All animals were 8-12 months of age and had failed at adoption and ethics approval was in accordance with the animal care policies of the Toronto Western Hospital.

Tissue Harvest: Deep sedation was achieved using a combination of Acepromazine (Atravet-Aerst pharmaceuticals 10 mg/mL) mixed with Xylazine 100 mg/mL (Xylomax-Bimeda-NHC Animal health) at a combined dose of 1 mL/15 kg body weight. Once deep sedation had occurred euthanasia was accomplished using intravenous sodium pentobarbital (Euthansel-Sherring Blain) at a dose of 0.30 ml/kg body weight.

Tissue Culture Conditions: Within one hour of euthanasia the lumbar spine was removed from 6 dogs and after careful removal of soft tissues the spines were liberally washed with distilled H₂0, rinsed with Clidox™ and then covered with Betadine™ and allowed to soak for 60 minutes at 4° C. Then under aseptic conditions 6 nuclei pulposi were removed from the intervertebral discs of each animal and the disc tissues transferred to a sterile container filled with Advanced Dulbecco's Modified Eagle Medium/F-12 (ADMEM/F-12) supplemented with 100 Units penicillin/streptomycin. Any visible annulus fibrosus was meticulously removed from the dissected nucleus pulposus tissues and following two washes with complete medium the tissues were sequentially enzymatically digested over night according to established methods previously reported [38-40]. The following day the cells were filtered with a 70 μm cell strainer (Falcon) and found to be virtually 100% notochordal in content, recognized by the classic physaliferous, large vacuolated appearance of the cells. The cells were then counted and placed into 1.2% sodium alginate beads according to established methods and placed under hypoxic tissue culture (3.5% O₂, 37° C. and 5% CO₂) with Advanced DMEM/F-12 (ADMEM/F12) supplemented with 8% Fetal Bovine Serum (FBS) and 100 Units penicillin/streptomycin for two days. After two days the alginate beads containing the notochordal cells were thoroughly washed with PBS changes each 15 minutes for two hours and the medium replaced with FBS-deficient ADMEM/F-12 containing 100 Units penicillin/streptomycin and returned to hypoxic culture for three days. Three days of hypoxic culture resulted in the development of notochordal cell conditioned medium (NCCM). Bovine NP cells had been obtained earlier from caudal discs of 3-year old steers sourced from a local abattoir according to our established methods [4-6] and once enzymatically digested, filtered and counted were plated under hypoxic conditions in T-75 flasks (CoStar) in ADMEM/F-12 with 100 Units PSF and 8% FBS. The cells were allowed to expand to 70% confluence and were harvested and flash frozen using 90% FBS and 10% DMSO and stored in liquid nitrogen. The day that the canine notochordal cells were harvested, the bovine NP cells were thawed and following washing with complete medium the cells were plated and allowed to expand to 70% confluence over the three days of NCCM development. On the day that development of NCCM began the bovine NP cells were trypsinized and re-seeded at 0.5×10⁶ cells/well in 6-well plates and placed under hypoxia and allowed to expand for 3 days.

Induction of Apoptosis: 10 ng/mL recombinant bovine IL-1β (Thermo Scientific RP-87269) or 10 ng/mL IL-1βplus 10 ng/mL recombinant human Fas-ligand (Fas-L) (Alexis Biiochemical Alx-522-001) were added to the bovine NP cultures simultaneously with the addition of either 3 mL NCCM (previously filtered through 0.2 μm cell syringe tip filter (Costar) just prior to use) or basal medium in order to induce apoptosis. Basal medium alone was used as a control in which the bovine NP cells once expanded were cultured for the three-day experimental period with basal medium only. Basal medium consisted of Advanced DMEM/F-12 supplemented with PSF but no FBS under hypoxic conditions (3.5% O₂) and 95% humidity at 37° C. The cultures were then placed back into hypoxic tissue culture for 48 hours in which there were four experimental groups of NP cells treated with 1: IL-1β only; 2: IL-1β+Fas-L; 3: IL-1β+Fas-L+NCCM and 4: basal medium alone.

Evaluation of Cell Death: Flourescence Activated Cell Sorting (FACS) to (Becton Dickinson Dual Laser FacsCalibre) were used to evaluate the total extent of cell death and apoptosis by incubating the cells in appropriate buffers and labeling with Propidium Iodide (PI) and Annexin-V respectively, according to the manufacturers instructions. All treatments were performed in triplicate with cells incubated for 48 hours under hypoxia. After 48 hours the cells from each well were collected individually by trypsinization (both suspended/dead cells plus monolayer cells were all collected for total cell recovery) washed in ice-cold PBS and re-suspended in 1 ml Binding buffer (Annexin-V apoptosis detection kit, K101-100, Cedarlane). In order to establish a positive control untreated cells in DH₂0 were resuspended for 15 minutes prior to using binding buffer. ‘The Cell Quest’ (BD Biosciences) was used to analyze the percentage of Annexin-V (apoptotic) and PI (necrotic cells) amongst total cells. To each sample Annexin-V (AV) and PI was added according to manufacturer's instruction and incubated at room temperature in the dark for 10 minutes. The cell harvest was filtered using 70 μm cell strainers (Falcon) just prior to flow cytometric analysis for which “FacsCalibre” was used. Data analysis was performed using CellQuest Pro. adjusted the initial scatter plot to avoid small cells or sub-celluar debris and then subsequent gating around dead cells (Annexin-V and PI positive cells) determined from analysis of cells treated with distilled H₂0. The experimental data was obtained using the gating protocols developed from the positive (distilled H₂0) and negative (untreated cells) controls. Dead/necrotic cells were interpreted as PI positive and Annexin-V negative and cells undergoing apoptosis as both PI and Annexin-V positive). Statistical evaluation of compared samples was performed using T-tests.

Gene Expression Data: The effect that NCCM had upon NP cells in terms of protection from both cell death and matrix degradation was of interest. Therefore, real-time quantitative RT-PCR (qRT-PCR) technology was used to examine the expression of the extracellular matrix genes aggrecan, collagen type II and the cell-surface glycoprotein hyaluronic acid assembly site; CD44 and link protein. The expression of genes encoding matrix protecting factors such as TIMP-1 as well as the principal matrix degrading enzymes MMP-3 and ADAMTS-4 was also of interest. Some primers used were obtained from data presented in previously published peer reviewed journals after verifying the sequences and primer design against established databases such as PubMed [47]. All other primers were designed using Primer Express 3.0 according to standard guidelines, based on bovine coding sequences of each gene of interest (Table 1). Each primer set was optimized to determine the optimal primer concentration, annealing temperature and cDNA concentration. Total RNA was extracted from cells using Trizol reagent (Sigma) according to manufacturer's recommendations. RNA quality, purity and concentration were determined with Nanoprop (ND-1000, V3.5). For cDNA synthesis reverse transcription was performed on 1 μg of total RNA sample using SuperScript II based reverse transcription system (Invitrogen). The real-time PCR analysis was performed using iCycler iQ5 real-time PCR detection system (BioRad Laboratories) and iQ SYBRgreen Supermix (BioRad laboratories). Primer sets with equitable efficiencies were determined based on cDNA standard curves were used for quantitative real-time PCR analysis using the delta-delta-Ct method [48]. The expression of the target genes was normalized using the constitutively expressed ‘housekeeping’ gene hypoxanthine guanine phosphoribosyltranferase (HPRT). The threshold for determining significant changes in the normalized expression of genes of interest was established at a 1.5 fold difference in comparison with control levels.

qRT-PCR Optimization: Each primer pair was optimized for both optimal primer concentration and amount of cDNA in order to determine that amplification was within 15-25 amplification cycles. By this approach, non-linear/endpoint amplification data were avoided. In order to minimize the formation of primer-dimers, the melting curves generated by the PCR procedure for each primer pair were carefully examined in order to ensure a uniform loss of fluorescence at the desired melting point. The expression profile for each primer pair used was also evaluated to ensure that there were no aberrant peaks present during the amplification phase of the PCR reaction; this step is helpful to ensure that there were no primer-dimer or self-annealing artifacts that could be mistaken for real gene expression.

### Results:

Protection from Apoptosis: Annexin-V and Propidium Iodide (PI) were used to evaluate for apoptosis and cell death. Annexin-V is a protein that preferentially binds to phosphatidyl serine (PS) which undergoes specific changes during apoptosis and when conjugated to fluorescein isothiocyanate (FITC) it is capable of detecting early apoptosis via fluourescence imaging. PI is capable of entering the cell only in the instance of disrupted plasma membrane and is a non-specific marker of cell death often used to in connection with Annexin-V to discriminate apoptosis vs necrotic cell death. Annexin-V-FITC and PI labelled cells were quantified using FACS allowing discrimination between intact cells (FITC⁻PI⁻), early apoptotic (FITC⁺PI⁻) and late apoptotic or necrotic cells (FITC⁺PI⁺). Dead and dying cells were then expressed as a percentage of Annexin-V-FITC or PI positive cells divided by the total number of dead cells. It was found that NCCM statistically significantly protects NP cells from both total cell death and apoptosis as compared to cells treated with II-1β or II-1β+Fas-L such that NCCM in the presence of IL-1β+Fas-L restored the percentage of apoptotic cells to base-line levels with a 50% reduction of total cell death (FIGS. 6 and 7). Our preliminary experiments indicated that IL-1β alone was not sufficient to induce apoptosis or significant cell death within the 48-hour experimental period but required the addition of Fas-L in order to induce detectable apoptosis.

### Impact of NCCM on Genes Encoding Structural Proteins and Modulators of the Extracellular Matrix

Structural protein gene regulation: The use of NCCM in cultures co-treated with IL-1β however had a dramatic effect upon both genes that encode structural proteins as well as the inhibitors and activators of extracellular matrix remodeling (FIG. 8). The results were adjusted relative to basal conditions where the cells were treated with ADMEM with no FBS supplementation (0% FBS) (FIG. 8). For the most part the changes in gene expression induced by the addition of IL-1β+FasL were similar to IL-1β alone but in some cases the addition of FasL caused a more pronounced effect. Consistent with past reports, aggrecan gene expression was decreased with the use of II-1β and IL-1β+FasL [45, 46, 49, 50]. However the use of NCCM resulted in a preservation or mild up-regulation of aggrecan gene expression. Also, the expression of the gene encoding link protein, the proteins that stabilize the aggrecan molecule to hyaluronic acid is markedly down-regulated by II-1β and IL-1β+FasL yet it is mildly up-regulated by the use of NCCM. In addition, the expression of the gene encoding the CD44 receptor is markedly up-regulated by NCCM compared to the level induced by IL-1 and IL-1β+FasL. II-1β and IL-1β+FasL resulted in the down-regulation of collagen type II but this down-regulation was strongly attenuated in the presence of NCCM.

Extracellular Matrix Remodeling Genes: ADAMTS4, the major aggrecanase involved in reversible aggrecan core protein degradation was up-regulated by II-1β and IL-1β+FasL but is increased almost 3-fold more by the use of NCCM (FIG. 9). The expression of the major collagenase that irreversibly degrades collagen, MMP3, is markedly up-regulated by II-1β and IL-1β+FasL but this increase in catabolic enzyme gene expression is blunted by NCCM. The MMP inhibitor TIMP-1 was down-regulated in the presence of II-1β and IL-1β+FasL however the gene expression of this inhibitor of MMP activity is up-regulated by NCCM (FIG. 9). Taken together it is seen that NCCM stimulates NP cells to mount a strong defense against IL-1β and IL-1β+Fas-L mediated degradation and in particular NCCM confers an anti-apoptotic effect upon NP cells (FIG. 10).

Therapeutic Molecular Agents to Restore NP Cells:

It has been have previously determined that notochordal cells produce a relatively new and atypical growth factor ‘Connective Tissue Growth Factor’ also termed CCN-2. Further, it has been determined that recombinant CTGF/CCN-2 is capable of up-regulating the expression of the gene encoding for the proteoglycan aggrecan. CTGF/CCN-2 is well known to be intimately involved with a host of connective tissue-related events and can be either pro or anti-apoptotic depending upon the conditions, cells and environment. Initial in vitro experiments were performed using recombinant human CTGF (rhCTGF) in the presence of IL-1,3-treated NP cells and have found (using FACS analysis; Propidium Iodide and Annexin-V labelling) that 10 ng/mL rhCTGF confers an anti-apoptotic effect similar to what we have observed using NCCM (FIG. 11).

### Discussion:

The hallmark of progressive DDD that leads to cell death and ECM degradation is an inability of NP cells to maintain normal homeostatic tissue remodeling under the influence of increased cytokine and death receptor expression. This has led to the notion that IL-1 receptor antagonists and/or blocking the IL-1 signaling pathway may be attractive therapeutic options however currently no such treatment exists [41, 42, 45]. With a view to solving the problem of progressive DDD the purpose of this study was to determine the ability of notochordal cell conditioned medium to inhibit apoptosis of NP cells and to resist gene expression changes induced by IL-1β and FasL.

An essential component of normal homeostatic extracellular matrix turnover is the reversible degradation of the aggrecan core protein controlled to a large degree by ADAMTS-4 [37, 51, 52]. Aggrecan degradation is a pivotal, though reversible, event in that aggrecan is largely responsible for the disc's compressive stiffness and tremendous swelling pressure and its turnover is slow and tightly regulated [3, 11]. However, under the influence of IL-1β and/or FasL as well as some members of the MMP family (notably MMP3) unbalanced degradation of the aggrecan molecule leads to disruption of aggrecan, impaired water-binding within the disc nucleus milieu and secondary to further MMP action leads to irreversible degradation of collagen type II and subsequent matrix failure [45, 50]. The ability to protect NP cells from accelerated apoptotic cell death and the ECM from degradation would be a major step toward a biological treatment for DDD. Here for the first time it is demonstrated that soluble factors secreted by notochordal cells protect NP cells from IL-1β and FasL induced degeneration and cell death.

Mechanisms of NP degeneration: Central to the theory that DDD represents a loss of homeostatic control over the IVD NP it has been demonstrated that the NP undergoes typical internal disorganization accompanied by degenerative changes in the annulus commensurate with a reduction in the number of notochordal cells within the NP [37, 53]. The loss of viable NP cells results in dysregulation over tissue remodeling and renewal as a consequence of impaired cellular and extracellular matrix maintenance. It follows that the progressively acellular and degenerative NP becomes deficient in essential extracellular matrix molecules and growth factors, available receptor sites and other signaling molecules resulting in the failure of the NP as a functional organ [37, 53-55]. The crucial role played by IL-1β has been explored by a number of investigators and it has been demonstrated that the treatment of human IVD cells with this inflammatory cytokine up-regulates the expression of genes responsible for NP degradation and suppresses the genes responsible for anabolic activity [10]. This pro-catabolic activity, when unmatched by a balanced anabolic-repair mechanism, creates a loss of tissue homeostasis and represents the pivotal ‘switch’ in the progressive degenerative cascade [45, 55-57].

The expression of the cell surface ‘death receptor’ Fas-L/CD95 has been shown to increase in disc herniations with increasing age and degree of degeneration [58]. Also, increased number of Fas/CD95 positive cells in a rat DDD has been demonstrated indicating the likely contribution of this signaling pathway to increased cell death associated with progressive degeneration [59]. It has been shown that IVD cells pre-treated with IL-1β increase their rate of apoptosis in response to co-treatment with FasL suggesting a synergistic activity between these two mediators of cell death and extracellular matrix degradation [46]. A close relationship was observed between the effects of IL-1β and IL-1β+ Fas-L in terms of gene expression whereby IL-1β and Fas-L that in general up-regulated genes associated with ECM catabolism and down-regulated anabolic-related genes. However, the addition of Fas-L was necessary in order to induce significant apoptosis and cell death suggesting that an association between these effectors of cell death and degradation is a distinct possibility.

The action of IL-1β and FasL upon cellular viability is clearly of paramount importance to the progression of DDD since degeneration is not only associated with the number of viable cells present within the nucleus but also to the declining ability of the cells to manufacture and maintain the abundant extracellular matrix (ECM) [53, 54]. The nucleus pulposus is composed of a complex arrangement of molecules that serve to maintain the viscoelastic properties and high swelling pressure associated with the NP. Among these molecules are many species of proteoglycans and collagens within the NP; however, aggrecan and collagen type II figure prominently with respect to essential load-bearing and water-binding function [60]. Aggrecan is the principle proteoglycan linked to long chains of hyaluronic acid via their G1 globular domains and stabilized upon the hyaluronic acid monomer by link proteins [37, 61-63]. Aggrecan is a complex molecule with a high net negative charge that due to its hydrostatic interactions is capable of binding water molecules tightly and contributes substantially to the compressive resistance and viscoelastic properties essential to normal function of the IVD. With respect to collagen content, the NP contains an abundance of type II collagen in contrast to the annulus that has a higher component of type I collagen [54]. However with progressive degeneration, there is a shift towards an increase in collagen type I synthesis and a decline in the percentage of functional collagen type II in favour of increased type I formation within the NP leading to disordered biomechanical properties [54].

Nucleus Pulposus Extracellular Matrix Regulation: Just as with any connective tissue, the maintenance of homeostasis is dependent upon balanced degradation and repair. Degradation of the NP ECM is accomplished in a similar fashion to tissues such as skin and cartilage whereby the activity of matrix metalloproteinases (MMPs) and the ADAMTS family of enzymes (A disintegrin and metalloproteinase with thrombospondin motifs) work in a tightly regulated fashion along with inhibitors of their actions-tissue inhibitors of metalloproteinases (TIMPS) [43, 44]. Dysregulation of MMPs, ADAMTS's and TIMP maintenance contributes to the progression of DDD and is to a large extent due to the influence of inflammatory cytokines in particular IL-1β plus the cell surface ‘death receptor’ Fas/CD95 [45, 46]. MMPs are transcriptionally controlled by enzymes, growth factors and cytokines and are tightly regulated in the remodeling of the extracellular matrix as well as their involvement in other regulatory mechanisms including embryogenesis, organ morphogenesis and other aspects of tissue remodeling [56, 64-66]. MMP3 or ‘Stromelysin’ or MMP-3 is a major collagenase involved in the irreversible degradation of collagen and is increasingly expressed by NP cells contained within herniated and degenerative discs [44, 67, 68]. Le Maitre et al. reported that MMPs-1, -3. -7 and -13 are all up-regulated with progressive degeneration of the IVD NP and implicated IL-1β as a key molecule in intervertebral disc degeneration and hypothesized that inhibition of IL-1 activity may be an attractive therapeutic target [44].

A recent anatomical study by Pockert et al. evaluated human disc tissue for gene expression and protein level expression of various ADAMTS species and MMPs as well as expression of their inhibitors [50]. In this study these authors contend that there is a kind of two-step process at work whereby the ADAMTS family of aggrecanaseses notably ADAMTS-4 in the disc NP functions in a reversible fashion to degrade the aggrecan core protein. The activity of the MMPs however result in further degradation of the ECM by the irreversible breakdown of collagen networks within the IVD NP (predominantly collagen type II) [50]. This combined loss of aggrecan and collagen type II function lead to the subsequent loss of disc height and viscoelastic properties of the NP. Pockert et al. further speculate that the identification of the key enzyme(s) involved in protecting the IVD NP from aggrecan loss could lead to a biological therapy and perhaps avoid salvage surgical procedures that occur with advanced degenerative change. Of the ADAMTS family, ADAMTS4 is the major aggrecanase involved in the reversible degradation of the aggrecan core protein and it is known to be active in the IVD NP unlike articular cartilage where ADAMTS5 is the major aggrecanases [45].

Opposing ECM degradation by the MMPs are the Tissue Inhibitors of Matrix Metalloproteinases (TIMPs). Tissue inhibitors of matrix metalloproteinases (TIMP family) bind to MMPs and act to protect proteoglycan molecules from degradation by members of the MMP and ADAMTS family. Various members of this class of molecules are known to affect cell proliferation, survival and apoptosis and inhibit extracellular matrix degradation [64]. It has been reported that conditioned medium obtained from porcine chondrocytes treated with IL-1 had more than 90% of its MMP activity inhibited by the addition of recombinant human TIMP-1 or TIMP-2 [69]. Further, Bonassar et al. reported that TIMP-1 reduced the loss of sulfated glycosaminoglycans (GAGS) by 40% over 8 days of culture when added to bovine cartilage explants [65]. In a study demonstrating the anti-catabolic activity of the TIMP family of enzymes, Ellis et al. reported that both TIMP-1 and TIMP-2 completely abrogated the release of fragments of collagen from bovine nasal cartilage that had also been exposed to IL-1α [70]. TIMP-1 is one species within the larger family of matrix metalloproteinase inhibitors that are known to affect cell proliferation, survival and apoptosis and inhibit extracellular matrix degradation [64]. Also TIMP-1 is known to be up-regulated by IL-1β and IL-6 as well as several growth factors such as TGF-β and EGF [71]. Interestingly and of significance to this study is the report by Tan et al. whereby TIMP1 was found to suppress apoptosis in lymphoma cell lines as well as excitotoxic cell death in neurons [72]. The anti-apoptotic effect of TIMP-1 has been verified in a TIMP-1 knock-out mouse model by Davidson et al. whereby TIMP-1^{-/-} mice are much more sensitive to chemotherapeutic-induced apoptosis than are wild-type mice that express TIMP-1 thereby validating that active TIMP-1 confers an anti-apoptotic effect upon some cells [73]. In addition TIMP-1 pro-cell survival activity has been reported in human keratinocytes and fibroblasts and is considered to be one of the principal growth factors present in human serum [43]. Furthermore, Le Maitre et al reported that TIMP-1 and TIMP-2 are up-regulated in DDD suggesting that these particular MMPs are specifically involved with the mediation of DDD within the IVD [44]. It is due to the contribution to the turnover of the ECM that the net balance between TIMP/MMP activities are considered to be a pivotal element in the pathogenesis of degenerative disease such as arthritis and inflammatory-related disorders of the central nervous system such [64].

Relevance of the Canine Species: It is known that non-chondrodystrophic canines do not develop DDD or if at all, not until much later in life and that these animals maintain an abundance of cells of the notochordal phenotype within their IVD NP [36, 37, 74, 75]. Chondrodystrophic canines on the other hand which have far fewer notochordal cells and a more fibrocartilagenous appearance are prone to early DDD and also suffer spontaneous disc injuries [36, 74, 75]. Our central hypothesis is that notochordal cells, when present, confer matrix protection and homeostasis upon the NP therefore it follows from this series of experiments that notochordal cells may at least partially confer homeostatic balance by the suppression of apoptotic signaling induced by IL-1 and IL-1+FasL amongst NP cells.

Impact of Notochordal Cell Conditioned Medium: In our experiments in the absence of NCCM there is a clear matrix-degrading effect in all genes studied as well as in cell death/apoptosis assays. This clear catabolic effect is contrasted by the opposite effects seen with cells treated by IL-1β+ FasL in the presence of NCCM. For example, IL-1β+FasL-treated NP cells demonstrate marked apoptosis and cell death as well as down-regulation of the genes encoding aggrecan, link protein and TIMP-1. In addition these cells demonstrate up-regulated expression of the genes encoding the matrix degrading enzymes ADAMTS4 and MMP-3. IL-1β+FasL treated NP cells also increase the expression of the gene encoding the cell surface receptor CD44 where hyaluronic acid assembly occurs, a characteristic alteration in CD44 receptor expression that has been demonstrated in chondrocyte biology and is thought to reflect attempts by the cell at mediating inflammation and attempts at repair [76]. In summary, IL-1β+ FasL treatment of NP cells induces matrix-degrading genes and suppresses anabolic extracellular matrix assembly-related genes consistent with the literature concerning the action of IL-1β+ FasL upon NP cells.

The significantly increased cell death and ECM degradation noted in NP cells treated with IL-1β+FasL is markedly contrasted by NP cells cultured under identical IL-1β+FasL conditions but using NCCM rather than basal medium. In this case there is a maintenance or even mild up-regulation of the important extracellular matrix-related genes such as aggrecan, link protein and collagen type II and a down-regulation of the matrix-degrading enzyme MMP3. Consistent with the hypothesis that NCCM confers extracellular matrix homeostasis and mitigates against the degeneration-inducing effects of IL-1β there is also increased expression of the ADAMTS4, and more robust increase in both TIMP-1 and CD44 gene expression.

Notochordal cells confer a homeostatic-regulatory effect upon IL-1β+FasL-treated NP cells. The robust effect conferred by NCCM upon IVD NP cells strongly suggests that notochordal cells act as matrix ‘guardians’ that continue to secrete growth and other factors therefore modulating a homeostatic protective action on the disc nucleus [36, 38, 39, 77, 78]. It has been previously reported that conditioned medium obtained from notochordal cells (NCCM) increases the production of proteoglycan by nucleus pulposus cells (NP cells) and in a dose-dependent fashion and NCCM induces a modest non-dose dependent increase in cell proliferation [38]. Additionally, i NCCM induces NP cells to increase the expression of the aggrecan, versican and hyaluronic acid synthase-2 genes and that at least one important anabolic factor contained within NCCM is the atypical growth factor ‘Connective Tissue Growth Factor (CTGF/CCN-2) [39]. It is demonstrated herein that 100-200 ng/mL of recombinant human CTGF results in comparable up-regulation of aggrecan gene expression in NP cells cultured within alginate beads to that seen using NCCM suggesting that this molecule may be an important player in the soluble factor milieu secreted by notochordal cells within the IVD NP [39]. The potential restorative capacity of the notochordal cells has been explored by using in vivo studies whereby the notochordal cell-rich disc nucleus has been re-implanted in an animal with a damaged disc and afforded protection from degeneration [79, 80]. Notochordal cells may provide the essence of what is necessary to develop and maintain a healthy nucleus pulposus matrix given that NCCM is capable of mediating the otherwise clearly pro-apoptotic and matrix degrading effects of IL-1β and FasL

The preservation of aggrecan gene expression and the down-regulation of MMP-3 in the presence of the inflammatory cytokine IL-1β and FasL strongly suggest that NCCM contains powerful anti-apoptotic and anti-catabolic qualities. In the presence of IL-1β and IL-1β+Fas-L, NCCM protects against the down-regulation of aggrecan, link protein and collagen type II. When taken together with the down-regulation of MMP3 (a major collagenase) and up-regulation of one of the significant inhibitors of MMP activity TIMP-1, NCCM appears to provide a matrix-protective environment for NP cells.

### Therapeutic Molecular Agents to Restore NP and Cartilage Cells:

It has been previously determined that notochordal cells produce a relatively new and atypical growth factor ‘Connective Tissue Growth Factor’ also termed CCN-2. Further, recombinant CTGF/CCN-2 is capable of up-regulating the expression of the gene encoding for the proteoglycan aggrecan. CTGF/CCN-2 is well known to be intimately involved with a host of connective tissue-related events and can be either pro- or anti-apoptotic depending upon the conditions, cells and environment. In vitro experiments using recombinant human CTGF (rhCTGF) in the presence of IL-1β-treated NP cells and have found (using FACS analysis, Propidium Iodide and Annexin-V labelling) that 10 ng/mL rhCTGF confers an anti-apoptotic effect similar to what was observed using NCCM (FIG. 11).

The progressive loss of NP cell viability is fundamental to the progressive degenerative cascade at work in the phenomenon of DDD and these changes are also fundamental to the degenerative changes at work in articular or hyaline cartilage. These observations suggest that CTGF/CCN-2 could be an attractive therapeutic molecule to be used to suppress the apoptosis of NP cells. the presence of CTGF within NCCM can be detected for example using LC-MS/MS mass spectroscopy.

The results provided herein provide hypoxic in vitro evidence that notochordal cell-secreted soluble factors provide molecular signals that mediate cell death and degradation of NP cells induced by IL-1β and IL-1β+Fas-L.

TABLE 1
Real-time RT-PCR Primers.

| | | Accession | Sequence |
|---|---|---|---|
| Gene | Primer Sequence | Number | ID No |
| Aggrecan F | 5′-CCTGAACGACAAGACCATCGA-3′ | U76615 | 1 |
| Aggrecan R | 5′-TGGCAAAGAAGTTGTCAGGCT-3′ | | 2 |
| Collagen type II F | 5′-AAGAAGGCTCTGCTCATCCAGG-3′ | X02420 | 3 |
| Collagen type II R | 5′-TAGTCTTGCCCCACTTACCGGT-3′ | | 4 |
| MMP3 F | 5′-CACTCAACCGAACGTGAAGCT-3′ | AF135232 | 5 |
| MMP3 R | 5′-CGTACAGGAACTGAATGCCGT-3′ | | 6 |
| TIMP1 F | 5′-TCCCTGGAACAGCATGAGTTC-3′ | AF144763 | 7 |
| TIMP1 R | 5′-TGTCGCTCTGCAGTTTGCA-3′ | | 8 |
| ADAMTS4 F | 5′-CCTGGCAACGAGGACTCAAC-3′ | NM_181667.1 | 9 |
| ADAMTS4 R | 51-GGGTAAACAGAATGGCTGTGTCA-3′ | | 10 |
| CD44 F1 | 5′-CGGGTTCATAGAAGGGCATGT-3′ | X62881.1 | 11 |
| CD44 R1 | 5′-TTGTTCGCAGCACAGATGGA-3′ | | 12 |
| Link protein F | 5′-AAGCTGACCTACGACGAAGCG-3′ | NM_174288.1 | 13 |
| Link protein R | 5′-CGCAACGGTCATATCCCAGA-3′ | | 14 |

### Example 5

### Intervertebral Disc Nucleus Pulposus-Derived Stem Cells: A Method of Autologous Cell-Based Therapeutics

### Introduction:

One of the significant challenges facing the development of a regenerative strategy for the treatment of DDD is that significantly degenerative discs become increasingly acellular. It is demonstrated herein that downstream of IL-1β and FasL signaling there is accelerated apoptosis of IVD NP cells. Associated with increasing apoptosis is a progressive loss of the ability of the NP cells to synthesize/repair a viable extracellular matrix. The application of soluble ‘factors’ within the context of a regenerative strategy will be viable only so long as there are sufficient cells/receptor sites available for these factors to act upon. Therefore such ‘factor’ delivery would be reserved for milder forms of degenerative disease such as may occur with either non-surgical patients or at the time of discectomy for acutely herniated IVD.

A cell-based therapy would be required in the case of more advanced degree of degeneration where it is necessary to re-populate the IVD NP. The ideal cell-based biological therapeutic strategy would re-activate homeostatic mechanisms innately inherent within the healthy intervertebral disc (IVD) that are known to decline and ultimately fail with progressive degeneration and replace/re-supply viable cells. The capacity to re-establish equilibrium between catabolic and anabolic tissue remodeling represents the ideal regenerative strategy for the treatment of DDD.

### Methods:

### Colony Formation:

During the course of the hypoxic tissue culture experiments the appearance of what appeared to be ‘sphere’ formation was noted that was suggestive of the classic appearance of stem cell colonies and/or neurospheres. Therefore the tissue culture methods were adjusted and optimized in order to encourage the formation of such colonies and found that colonies could be generated from canine nucleus pulposus cells (notochordal rich discs in particular) within 10 days of culture. Cells were cultured under clonal density (5 cells/μl) in order to avoid miscellaneous ‘clumping’ of cells. We have been able to consistently develop colonies of cells that are consistent with stem cell ‘spheres’ (FIG. 12 A-C).

Briefly, the nucleus pulposus was removed from the IVD from both NCD and CD canines and this has been performed in quadruplicate. 5-6 canine carcasses were obtained immediately after euthanasia and performed the nucleotomy according to our established protocols. The NP tissue was washed twice in Neural Basal A medium (NBA) (recipe below). The tissues were enzymatically digested overnight and the cells recovered the following day 16-18 hour digestion period under hypoxic conditions of 3.5% O₂. The following day the cells were centrifuged and filtered according to our standard practice and the cells counted and placed under clonal density in Neural Basal Medium containing hormone mix, EGF, FGF and for example heparin (see recipe below) within ultra-low adherence tissue culture flasks (CoStar) within hypoxic conditions of 3.5% O₂ (NuAire).

Neurobasal-A medium: (for 500 ml)
Neurobasal-A medium without L-Glutamine 430 ml (Gibco 10888-022, Invitrogen)
5 ml L-glutamine (Gibco 25030-081)
5 ml Penicillin-Streptomycin (Gibco 15140-148)
10 ml B27 (Gibco 17504-044)

Filter through 0.2 uM filter flask.

Neurobasal-A+GFS medium: (100 ml)
90 ml Neurobasal-A
10 ml Hormone mix (filtered)
20 ul EGF (Sigma, 100 μg E-4127)
20 ul FGF (Sigma, 25 μg F-0291)
7.32 ul Heparin (Sigma, 10,000u H-3149)

### Hormone Mix (200 ml)

[Filter through 0.2 uM filter membrane, store @-20 in aliquots.]
75 ml dsf water autoclaved
20 ml 10× DMEM/F12-Gibco
4 ml 30% Glucose (Sigma, G-6132)
3 ml 7.5% NaHCO₃(Sigma, S5761)
1 ml 1M HEPES (sigma, H-3375)
75 ml dsf water

### Add:

*200 mg Transferrin (stored at 4° C.) (Sigma, T-2252)
*50 mg Insulin/18 ml dsf water (stored at 4° C.) (Sigma, I-5500)
*19.25 mg Putrescine/20 ml dsf water (stored at RT) (Sigma, P-7505)
20 μl Selenium (stored at −20° C.) (Sigma, S-9133) (1 mg+1.93 ml dH₂O in bottle)
20 μl Progesterone (stored at −20° C.) (Sigma, P-6149) (Pro.+1.59 ml 95% ETOH)

Together in Falcon Tube

### Note:

add 2 ml of 0.1 N HCl to the Insulin and vortex; once insulin dissolved add it to the 18 ml of dsf water, then add this to solution

### Stock Solutions

### 30% Glucose (Sigma, G-6152)

weight out 30 g of D-Glucose (T-C grade)
add slowly to 100 ml of dsf water
once dissolved, transfer to flow hood and filter sterilize
aliquot into 30 ml/tube and store at 4° C.

### 7.5% NaHCO₃ (Sigma, S-5761)

weight out 7.5 g NaHCO₃ (T-C grade)
add slowly to 100 ml of dsf water
once dissolved, transfer to flow hood and filter sterilize
aliquot into 30 ml/tube and store at 4° C.
   1M HEPES (Sigma. H-3375)
weight out 23.8 g HEPES
add slowly to 80 ml of dsf water
once dissolved, bring up to 100 ml, transfer to flow hood and filter sterilize
aliquot into 30 ml/tube and store at 4° C.

### 10×DMEM (GIBCO 12100-046)/F12 (GIBCO 21700-075)

dissolve one package of DMEM in 200 ml of dsf water
add one package pf F12 nutrient
once dissolved, transfer to flow hood and filter sterilize
store at 4° C.

### FGF2 (Sigma, 25 μg F-0291)

Reconstitute using 500 μl/475 μl Hormone Mix, aliquot 20 μl/tube, store at −20° C.

### EGF (Sigma, 100 μg E-4127)

Reconstitute using 100 μl Hormone Mix, aliquot 20 μl/tube, store at −20° C.

Freeze both of them in 20 μl aliquots

### Heparin (Sigma, 10,000 u H-3149)

Reconstitute 27.32 mg/ml in Hormone Mix
Freeze in 7.32 μl aliquots

### L-glutamine 100×GIBCO 25030

Within 10 days visible colonies appear that are collected by inversion filtering using 40 μm cell strainers that are then re-positioned within a 50 mL Falcon tube and the cell strainer washed with NBA medium. The flow-through is then centrifuged for 3 minutes at 500×g and the cells counted and placed within T75 ultra-low adherence Flasks using 40 mL of NBA supplemented with PSF, EGF, FGF, Hormone mix and heparin. Approximately one week of hypoxic culture (medium replaced each three days) the spheres are trypsinized and using mechanical pipetting the spheres were fragmented to single cells. The single cells are collected using centrifugation within NBA medium containing 1 mg/mL Trypsin inhibitor and finally washed with complete NBA plus additives.

It was sought to determine the numbers of colonies of stem cell ‘spheres’ that were capable of forming from culture. Therefore freshly obtained notochordal cells were seeded at a density of 1500 cells/well onto sterile glass cover slips within 12-well plates and cultured the cells with NBA+EGF+FGF and heparin under hypoxic conditions for three weeks. Thereafter the number of colonies formed were counted and expressed as a fraction of the starting cell number (14 wells were seeded in total).

### Lineage Capacity Evaluation:

Once we had developed the colonies we wished to evaluate their capacity to demonstrate ‘stemness’ by directing their differentiation along specific cell lineages in order to verify their multi-potential. We obtained the colonies and after 1 passage (spheres re-formed) and used trypsin to recover single cells and used various tissue culture methods to induce differentiation along chondrocyte, neural and adipogenic lineages. We used the StemPro chondrogenic and adipogenic kits as per the manufacturer's instructions to induce differentiation along these phenotypic lineages (FIG. 13).

### Chondrogenesis:

Briefly, the cells to be differentiated along chondrogenic lines were seeded on 2.5 cm sterile glass slides within 12-well tissue culture plates in micro-mass culture at a density of 1.6×10⁷ cells/μl in 5 μl droplets and left for 4 hours at 37° C. and 95% humidity. After 4 hours warmed 37° C. chondrogenic medium was added to the cultures and the medium exchanged each 3 days for 4 weeks. After 4 weeks the cultures were stained for aggrecan and collagen type II immunostaining (FIG. 13 A-B).

### Neurogenesis

For neurogenetic differentiation cells obtained from hypoxically generated colonies were seeded at a density of 2×10⁴ cells/cm² using NBA supplemented with 1% FBS and PSF without EGF and FGF supplementation for a minimum of two weeks upon matrigel coated glass slides inserted within 4-well plates (Nunclon-Sigma). The medium was exchanged each 3 days and the cell differentiation followed by microscopic evaluation. At 4 weeks the cell cultures were stained with anti-Beta III tubulin and anti-GFAP antibodies and examined with fluorescent microscopy (FIG. 14 A-B).

### Adipogenesis:

For adipogenic differentiation cells obtained from the same spheres were seeded in MSC growth medium as per the manufacturer's instructions (DMEM low glucose, 10% FBS, GlutaMax and Gentamicin, Invitrogen StemPro Adipogenesis Differentiation Kit) at a density of 1×10⁴ cells/cm² overnight. The next day the media was replaced with StemPro adipocyte differentiation medium according to the manufacturer's instructions with medium exchanges each 3 days for 4 weeks (FIG. 15 A-B).

### In Vivo Neurogenesis:

Studies on the capacity of IVD-derived colony forming stem/progenitor cells to differentiate in vivo along a neural lineage-specifically into myelin-forming cells have been performed. 2.5 μl of canine NCD-derived colony forming stem/progenitor cells were injected at a concentration of 3×10⁶ cells/mL into the brains of newborn shiverer mice-animals with a genetic mutation that renders their central nervous system myelin-deficient. Four weeks post injection the animals were perfused with ice-cold 4% paraformaldehyde, sacrificed and the brains were removed and cryo-preserved using 30% sucrose and held at 4° C. for 24 hours. Then the brains were immersed in OCT within a cryosectioning cassette, mounted and the region of the brain where the injection was performed was serially sectioned at 20 μm, mounted on slides and examined immunohistochemically for the presence of immune-like reactivity to myelin-basic protein using FITC-conjugated anti-human myelin basic protein antibodies (FIG. 16)

### Genomic Characterization:

We were interested in the ability of the IVD NP cells that formed colonies to express classic pluripotential stem cell genes as compared to the total cells obtained from the IVD therefore total RNA was extracted from the IVD NP of the NCD canines as well as from cells obtained from the colonies. Semi-quantitative RT-PCR was performed using cDNA that was reverse transcribed from this RNA and we evaluated for the expression of Oct3/4, Nanog, Sox2, Nestin, BIII, CD133, GFAP, and the housekeeping gene HPRT for the purposes of normalization. The data for these genes is given in FIG. 17. Clearly the pluripotential genes are expressed at very low levels by the total disc cells but highly expressed by the cells obtained from the colony spheres.

### Results:

Stem Cell Colony Formation: Colonies of cells forming spheres that are typical of stem cell colonies from both NCD and CD canine NP IVDs have been consistently generated. Approximately 1% of seeded cells formed colonies based upon the 14 wells containing 1500 cells studied.

Lineage Capacity: We observed robust anti-aggrecan and collagen type II staining and phenotypic appearance classical for chondrocytes after 3 weeks of chondrogenic culture. The appearance of GFAP and Beta III tubulin staining was dramatic as was the phenotypic appearance of the cells that had undergone startling transformation from the original notochordal cell phenotype then colony-appearing cells and finally into neural lineage. GFAP (glial fibrillary associated protein) is a typical marker for glial cells such as astrocytes. Beta III tubulin is commonly used to identify neurons. Finally, the robust oil-red staining was clear evidence that the cells originally from within the NCD canine IVD NP had differentiated into an adipose cell phenotype after 4 weeks of adipogenic culture.

### In Vivo Neurogenesis:

We were able to detect on serially sectioned 20 μm thick sections that there is immune-like reactivity to anti-human myelin basic protein (FIG. 16).

### Pluripotential Gene Expression:

We have further performed qRT-PCR evaluation of total RNA obtained from cells directly from the canine IVD NP as well as from the spheres generated under hypoxic conditions and have verified that the spheres contain cells that express genes unique to pluripotential stem cells: Oct3/4, Sox2, CD133, and the noggin gene. Nanog functions in concert with Oct3/4 and Sox2 to maintain pluripotency. Sox2 is one of the key transcription factors required in induced pluripotent stem cells. CD133 is a well-established marker of stem cell pluripotency. It is barely present within total disc cells but markedly up-regulated within spheres (FIG. 17).

Colony Formation (monolayer): We have generated stem cell colonies within alginate beads and after recovering the colonies from the alginate using sodium citrate methods have plated the colonies upon matrigel-coated glass coverslips (FIG. 18 A-b). These colonies have been examined subsequently using immunohistochemical methods for the presence of certain stem cell markers such as Bill tubulin (FIG. 19A). A typical example of a stem cell colony is given in FIG. 18 (c) (open access web source).

Stem Cell Differentiation Immunohistochemistry: We have plated colony-derived cells upon matrigel-coated coverslips within 4-well plates and stained for the presence of several markers of pluripotential capacity and lineage differentiation. FIG. 19(B) depicts Sox2 expression with peri-nuclear staining of Sox2 immune-like reactivity that co-localizes with DAPI staining of the cell nucleus. Also we have stained these cells for neural cell adhesion molecule (NCAM) immune-like reactivity.

### Summary and Key Implications:

Degenerative disc disease (DDD) is an extremely common and expensive healthcare ailment which to date has no curative strategy. There continues to be no biological “regenerative” strategy for the treatment of DDD, however a therapeutic intervention that may offer restorative qualities to the disc is a much needed and widely sought goal. The ideal biological intervention would re-activate homeostatic mechanisms innately inherent within the healthy intervertebral disc (IVD) that are known to decline and ultimately fail with progressive degeneration. The capacity to re-establish equilibrium between catabolic and anabolic tissue remodeling represents the ideal regenerative strategy for the treatment of DDD.

It has been demonstrated using immunohistochemical methods that cells staining positively for the CD133 surface marker that is expressed by pluripotent stem are present in some human discs obtained at autopsy and surgery [81]. These investigators demonstrated other surface markers characteristic of stem/pluripotential cells and they were able to differentiate these cells into chondrogenic, adipogenic and osteogenic pathways [81]. They did not demonstrate neurogenic differentiation in vitro.

Not only have colonies/spheres herein been generated from hypoxic culture of IVD NP cells but in vivo evidence of the pluripotential differentiation of these cells has been generated and this has been across species. The same methods have been applied to cells obtained from New Zealand White rabbits. Cells were obtained from the lumbar Intervertebral disc Nucleus pulposus using the same methods as described for the dogs. The rabbit cells also form spheres and have the same appearing 3D notochordal cell structures. The results of these experiments provide in vitro and in vivo evidence that notochordal cells contain regenerative qualities that are necessary and sufficient to offer protection of degradation of the NP and secreted soluble factors that provide molecular signals that mitigate cell death and degradation of NP cells induced by IL-1β and IL-1β+FasL. Furthermore, the revolutionary discovery that the NPs of NCD and CD canines contain a robust population of multi-lineage capacity stem/progenitor cells raises the possibility that true regenerative medicine applications could be harnessed with respect to the biological treatment of DDD and other disorders such as neurological disorders involving spinal cord and disorders of the central nervous system. Human discs are formed from notochordal cell precursors just as are all mammals. It is clear that there is a ‘disc niche’ that could be exploited in a revolutionary cell-based therapy in addition to the delivery of the appropriate biologically active molecules that are present within NCCM. Harnessing the regenerative capacity of these cells and the important factors they secrete may lay the cornerstone of biological therapy for the treatment of degenerative disc disease and other disorders such as those of the nervous system.

The ability to deliver factors such as CTGF/CCN2 to the IVD NP that re-establish essential homeostasis and protect against degradation will revolutionize the treatment of DDD. Such a treatment could be performed within the context of a percutaneous delivery of this factor, delivery at the time of surgery such as micro-discectomy in the form of injection and/or the application of a time-delayed delivery of CTGF/CCN2 within the disc or other controlled delivery methods.

With respect to cellular/regenerative strategies, the ability to deliver cells or example, stem cells engineered to differentiate into nucleus pulposus cell phenotype or cells over-expressing CTGF, given that the degenerative disc cell may no longer be able to express CTGF, to the injured/degenerative disc that are capable of assembling a functioning matrix that also secrete self-sustaining factors within a nutrient deficient environment represents a quantum leap in the treatment of DDD. Also, the novel observation that animals deficient in myelin can incorporate progenitor cells obtained across species that can differentiate into cells producing myelin basic protein (oligodendrocyte cells produce myelin) may lead to the possibility of using these cells in patients with spinal injury whereby their own discs may serve as an autologous source of stem cells to be used to treat their own spinal injury. This original research for the very first time offers novel insight into the development of a revolutionary, novel and translational approach to the treatment of degenerative disease of the intervertebral disc and nervous system. Comparison of stem cell-related genes expressed by NCD canine IVD ‘notochordal cells’ with genes expressed by notochordal cell-generated spheres (Notochordal Stem Cell-NCSC). cDNA obtained from NCSC colony spheres provides for robust expression of OCT3/4, nanog Sox2 and elevated expression of CD133 genes by NCSCs. These same genes are much more weakly expressed by cDNA reverse transcribe from RNA obtained from notochordal cells from the total disc nucleus. These changes have yet to be quantified however the above reflect current findings strongly indicative that the colonies/spheres are indeed composed of pluripotential stem cells AND that the NCD IVD NP contains an abundance of cells expressing genes consistent with those of pluripotential stem cells. 1: Oct3/4, 2: Nanog, 3: Sox2, 4: Nestin, 5: Beta III, 6: CD133, 7: GFAP, 8: HPRT, 9: size standard.

### REFERENCES FOR EXAMPLES 3, 4 AND 5

1. Goetzel R Z, et al., The health and productivity cost burden of the “top 10” physical and mental conditions affecting six large U.S. employers in 1999. Journal of Occupational and Environmental Medicine, 2003. 45(1): p. 5-14.
2. Barrionuevo F, et al., Sox9 is required for notochord maintenance in mice. Developmental Biology, 2006. 295: p. 128-140.
3. Aguiar D J, Johnson S L, and Oegema Jr T R, Notochordal cells interact with nucleus pulposus cells: regulation of proteoglycan synthesis. Experimental Cell Research 1999. 246: p. 129-137.
4. Erwin W M and Inman R D, Notochord cells regulate intervertebral disc chondrocyte proteoglycan production and cell proliferation. Spine, 2006. May 1(31(10)): p. 1094-9.
5. Erwin W M, et al., Nucleus pulposus notochord cells secrete connective tissue growth factor and up-regulate proteoglycan expression by intervertebral disc chondrocytes. Arthritis and Rheumatism, 2006. December 54(12): p. 3859-67.
6. Arnott J A, et al., Connective tissue growth factor (CTGF/CCN2) is a downstream mediator for TGF-beta 1-induced extracellular matrix production in osteoblasts. Journal of Cell Physiology, 2007. March; 210(3): p. 843-52.
7. Behrens A, et al., Impaired intervertebral disc formation in the absence of Jun. Development 2003. January; 130(1): p. 103-9
8. Holm S M A, Urban J, and et al, Nutrition of the intervertebral disc. Solute transport and metabolism. Connective Tissue Research 1981.8: p. 101-119.
9. Urban J P, The role of the physicochemical environment in determining disc cell behaviour. Biochem Society Transactions 2002 November; 30 (Pt 6): p. 858-64.
10. Urban J P, Smith S, and Fairbank J C, Nutrition of the intervertebral disc. Spine 2004 Dec. 1; 29(23): p. 2700-9.
11. Selard E, Shirazi-Adl A, and Urban J P, Finite element study of nutrient diffusion in the human intervertebral disc. Spine 2003. Sep. 1; 28 (17): p. 1945-53.
12. Kuroki K C, et al., The effects of TIMP-1 and -2 on canine chondrocytes cultured in three-dimensional agarose culture system. Osteoarthritis and Cartilage 2003. 11: p. 625-635.
13. Vadala G, et al., Coculture of bone marrow mesenchymal stem cells and nucleus pulposus cells modulate gene expression profile without cell fusion. Spine, 2008. 33(8): p. 870-876.
14. Zhang Y, et al., Comparative effects of bone morphogenetic proteins and sox9 overexpression on matrix accumulation by bovine anulus fibrosus cells. Spine, 2007. 32(23): p. 2515-2520.
15. Thompson J P, Oegema Jr. T R, and Bradford D S, Stimulation of mature canine intervertebral disc by growth factors. Spine, 1991. 16(3): p. 253-260.
16. Takegami Kenji, et al., Osteogenic Protein-1 Enhances Matrix Replenishment by Intervertebral Disc Cells Previously Exposed to Interleukin-1. Spine 2002. 27(12 June 15): p. 1318-1325.
17. Tim Yoon S, et al., The effect of bone morphogenetic protein-2 on rat intervertebral disc cells in vitro. Spine, 2003. 28(16): p. 1773-80.
18. Yoon S T, et al., LMP-1 upregulates intervertebral disc cell production of proteoglycans and BMPs in vitro and in vivo. Spine, 2004. 29(23): p. 2603-2611.
19. Paul R, et al., Potential use of Sox9 gene therapy for intervertebral degenerative disc disease. Spine, 2003. 28(8): p. 755-763.
20. Spangenberg K M, P. G., Trahan C A, Randolph M A, Bonassar L J., Histomorphic analysis of a cell-based model of cartilage repair. Tissue engineering in orthopaedic surgery, 2002. 8(5): p. 839-846.
21. Keller R, et al., Mechanisms of convergence and extension by cell intercalation. Philosophical Transactions of the Royal Society-Biological Sciences, 2000. 355: p. 897-922.
22. Pourquie, O., Vertebrate Somitogenesis. Annual Reviews-Cell and Developmental Biology, 2001. 17: p. 311-50.
23. Ghosh P, The biology of the intervertebral disc 1. 1988, Boca Raton Fla.: CRC Press.
24. Hirano S H R and Najita M, Morphological analysis of the role of the neural tube and notochord in the development of somites. Anat Embryol 1995; 192: p. 445-457.
25. Shih J and Keller R, Cell motility driving mediolateral intercalation in explants of Xenopus laevis. Development, 1992. 116: p. 915-930.
26. Rajpurohit R, et al., Phenotypic characteristics of the nucleus pulposus: expression of hypoxia inducing factor-1, glucose transporter-1 and MMP-2. Cell and Tissue Research, 2002. June; 308(3): p. 401-7. Epub 2002 May 25.
27. Agrawal A, et al., Normoxic stabilization of HIF-1alpha drives glycolytic metabolism and regulates aggrecan gene expression in nucleus pulposus cells of the rat intervertebral disk. American Journal of Physiology-Cell Physiology 2007. August; 293 (2): p. C621-31.
28. Higgins D F, et al., Hypoxic induction of CTGF is directly mediated by Hif-1. American Journal of Physiology-Renal Physiology, 2004. 297(August 17): p. F1223-F1232.
29. Schipani E, et al., HIF-1a is essential for chondrocyte growth arrest and survival. Genes Development, 2001. 15: p. 2865-2876.
30. Hishikawa K, Nakaki T, and Tomoko F, Connective Tissue Growth Factor induces apoptosis via caspace-3 cultured human aortic smooth muscle cells. European Spine Journal, 2000. 392(19-22).
31. Brigstock D R, The CCN family: a new stimulus package. Journal of Endocrinology, 2003. 178: p. 169-175.
32. Ivkovic S, et al., Connective tissue growth factor coordinates chondrogenesis and angiogenesis during skeletal development. Development and Disease, 2003. 130: p. 2779-2791.
33. Risbud M V, et al., Nucleus pulposus cells upregulate PI3K/Akt and MEK/ERK signaling pathways under hypoxic conditions and resist apoptosis induced by serum withdrawl. Spine, 2005. 30(8): p. 882-889.
34. Cappello R, et al., Notochordal cell produce and assemble extracellular matrix in a distinct manner, which may be responsible for the maintenance of a healthy nucleus pulposus. Spine, 2006. April 15-31(8): p. 873-882.
35. Oegema Jr. TR, The role of disc cell heterogeneity in determining disc biochemistry: a speculation Biochemical Society Transactions 2002. 30(6): p. 839-844.
36. Aguiar D J, Johnson S L, and Oegema T R, Notochordal cells interact with nucleus pulposus cells: regulation of proteoglycan synthesis. Experimental Cell Research, 1999. 246: p. 129-137.
37. Oegema Jr. TR, The role of disc cell heterogeneity in determining disc biochemistry: a speculation. Biochemical Society Transactions, 2002. 30(6): p. 839-844.
38. Erwin W M and Inman R D, Notochord cells regulate intervertebral disc chondrocyte proteoglycan production and cell proliferation. Spine, 2006. 31(10): p. 1094-1099.
39. Erwin W M, et al., Nucleus pulposus notochord cells secrete connective tissue growth factor and upregulate proteoglycan expression by intervertebral disc chondrocytes. Arthritis and Rheumatism, 2006. 54(12): p. 3859-3867.
40. Erwin W M, et al., The Regenerative Capacity of the Notochordal Cell: Tissue Constructs Generated in Vitro under Hypoxic Conditions. The Journal of Neurosurgery-Spine, 2009. 10: p. 513-522.
41. Studer Rebecca K, et al., p38MAPK inhibition modulates rabbit nucleus pulposus cell response to II-1. Journal of Orthopaedic Research, 2008. 26(7): p. 991-998.
42. Ulrich Jill A, et al., Repeated disc injury causes persistent inflammation. Spine, 2007. 32(5): p. 2812-2819.
43. Hornebeck W, Down-regulation of tissue inhibitor of matrix metalloproteinase-1 (TIMP-1) in aged skin contributes to matrix degradation and impaired cell growth and survival. Pathologies Biologie, 2003. 51: p. 569-573.
44. Le Maitre C, et al., Matrix synthesis and degradation in human intervertebral disc degeneration. Biochemical Society Transactions, 2007. 35(4): p. 652-655.
45. Le Maitre C L, Freemont A J, and Hoyland J A, The role of interleukin-1 in the pathogenesis of human intervertebral disc degeneration. Arthritis Research and Therapy, 2005. 7(4; April 1:R732-45. Epub).
46. Cui L-Y, et al., IL-1 beta sensitizes rat intervertebral disc cells to Fas ligand mediated apoptosis in vitro. Acta Pharmacologica Sinica, 2007. 28: p. 1671-1676.
47. Fitzgerald J B, Jin M, and Grodzinsky A J, Shear and compression differentially regulate clusters of functionally-related temporal transcription patterns in cartilage tissue-supplemental material. Journal of Biolological Chemistry, 2006. 281(34): p. 24095-24103.
48. Nomura S, et al., Alterations in gastric mucosal lineages induced by acute oxyntic atrophy in wild-type and gastrin-deficient mice. American Journal of Physiology Gastrointestinal Liver Physiology, 2005. 288: p. 362-375.
49. Le Maitre C L, Hoyland J A, and Freemont A J, Catabolic cytokine expression in degenerate and herniated human intervertebral discs: IL-1beta and TNFalpha expression profile. Arthritis Research and Therapy, 2007. 9(4 (Epub)): p. R77.
50. Pocked A J, et al., Modified expression of the ADAMTS enzymes and tissue inhibitort of metalloproteinases 3 during human intervertebral disc degeneration. Arthritis and Rheumatism, 2009. 60(2): p. 482-491.
51. Lotz J C, H. A., Walsh A L, Palmer E I, and Chin J R, Mechanobiology of the intervertebral disc. Biochemical Society Transactions, 2002. 30(6): p. 853-858.
52. Urban J P, The role of the physicochemical environment in determining disc cell behaviour. Biochemical Society Transactions, 2002. November; 30 (Pt 6): p. 858-864.
53. Ahn Howard S and Masuda K, Relevance of in vitro and in vivo models for intervertebrfal disc degeneration. Journal of Bone and Joint Surgery, 2006. 88-A(Supplement 2): p. 88-94.
54. Antoniou John, et al., The Human Lumbar Intervertebral disc-evidence for changes in the biosynthesis and denaturaion of the extracellular matrix and growth, maturation, ageing, and degeneration. Journal of Clinical Investigation, 1996. 98(4): p. 996-1003.
55. Nomura T, et al., Nucleus pulposus allograft retards intervertebral disc degeneration. Clinical Orthopaedics and Related Research, 2001. 389: p. 94-101.
56. Goupille P, et al., Matrix metalloproteinases: the clue to intervertebral disc degeneration? Spine, 1998. 23(14): p. 1612-1626.
57. Kim K-W, et al., An autocrine or paracrine Fas-mediated counterattack: A potential mechanism for apoptosis of notochord cells in intact rat nucleus. Spine, 2005. 31(11): p. 1247-1251.
58. Park Jong-Beom, et al., Expression of Fas receptor on disc cells in herniated lumbar disc tissue. Spine, 2001. January 15; 26(23): p. 142-146.
59. Park J B, et al., Anti-apoptotic effects of caspase inhibitors on rat intervertebral discs. Journal of Bone and Joint Surgery Am, 2006. April88(4): p. 771-779.
60. Mwale F, Roughley P, and Anmtoniou J, Distinction between the extracellular matrix of the nucleus pulposus and hyaline cartilage: A requisite for tissue engineering of intervertebral disc. European Cells and Materials, 2004. 8: p. 58-64.
61. Ghosh P, The biology of the intervertebral disc. Vol. 1. 1988, Boca Raton, FI: CRC Press.
62. Hayes A J, Benjamin M, and Raphs J R, Extracellular matrix in development of the intervertebral disc. Matrix Biology, 2001. 20: p. 107-121.
63. Poiraudeau S, et al., Phenotypic characteristics of rabbit intervertebral disc cells. Spine, 1999. 24(9): p. 837-844.
64. Gardner Jessica and Ghorpade Anuja, Tissue inhibitor of metalloproteinase (TIMP-1)-The TIMPed balance of matrix metalloproteinases in the central nervous system. Journal of Neuroscience Research, 2003. 74(6): p. 801-806.
65. Bonassar L J, et al., Inhibition of cartilage degradation and changes in physical properties induced by IL-18 and retinoic acie using matrix metalloproteinase inhibitors. Arch Biochem Biophys, 1997. 344: p. 404-412.
66. Ghosh P, Matrix metalloproteinases: The clue to intervertebral disc degeneration? Spine, 1998. 23(14): p. 1626.
67. Liu J, Roughley P J, and Mort J S, Identification of human intervertebral disc stromelysin and its involvement in matrix degradation. J Orthop Res, 1991. 9: p. 568-575.
68. Matsui Y, et al., The involvement of matrix metalloproteinases and inflammation in lumbar disc herniation. Spine, 1998. April 15; 23(8): p. 863-868.
69. Kuroki K, et al., The effects of TIMP-1 and -2 on canine chondrocytes cultured in three-dimensional agarose culture system. Osteoarthritis Cartilage, 2003. 11(9): p. 625-635.
70. Ellis A J, et al., The prevention of collagen breakdown in bovine nasal cartilage by TIMP, TIMP-2 and a low molecular weight synthetic inhibitor. Biochemical and Biophysical Research Communications, 1994. 210: p. 94-101.
71. Gomez D E, et al., Tissue inhibitors of metalloproteinases-structure regulation and biological functions. European Journal of Cell Biology, 1997. 74(2): p. 111-122.
72. Tan Hiang Khoon, et al., Tissue inhibitor of metalloproteinase 1 inhibits excitotoxic cell death in neurons. Molecular and Cellular Neuroscience, 2003. 22: p. 98-106.
73. Davidson M L, et al., TIMP-1 gene deficiency increases tumour cell sensitivity to chemotherapy-induced apoptosis. British Journal of Cancer, 2006. 95: p. 1114-1120.
74. Braund K G, Morphological studies of the canine intervertebral disc. The assignment of the beagle to the achondroplastic classification. Research in Veterinary Science, 1975. 9: p. 167-172.
75. Cole T K, et al., The proteoglycans of the canine intervertebral disc. Biochimica et Biophysica Acta, 1985(839): p. 127-138.
76. Julovi Sohel M, et al., Inhibition of interleukin-1B stimulated production of matrix metalloproteinases by hyaluronan via CD44 in human articular cartilage. Arthritis and Rheumatism, 2004. 50(2): p. 516-525.
77. Ghosh P, The biology of the intervertebral disc. Vol. 2. 1988, Boca Raton, FI: CRC Press.
78. Thompson J P, Oegema Jr. TR, and Bradford D S, Stimulation of mature canine intervertebral disc by growth factors. Spine, 1991. 16(3): p. 253-260.
79. Hohaus C, et al., Cell transplantation in lumbar spine disc degeneration disease. European Spine Journal, 2008. December 17(Suppl 4): p. 592-503.
80. Matsuzaki H, et al., Allografting intervertebral discs in dogs: a possible clinical application. Spine, 1996. 15(21): p. 178-183.
81. Risbud M V, et al., Evidence for skeletal progenitor cells in the degenerate human intervertebral disc. Spine, 2007. 32(23): p. 2537-2544.
82. Femor B, et al., European Cell and Materials, 2007. Vol. 13: p. 56-65.
83. Alexanian A R., et al. Experimental Neurology, 2005. 191: p. 193-197.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of maintaining and/or expanding an in vitro population of hypoxia compartment cells comprising culturing said population of cells under suitable cell culture conditions, wherein the population of hypoxia compartment cells is exposed to an oxygen concentration of between about 1.5% and about 10%, preferably between about 2% and about 5%, optionally in an oxygen controlled environment.

2. The method of claim 1, wherein the hypoxia compartment cells comprises intervertebral disc (IVD) cells selected from one or more of nucleus pulposus (NP) cells, such as chondrocyte-like cells, progenitor cells and/or notochordal cells, and annulus fibrous cells including primary notochordal cells.

3. (canceled)

4. (canceled)

5. The method of claim 1, wherein the hypoxia compartment cells form cell aggregates, wherein the oxygen concentration selected allows the population of cells to produce cell aggregates comprising one or more of extracellular matrix, proteoglycan, and/or fibrils, wherein the cell aggregates are optionally oval or crescent shaped, optionally with viscoelastic properties.

6. The method of claim 1, wherein the population of hypoxia compartment cells is comprised in a biocompatible gel such as a hydrogel, wherein the biocompatible gel comprises an anionic polysaccharide, preferably alginate such as sodium alginate, and a divalent cation salt such as CaCl2.

7. (canceled)

8. (canceled)

9. (canceled)

10. (canceled)

11. The method of claim 1, wherein the population of cells expanded under hypoxic conditions is characterized as having on average at least a 1.5, 1.8, 2.1, 2.4, 2.7 or 3 fold increase in cell area compared to a population of cells exposed to an oxygen concentration of about 21%.

12. (canceled)

13. (canceled)

14. A method of generating and/or enriching for progenitor cells comprising: providing a population of cells comprising and/or consisting of hypoxia compartment cells; culturing the population of cells under suitable conditions, wherein the population cells is exposed to an oxygen concentration of between about 1.5% and about 10%, preferably between about 2% and about 5%, optionally in an oxygen controlled environment for a sufficient time for the population of hypoxia compartment progenitor cells to form spherelike conformations; and isolating the spherelike conformations comprising progenitor cells.

15. The method of claim 14 wherein the population of cells comprising and/or consisting of hypoxia compartment cells is obtained from a hypoxia compartment tissue, wherein the hypoxia compartment tissue is an intervertebral disc, preferably nucleus pulposus tissue and wherein the method of isolating the population of cells comprising isolating nucleus pulposus (NP) tissue, contacting the NP tissue with one or more enzymes, and optionally filtering said cells to obtain the population of cells.

16. (canceled)

17. The method of claim 15, wherein isolating the cell aggregates comprises for methods wherein the cells are suspended in a biocompatible gel, dissolving the gel, for example with sodium citrate, separating the cells for example by density gradient separation, and recovering layers comprising the cell aggregates, for example that correspond to the 1.014 and 1.021 specific gravity levels of a Percoll density gradient developed in 1 mL volumes for example, at 1.007, 1.014, 1.021, 1.028 and 1.035 specific gravity steps, centrifuged at 200×G for 20 minutes; and culturing the recovered layers, for example by resuspending the recovered layers in a biocompatible gel and culturing for a suitable period, for example about 3 weeks, about 4 weeks, about 5 weeks or about 6 weeks.

18. The method of claim 14, wherein the conditions comprise culturing the cells in a suspension culture, optionally in a basal medium without serum, for example Neuralbasal A™, optionally comprising hormones, and/or growth factors.

19. (canceled)

20. The method of claim 15 wherein the population of cells comprises notochordal cells, preferably primary notochordal cells.

21. The method of claim 14, wherein the hypoxia compartment cells comprise NP cells and the conditions are suitable for producing neuronal progenitor cells.

22. (canceled)

23. (canceled)

24. The method of claim 20, wherein the spherelike conformations comprise at least 0.2%, at least 0.3%, at least 0.4%, at least 0.5%, at least 0.6%, at least 0.7% at least 0.8% at least 0.9% or at least 1.0% progenitor cells, for example neuronal progenitor cells.

25. A method for testing sensitivity of a population of hypoxia compartment cells to a test agent or combination, comprising maintaining and/or expanding an in vitro population of hypoxia compartment cells according to the method of claim 1; adding the test agent or combination and determining sensitivity of said cells to test agent or combination.

26. (canceled)

27. A method of producing a therapeutic hypoxia compartment cell comprising isolating a hypoxia compartment cell and maintaining and/or expanding said cells according to the method of claim 1, and optionally isolating the cell.

28. An isolated cell, isolated or derived from a hypoxia compartment tissue, maintained and/or expanded according to the method of claim 1, optionally, wherein the hypoxia compartment cell is a NP cell.

29. (canceled)

30. (canceled)

31. A method of producing nucleus pulposa conditioned medium (NPCM) or notochordal cell conditioned medium (NCCM) comprising culturing a population of NP cells and/or notochordal cells, preferably primary notochordal cells, in a cell culture medium under suitable cell culture conditions, wherein the population of NP cells and/or notochordal cells is exposed to an oxygen concentration of between about 1.5% and about 10%, preferably between about 2% and about 5%, and cultured preferably in basal culture medium devoid of serum and/or growth factors, for a suitable culture period, for example of at least 12 hours, at least 24 hours, at least 36 hours, at least 48 hours, at least 60 hours or at least 72 hours, optionally in an oxygen controlled environment, thereby producing NPCM and/or NCCM.

32. (canceled)

33. (canceled)

34. (canceled)

35. (canceled)

36. The method of claim 31, wherein the NPCM or the NCCM is fractionated, for example to isolate a fraction containing factors less than about 80 kDa, less than about 70 kDa, less than about 60 kDa, less than about 50 kDa, or less than about 40 kDa.

37. A method of inhibiting nucleus pulposus cell death comprising contacting a nucleus pulposus cell that is being exposed or will be exposed to a cell death signal with the NP or NC conditioned media according to claim 31 and/or contacting the nucleus pulposus cell with CTGF, preferably recombinant CTGF, more preferably recombinant human CTGF (rhCTGF) and/or an isolated cell or population according to claim 28, and/or a cell that expresses CTGF.

38. (canceled)

39. A method of inhibiting inflammatory cytokine and death receptor induced gene expression modulation in a NP cell comprising contacting the NP cell with the nucleus pulposus cell conditioned medium (NPCM), the notochordal cell conditioned medium (NCCM), CTGF or an isolated cell or population of claim 28, wherein the matrix gene is selected from aggrecan link, CD44 receptor, collagen type II, MMP3, TIMP-1, wherein contact between the NP cell and the NCCM, CTGF or isolated cell inhibits matrix gene expression modulation induced by inflammatory cytokines.

40. A method of inhibiting nucleus pulposus degeneration in a subject comprising administering, or contacting a population of the subject's NP cells, with a nucleus pulposus cell conditioned medium (NPCM) or a notochordal cell conditioned medium (NCCM), CTGF or an isolated cell or population of claim 28.

41. (canceled)

42. (canceled)

43. A method of treating degenerative disc disease (DDD) in a subject comprising administering NPCM, NCCM, CTGF or an isolated cell or population of claim 28, to a subject in need thereof.

44. (canceled)

45. The method of claim 43, wherein the treatment inhibits ECM degradation.

46. (canceled)

47. A method of treating a neuronal disease characterized by loss of cells, for example spinal cord injury, comprising administering NPCM, NCCM, CTGF or an isolated cell or population of claim 28, to a subject in need thereof.

48. The method of claim 37, wherein the concentration of CTGF administered is from about 0.1 ng/mL to about 1 ng/mL, from about 1.1 ng/mL to about 10 ng/mL, from about 11 ng/mL to about 50 ng/mL, from about 51 mg/mL to about 100 ng/mL.

49. The method of claim 37, wherein the population of cells is autologous.

50. (canceled)

51. (canceled)

52. A method of differentiating a cell comprising obtaining a spherelike conformation for example according to the methods of claim 1, culturing the spherelike conformation in a differentiation culture medium under suitable conditions to obtain a differentiated cell.

53. (canceled)

54. (canceled)
